(19) United States
(12) Patent Application Publication
(10) Pub. No.: US 2017/0283885 A1
(43) Pub. Date: Oct. 5, 2017

(54) ALGORITHMS FOR GENE SIGNATURE-BASED PREDICTOR OF SENSITIVITY TO MDM2 INHIBITORS

(71) Applicant: DAIICHI SANKYO COMPANY, LIMITED, Tokyo (JP)

(72) Inventors: Ngai-chiu Archie TSE, Long Island City, NY (US); Kenji NAKAMARU, Tokyo (JP); Koichi TAZAKI, Tokyo (JP); Kenji WATANABE, Tokyo (JP); Takahiko SEKI, Tokyo (JP)

(73) Assignee: DAIICHI SANKYO COMPANY, LIMITED, Tokyo (JP)

(21) Appl. No.: 15/510,892

(22) PCT Filed: Oct. 9, 2015
(86) PCT No.: PCT/JP2015/079389

§ 371 Date: Mar. 13, 2017

Related U.S. Application Data
(60) Provisional application No. 62/061,992, filed on Oct. 9, 2014.

(51) Int. Cl. C12Q 1/68; G01N 33/574; A61K 31/499

(52) U.S. Cl. CPC C12Q 1/6886; A61K 31/499; G01N 33/574; C12Q 2600/106; C12Q 2600/158

(57) ABSTRACT

Provided are gene signatures that are predictive of the sensitivity of a cancer or tumor to an MDM2i or an antagonist of the MDM2-p53 interaction. Differentially expressed genes in the provided gene signatures serve as biomarkers for determining and assessing the sensitivity of cancer and tumor samples to treatment or therapy with an MDM2i. Also provided are methods of determining MDM2i sensitivity of a test sample such as different cancer and tumor types and subtypes, based on the expression of genes in the MDM2i sensitive gene signatures in reference samples and the test sample even if all of the MDM2i sensitivities of the reference samples are unknown, and treating individuals with an MDM2i if their cancers are determined to be MDM2i-sensitive, based on the practice of the described methods. TP53 gene and p53 protein status can also be determined for the samples undergoing analysis for MDM2i sensitivity.

### FIELD OF INVENTION

The present invention relates generally to gene signatures and gene expression profiles which provide predictive molecular tools for clinical application. The invention also relates to methods of predicting the sensitivity of cancers or tumors to anticancer drugs that can influence the treatment of the cancers or tumors, particularly inhibitors of MDM2 activity and antagonists of the interaction of MDM2 and p53 proteins. The invention further relates to the use of such gene signatures as cancer biomarkers and companion diagnostics for assisting medical practitioners and patients with more effective and individualized cancer and tumor treatments.

### BACKGROUND OF INVENTION

The treatment of cancers is evolving from the use of non-specific cytotoxic agents that affect both cancer and normal cells to more individualized and targeted cancer therapies. Targeted therapies can involve the determination of unique genetic signatures of cancer cells to yield more directed treatments with less toxicity to and greater efficacy for those individuals undergoing cancer treatment and therapy.

To date, treatments for cancer patients routinely rely on agents and regimens that have demonstrated efficacy in randomized clinical trials that typically involve hundreds of subjects. Such treatments are neither individualized nor targeted to an individual patient's cancer or disease and may frequently result in ineffective cancer treatment. Such unsuccessful or subpar treatment for cancer patients may result in unnecessary toxicity, disease progression, and mortality for the patient, and ultimately, higher costs of health care.

The development and progression of certain tumors and cancers can involve an interplay between cellular molecules that ultimately affect cell growth arrest and death. Two molecules that have been determined to play a significant role in cancer are the p53 protein and the Mouse Double Minute 2 (MDM2) protein, also known as Human Double Minute 2 (HDM2).

The p53 tumor suppressor protein (encoded by the TP53 gene) is a key transcriptional regulator that responds to a variety of cellular stresses, e.g., DNA damage, UV irradiation and hypoxia. The p53 protein regulates vital cellular processes such as DNA repair, cell-cycle progression, angiogenesis and apoptosis; its activation can initiate a variety of molecules and downstream pathways in affected cells. These p53-dependent pathways shut down damaged cells through either cell-cycle arrest or apoptosis. Loss or inhibition of p53 function and activity is believed to be a contributing factor in many cases of cancer.

MDM2 is a negative regulator of the p53 tumor suppressor protein. The 90 kDa MDM2 protein contains a p53 binding domain at its N-terminus and a RING (really interesting gene) domain at its C-terminus, which functions as an E3 ligase that ubiquinates p53. The activation of wild-type p53 by cell stimuli and stresses results in the binding of MDM2 to p53 at the N-terminus to inhibit the transcriptional activation of p53 and promote the degradation of p53 via the ubiquitin-proteosome pathway. Thus, MDM2 can interfere with p53-mediated apoptosis and arrest of cancer cell proliferation, attributing a significant oncogenic activity to MDM2 in cancer cells. In some cases, MDM2 can cause carcinogenesis independent of the p53 pathway, for example, in cells which possess an alternative splice form of MDM2. (H. A. Steinman et al., 2004, J. Biol. Chem., 279(6):4877-4886). In addition, about 50% of human cancers are observed to have a mutation in or deletion of the TP53 gene. MDM2 is overexpressed in a number of human cancers, including, for example, melanoma, non-small cell lung cancer (NSCLC), breast cancer, esophageal cancer, leukemia, non-Hodgkin's lymphoma and sarcoma. Overexpression of MDM2 has been reported to correlate positively with poor prognosis in individuals having sarcoma, glioma and acute lymphoblastic leukemia (ALL).

The ability to identify and determine which individuals undergoing treatment for cancer will or will not respond to a given treatment, drug, compound, or therapy is the cornerstone for a more personalized and directed approach to successful current and future cancer treatments. On the basis of diagnostic systems involving gene expression profiles or gene signatures as they relate to and identify the sensitivity or resistance of cancer and tumor cells to given anticancer drugs and agents, the medical practitioner and clinician will be better able to tailor a cancer treatment by determining whether the gene signature of a patient's cancer or tumor cells and tissue samples is one that is indicative of sensitivity or resistance to an anticancer drug, agent, or chemotherapeutic.

In order to provide safer, more efficient, directed and economical cancer treatments, cost-effective tools and systems for predicting and assessing which cancers, and the individuals afflicted with such cancers, will be sensitive or resistant to a given treatment or drug, are profoundly needed. Such tools, e.g., a companion diagnostic involving a gene signature related to drug sensitivity, would be beneficial to clinicians and cancer patients for use at various stages of patient disease and the treatment thereof, for example, to determine whether a drug treatment should be initiated, to predict the efficacy of a drug treatment, or to assess post-treatment status of an individual afflicted with cancer, if indicated or desired, and generally to provide better guidance for patient treatment decisions.

### SUMMARY OF INVENTION

Provided herein are methods, systems, platforms, reagents and kits involving gene signatures and gene expression profiles that are indicative of the sensitivity of a cancer or tumor to a chemotherapeutic or anti-cancer agent, drug, compound, or a combination thereof. More specifically, the gene signatures and gene expression profiles of the invention can be used to predict clinical outcome, such as treatment response or survival, of patients with cancers and tumors who are treated with an agent that inhibits the activity of the MDM2 protein. As used herein, the term “MDM2 inhibitor” is designated and is synonymous with “MDM2i”.

The gene signatures of the invention and the methods of detecting the expression of genes within the gene signatures allow the identification and determination of those individuals afflicted with cancer, tumors, or neoplasms who may, or who are likely to, respond to an MDM2i drug or drug combination.

The gene signatures of the invention and the methods of detecting differentially expressed genes in the gene signatures afford a convenient and efficient means of predicting the sensitivity of cancers and tumors to treatment with an MDM2i. The gene signatures and methods of the invention also are useful in predicting the likelihood of effectively treating a patient having a cancer or tumor with a therapy or regimen involving an MDM2i, thereby providing information and guidance for a more directed and personalized cancer treatment. The invention further provides methods and systems that can yield cost-effective and accurate results regarding a cancer's or tumor's sensitivity to a treatment involving an MDM2i, or a candidate MDM2i, to improve customized and individualized cancer therapy regimens using MDM2 inhibitors. MDM2i treatable cancers and tumors include, but are not limited to, leukemias, lymphomas, myelomas, melanomas, sarcomas and carcinomas.

In an aspect, the invention provides a gene signature, also called a gene expression signature or an MDM2i gene sensitivity signature herein, that is associated with a cellular response to the inhibition of MDM2 in a cancer or tumor sample, including cancer or tumor tissue, cells derived therefrom, and the like. While not wishing to be bound by theory, it will be understood that inhibiting the activity of MDM2 can, in many cases, be considered synonymous with antagonizing the interaction of the MDM2 protein with the p53 protein within a cell.

More particularly, an MDM2i gene sensitivity signature of the invention provides a profile of the genes, or a subset of genes, whose differential expression in a cancer or tumor sample, or cells derived therefrom, relative to a control, predicts or indicates the sensitivity of the cancer or tumor sample to an MDM2i drug or compound. A cancer or tumor sample that is sensitive to an MDM2i will, in some embodiments, have increased expression of at least three or at least four genes within the MDM2i gene sensitivity signatures described herein and will optimally exhibit a cytotoxic response to the inhibitor, for example, as indicated by cell death, senescence, apoptosis, decrease or cessation of cell mobility and/or growth, and the like.

According to an aspect of the invention, the differential expression in cancer or tumor samples or cells of at least three genes, at least four genes, or all of the genes contained in the gene signature of FIGS. 1A-1E is predictive of sensitivity of the samples or cells to an MDM2i. In another aspect, the differential expression in cancer or tumor samples or cells of at least three, at least four, or all, of the genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC is predictive of sensitivity of the samples or cells to an MDM2i. These forty genes are contained in an MDM2i sensitivity gene signature of the invention and constitute a subset of the genes listed in FIGS. 1A-1E. In another aspect, the differential expression in cancer or tumor samples or cells of at least three, at least four, or all, of the genes MDM2, CDKN1A, ZMAT3, DDB2, FDXR, RPS27L, BAX, RRM2B, SESN1, CCNG1, XPC, TNFRSF10B and AEN. In another aspect, the differential expression in cancer or tumor samples or cells of at least three, or all, of the genes RPS27L, FDXR, CDKN1A and AEN (and optionally MDM2) is predictive of sensitivity of the samples or cells to an MDM2i. In specific embodiments, the differential expression in cancer or tumor samples or cells of at least 3, 4, 5, 10, 15, 20, 25, 30, 35, 40, 45, 50 or more genes contained in the gene signature of FIGS. 1A-1E is predictive of sensitivity of the samples or cells to an MDM2i. In specific embodiments, differential expression is increased expression levels of mRNA or protein detected or identified in a cancer or tumor sample or cell.

In an aspect, the invention provides a method of predicting the sensitivity of a subject's cancer or tumor to MDM2 inhibitor treatment, in which the method involves measuring the levels of expression of at least three or at least four genes selected from the genes listed in FIGS. 1A-1E in a cancer or tumor sample obtained from the subject.

In an aspect, the invention provides a method of treating a subject having a cancer or tumor, comprising a) assessing the sensitivity of the subject's cancer or tumor to MDM2 inhibitor treatment, which involves measuring the levels of expression of at least three or at least four genes selected from the genes listed in FIGS. 1A-1E in a cancer or tumor sample obtained from the subject; and b) administering to the subject an effective amount of an MDM2 inhibitor to treat the cancer or tumor, if the assessment indicates that the cancer or tumor is sensitive to the MDM2 inhibitor.

In general, treatment with an MDM2i results in p53 tumor suppressor function or activity in cancer or tumor cells, particularly in those possessing a functional p53, including wild-type or non-mutated p53, leading to effective anti-tumor effects, such as apoptosis, growth inhibition, senescence, or tumor cell death. Such anti-tumor effects typically involve the activation of p53 downstream pathways, which include, but are not limited to, caspase activation or inhibition of cyclin-dependent kinases.

Thus, in an aspect, the methods of the invention involve detecting or measuring in a patient's cancer or tumor sample the differential expression of genes contained in a gene signature of the invention relative to a control as indicative of MDM2i sensitivity of the cancer or tumor, and can further involve an assessment of the functional status of the TP53 gene and/or the p53 protein in the cancer or tumor sample, which is undergoing MDM2i sensitivity analysis or evaluation. As used herein, “p53” refers to the suppressor protein and “TP53” refers to the gene that encodes the p53 suppressor protein. A functional p53 protein has retained its ability to transcriptionally activate the expression of downstream molecules, leading to tumor growth suppression and/or apoptosis. In an aspect, an active or functional p53 protein may result from a wild type TP53 gene status; or a mutated TP53 gene status that does not adversely affect p53 protein activity or function; or the absence of a p53 inhibitor or inhibitory agent, such as, for example, the Human Papilloma Virus E6 oncoprotein (HPV E6). In another aspect, TP53 is wild type and active, and the p53 protein is active and functional. In another aspect, the expression of MDM2i sensitive gene signature genes, as well as TP53 gene status and/or p53 protein status, are determined in cancer or tumor samples undergoing evaluation for MDM2i sensitivity.

In an aspect, the invention also provides a method of predicting the sensitivity of a subject's cancer or tumor to MDM2 inhibitor treatment, in which the method involves a) measuring the levels of expression of at least three or at least four genes selected from the genes listed in FIGS. 1A-1E in a cancer or tumor sample obtained from the subject and b) determining if the cancer or tumor sample has a wild-type TP53 gene.

In an aspect, the invention provides a method for predicting the sensitivity of a subject's cancer or tumor to MDM2i treatment, comprising: a) measuring the levels of expression of genes comprising at least three genes selected from the genes listed in FIGS. 1A-1E in a cancer or tumor sample obtained from the subject; b) scoring the levels of expression obtained in step a) to obtain a subject's sensitivity score; c) measuring the levels of expression of the at least three genes in plurality of cancer or tumor samples, wherein sensitivities to MDM2i treatment of at least a part of the samples are unknown; d) scoring the levels of expression obtained in step c) to obtain a reference score in each sample and determining a threshold based on the distribution of the reference scores; and e) predicting that the subject is sensitive to MDM2i treatment if the subject's sensitivity score is over the threshold and the subject is resistant to MDM2i treatment if the subject's sensitivity score is under the threshold. In a particular embodiment, step e) is predicting that the subject is sensitive to MDM2i treatment if the subject that is predicted as resistant shows an MDM2 overexpression and preferably has wild type TP53 genes. In an embodiment, the MDM2 overexpression may be caused by an amplification of MDM2 gene in the genome of the subject. In an embodiment, steps b) and d) comprise summing the normalized scores of the levels of the gene expression to obtain the subject's sensitivity score. In an embodiment, the threshold is determined based on Receiver Operating Characteristic (ROC) plots optionally by conducting leave-one-out cross-validation (LOOCV) analysis. In an embodiment, the threshold is determined from the shape of the distribution of the reference scores, for example, by binalization algorithms such as Otsu's method. In an embodiment, the threshold is determined by Gaussian Mixture model. In an embodiment, the invention provides a method for predicting the sensitivity of a subject's cancer or tumor to MDM2i treatment, comprising performing a plurality of predictions, wherein each prediction comprises the above-mentioned steps a) to d) or steps a) to e), and predicting that the subject is sensitive to MDM2i treatment if the number of the predictions where the subject is predicted as sensitive is more than 50%, more than 60%, more than 70%, more than 80% or more than 90% of the total number of the predictions.

In an aspect, the invention provides a method for predicting the sensitivities of at least a part of subjects' cancers or tumors to MDM2i treatment, comprising: a′) measuring the levels of expression of genes comprising at least three genes selected from the genes listed in FIGS. 1A-1E in all cancer or tumor samples obtained from all of the subjects whose sensitivities to MDM2i treatment are unknown; b′) scoring the levels of expression of the genes obtained in step a) to obtain all of the subjects' sensitivity scores and determining a threshold based on the distribution of the sensitivity scores; and e) predicting that the subjects whose sensitivity scores are over the threshold are sensitive to MDM2i treatment and that the subjects whose sensitivity scores are under the threshold are resistant to MDM2i treatment. In a particular embodiment, step e) is predicting that the subject is sensitive to MDM2i treatment if the subject that is predicted as resistant shows an MDM2 overexpression and preferably has wild type TP53 genes. In an embodiment, the MDM2 overexpression may be caused by an amplification of MDM2 gene in the genome of the subject. In an embodiment, steps b′) comprise summing the normalized scores of the levels of the gene expression to obtain the subject's sensitivity score. In an embodiment, the threshold is determined based on Receiver Operating Characteristic (ROC) plots optionally by conducting leave-one-out cross-validation (LOOCV) analysis. In an embodiment, the threshold is determined from the shape of the distribution of the reference scores, for example, by binalization algorithms such as Otsu's method. In an embodiment, the threshold is determined by Gaussian Mixture model. In an embodiment, the invention provides a method for predicting the sensitivity of a subject's cancer or tumor to MDM2i treatment, comprising performing a plurality of predictions, wherein each prediction comprises the above-mentioned steps a′) and b′) or steps a′), b′) and e), and predicting that the subject is sensitive to MDM2i treatment if the number of the predictions where the subject is predicted as sensitive is more than 50%, more than 60%, more than 70%, more than 80% or more than 90% of the total number of the predictions.

In an aspect, the invention provides a method for treating a subject having a cancer or tumor, comprising: a) assessing the sensitivity of a subject's cancer or tumor to MDM2i treatment by the present method for predicting the sensitivity; and b) if the assessment indicates that the cancer or tumor is sensitive to the MDM2i, administering to the subject an effective amount of an MDM2i to treat the cancer or tumor.

In an aspect, the invention provides a pharmaceutical composition for use in treating a cancer or tumor in a subject, wherein the composition comprises an MDM2i, and wherein the subject is determined as sensitive to the MDM2i treatment by assessing the sensitivity of a subject's cancer or tumor to the MDM2i treatment by the present method for predicting the sensitivity.

In an aspect, the invention provides a method of treating a subject having a cancer or tumor, in which the method comprises: a) assessing the sensitivity of a subject's cancer or tumor to MDM2 inhibitor treatment, comprising measuring the levels of expression of at least three or at least four genes selected from the genes listed in FIGS. 1A-1E in a cancer or tumor sample obtained from the subject; b) determining if the cancer or tumor has a wild-type TP53 gene; and c) administering to the subject an effective amount of an MDM2 inhibitor to treat the cancer or tumor, if the assessment of step a) indicates that the cancer or tumor is sensitive to the MDM2 inhibitor and the cancer or tumor specimen has a wild-type TP53 gene.

In each of the above methods of the invention, the genes selected from the genes listed in FIGS. 1A-1E can include some, e.g., at least three or at least four, or all of the genes listed in FIGS. 1A-1E. Alternatively, the genes selected from the genes listed in FIGS. 1A-1E include at least three, at least four, or all of the genes: BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC. Alternatively, the genes selected from the genes listed in FIGS. 1A-1E include at least three, or all, of the genes MDM2, CDKN1A, ZMAT3, DDB2, FDXR, RPS27L, BAX, RRM2B, SESN1, CCNG1, XPC, TNFRSF10B and AEN. Alternatively, the genes selected from the genes listed in FIGS. 1A-1E include at least three, or all, of the genes RPS27L, FDXR, CDKN1A and AEN (and optionally MDM2). In embodiments of the methods, the expression levels of at least 3, 4, 5, 10, 15, 20, 25, 30, 35, 40, 45, 50 or more genes contained in the described gene signatures is predictive of sensitivity of a cancer or tumor sample or cell to an MDM2i.

In an embodiment of the above methods, measuring the levels of expression of the genes involves measuring the level of expression of mRNA. In an embodiment of the methods, measuring the levels of expression of the genes involves measuring the levels of expression of the proteins encoded by the genes. In an embodiment of the methods, expression levels of the genes are measured as increased expression levels of the genes relative to a control.

In an aspect of the invention, the MDM2i is a small molecule chemical compound as further defined herein. In an embodiment, the MDM2i compound functions by targeting MDM2 and inhibiting the interaction of the MDM2 and p53 proteins. In other embodiments, the MDM2i may be a biologic, such as an antibody, e.g., a monoclonal antibody, a polypeptide, a peptide, or a ligand, or a nucleic acid effector of MDM2 function. MDM2 inhibitors suitable for use will preferably inhibit, block, disrupt, or interrupt, either directly or indirectly, the interaction between the MDM2 and p53 proteins.

In a more particular aspect of the invention, the MDM2i is Compound A: [(5R,6S)-5-(4-Chloro-3-fluorophenyl)-6-(6-chloropyridin-3-yl)-6-methyl-3-(propan-2-yl)-5,6-dihydroimidazo[2,1-b][1,3]thiazol-2-yl][(2 S,4R)-2-{[(6R)-6-ethyl-4,7-diazaspiro[2.5]oct-7-yl]carbonyl}-4-fluoropyrrolidin-1-yl]methanone) and salts thereof (See, Example 6 of WO 2010/082612 and Example 6 of U.S. Pat. No. 8,404,691); or Compound B: (3′R,4′S,5′R)—N-[(3R,6S)-6-carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrolidine-3′,3″-indole]-5′-carboxamide) and salts thereof (See, Example 70 of WO 2012/121361 and Example 70 of US Patent Application Publication No. 2012/0264738A). The MDM2i can also be a spirooxindole derivative, an indole derivative, a pyrrolidine-2-carboxamide derivative, a pyrrolidinone derivative, an isoindolinone derivative, or an imidazothiazole derivative. Alternatively, in the above methods, the MDM2i is CGM097, RG7388, MK-8242 (SCH900242), MI-219, MI-319, MI-773, MI-888, Nutlin-3a, RG7112 (R05045337), TDP521252, TDP665759, PXN727, or PXN822, as further described herein. Combinations of two or more of these MDM2 inhibitors are also embraced for use in the methods.

In an aspect, the invention also provides a composition which comprises a plurality of nucleic acid probes for detecting three or more, or four or more, genes listed in FIGS. 1A-1E. In an embodiment, the three or more, or four or more, genes listed in FIGS. 1A-1E are all of the genes listed in FIGS. 1A-1E. In an embodiment, the three or more, or four or more, genes listed in FIGS. 1A-1E are BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC. In an embodiment, the three or more, or four or more, genes listed in FIGS. 1A-1E are the genes MDM2, CDKN1A, ZMAT3, DDB2, FDXR, RPS27L, BAX, RRM2B, SESN1, CCNG1, XPC, TNFRSF10B and AEN. In an embodiment, the three or more, or four or more, genes listed in FIGS. 1A-1E are the genes RPS27L, FDXR, CDKN1A and AEN (and optionally MDM2). In an embodiment, the plurality of nucleic acid probes comprises an array or a microarray.

In an aspect, the invention provides sets of genetic biomarkers whose differential expression or patterns of expression in a cancer or tumor sample or cells derived therefrom compared with a control correlates with the sensitivity of the cancer or tumor to treatment with an MDM2i. In some of its aspects, the invention provides sets of genetic biomarkers, i.e., the genes or sets of genes that make up the gene signatures, whose expression indicates sensitivity of cancer and tumor cells of an individual to exposure to, or treatment with, an MDM2i, such as the MDM2 inhibitors described herein, and assay platforms for detecting gene biomarker expression in cancer and tumor samples in subjects undergoing testing. In another aspect, the gene signatures of the invention can be used in methods to assess or predict the clinical outcome of a patient undergoing cancer treatment with an MDM2i. Such methods for assessment or prediction of clinical outcome include microarrays, PCR, sets of nucleic acid primers and/or probes, immunohistochemistry, ELISA, etc., for detection of the levels of expression of the genes or gene products of the gene signatures of the invention as described herein.

In some of its aspects, the invention provides sets of genetic biomarkers whose differential expression in a cancer or tumor sample correlates with MDM2i sensitivity, as well as with the status of the p53 tumor suppressor signaling pathway, e.g., a functional p53 protein, in a cancer or tumor of an individual. The expression patterns or levels of the genetic biomarkers detected by the methods of the invention can be used to classify or predict cancers or tumors that will likely be sensitive, or respond, to treatment or exposure to an MDM2i.

In an aspect, the invention provides a gene signature and uses thereof as a diagnostic or prognostic platform for use in conjunction with cancer treatments and therapies involving MDM2 inhibitors, for example, those that ultimately may result in a restoration of p53 function in a subject's tumor and cancer cells. Such a platform can comprise a companion diagnostic (e.g., a diagnostic assay or test in a convenient assayable format, such as a microarray or multiplex arrangement of detectable probes or ligands) slated for clinical use with MDM2i drugs or a particular MDM2i. A companion diagnostic involving one or more unique gene signatures indicative of gene expression profiles in individuals' cancer or tumor samples that are sensitive to the MDM2i provides a determination whether an individual will respond to a given MDM2i and predicts sensitivity of the cancer or tumor to an MDM2i. Thus, a cancer or tumor treatment regimen involving the MDM2i can be tailored to those who are most likely to benefit from its successful or effective use, so as to personalize cancer treatment. In accordance with this aspect, the invention further provides methods of treating, diagnosing, or prognosing a subject's cancer or tumor sensitivity to an MDM2i, or sensitivity to treatment with an MDM2i, by assaying or testing a cancer or tumor sample from the subject for the expression of genes within the gene signatures of the invention and thereafter administering an MDM2i to the subject if differential expression of the genes within the MDM2i gene sensitivity signature relative to a control is detected.

As will be appreciated by one skilled in the art, a suitable control will depend on the type of sample, e.g., isolated tumor cells or tumor tissue biopsy sample, and/or assay performed. Without limitation, a control can include assay of normal or non-cancer cells, or cells that are resistant to an MDM2i; a control can also include normalization to one or more constitutively-expressed genes, such as housekeeping genes, whose expression is not affected by an MDM2i, or global normalization of expression of genes of the gene signature against a larger population or number of assayed genes. As is appreciated by the skilled practitioner in the art, normalization, particularly for microarray assay platforms, is conventionally performed to adjust for effects arising from variation in the microarray technology, rather than from biological differences between the samples, such as RNA samples, or between the addressable probes. In general, global normalization in microarray or GENECHIP® technologies provides a solution for adjusting for errors that effect entire arrays by scaling the data so that the average measurement is the same for each array (and each color). Scaling is typically accomplished by computing the average expression level for each array, calculating a scale factor equal to the desired average, divided by the actual average, and multiplying every measurement from the array by that scale factor. The desired average can be arbitrary, or it may be computed from the average of a group of arrays.

In an aspect, the invention provides methods for assessing the expression of genes in the MDM2i inhibitor sensitive gene signatures in cancer and tumor cells derived or cultured from cancer and tumor samples of a subject undergoing testing. The invention also relates to the determination of a sensitivity score, based upon the expression levels of genes within the gene signature, which indicates a cancer or tumor sample's or cell's degree or level of sensitivity to an MDM2i as further described infra. In an embodiment, the method of assessing gene expression is an array or microarray format. In additional aspects, the invention provides a use of the gene signatures indicative of MDM2i sensitivity for predicting the sensitivity of a patient's cancer or tumor to treatment with an MDM2i, in which differential expression levels of some or all of the genes in the gene signatures as described herein measured or detected in a cancer or tumor sample relative to a control predicts the sensitivity of the cancer or tumor to the MDM2i.

In another aspect, the invention provides methods of predicting sensitivity to MDM2i treatment or prognosing the likelihood of successful treatment with an MDM2i of a subject with a cancer or tumor, wherein a cancer or tumor sample obtained from the subject is determined to exhibit differential expression of at least 3-5, 6-10, 11-20, 21-30, 31-40, 41-50, 51-60, 61-70, 71-80, 81-90, 91-100, 101-120, 121-130, 131-140, 141-150, 151-160, 161-170, 171-180 of the genes listed in FIGS. 1A-1E, or a subset thereof, compared to a control. In an embodiment, the subset of genes is at least 3-5, 6-10, 11-20, 21-30, 31-40 of the genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC. In an embodiment, the subset of genes is at least 3 or all of the gene MDM2, CDKN1A, ZMAT3, DDB2, FDXR, RPS27L, BAX, RRM2B, SESN1, CCNG1, XPC, TNFRSF10B and AEN. In an embodiment, the subset of genes is at least 3 or all of the genes RPS27L, FDXR, CDKN1A and AEN (and optionally MDM2). In a related aspect, the invention provides administering to the subject an MDM2i in an amount effective to treat the cancer or tumor, if the practice of the method predicts that the cancer or tumor is sensitive to an MDM2i. In an embodiment, the method includes a comparison of results obtained from the cancer or tumor sample undergoing gene expression evaluation with results obtained from normal, non-cancer, or non-tumor, or MDM2i resistant cells that are evaluated in parallel, or for which an MDM2i gene sensitivity score has been determined, as a control. In another embodiment, a variety of cancer or tumor samples can be evaluated for sensitivity to an MDM2i and the cells' gene signature genes whose expression levels correlate with sensitivity to an MDM2i can be ranked for their degree or level of expression relative to a control.

In an aspect of the invention, a gene signature expression profile can be prepared directly from patients' tumor samples or specimens, for example, by extracting or isolating nucleic acid, such as RNA (mRNA), or encoded protein, directly from the tumor samples or specimens (e.g., biopsied samples and specimens) and assaying for the differential expression of genes in the gene signatures, or proteins encoded therefrom. A determination of differential expression of the gene signature genes, or encoded protein, compared to a control is indicative of MDM2i sensitivity of the samples. In an aspect, if a patient's cancer sample or specimen comprises cells that are amenable to culture, the cells may be enriched or expanded in culture and thereafter may undergo analysis to determine a gene signature profile. The resulting gene signature expression profile, whether prepared directly from a patient's cancer or tumor specimen or prepared from cells derived or cultured therefrom, contains transcript levels (or “expression levels”) of genes in the gene signatures of the invention, or encoded proteins thereof, that predict sensitivity of a cancer or tumor to an MDM2i. In some embodiments, differential expression of the genes in the gene signature is increased expression relative to a control and indicates that the cancer or tumor is sensitive to an MDM2i.

In an aspect of the invention, the expression of genes in gene signatures indicative of MDM2i sensitivity can be predictive of the sensitivity of a particular type of cancer, such as, for example, leukemia, lymphoma, melanoma, or myeloma, and others as described herein, to treatment with an MDM2i drug and/or for a particular course of treatment with the MDM2i drug. The expression of genes in gene signatures indicative of MDM2i sensitivity can also be predictive of an individual's survival, or duration of survival, a pathological complete response (pCR) to treatment, or another measure of the individual's treatment outcome with an MDM2i, such as progression free interval, or tumor size, volume, and the like. As described and exemplified herein, the MDM2i gene sensitivity signatures have been identified in a large number of cancer cell lines by correlating the level of in vitro sensitivity to MDM2i with levels of expression of particular genes in the cancer cells. Expression profiles of genes in the gene signatures yielded sensitivity scores as applied in in vivo tumored animal model experiments described in the Examples herein.

In an aspect, the invention provides methods of diagnosing, prognosing, and/or treating a subject in need thereof involving testing or assaying a subject's cancer or tumor sample, or cells derived therefrom, for the expression of genes in a gene signature of the invention that is indicative of MDM2i sensitivity. Preferably, gene expression is detected as mRNA production, but protein expression may also be detected. Subjects whose cancers or tumors differentially express some or all of the genes of the gene signatures of the invention are considered to be sensitive to, and treatable with, an MDM2i. In various embodiments, the expression of at least three, at least four, or all, of the genes in the gene signatures is detected in the test or assay and is predictive of MDM2i sensitivity. In an aspect, the methods of diagnosis, prognosis, and/or treatment which involve assaying a subject's cancer or tumor sample for expression of the genes within the MDM2i gene sensitivity signatures of the invention, further involve determining if the cancer or tumor sample has a wild-type TP53 gene.

In another of its aspects, the invention provides a kit containing reagents for the detection of at least three or at least four genes listed in FIGS. 1A-1E, which are indicative of sensitivity to an MDM2i, and instructions for use.

In another aspect, the invention provides a kit for predicting sensitivity of a cancer or tumor sample to an MDM2i, wherein the kit comprises nucleic acid probes that specifically bind to nucleotide sequences corresponding to genes listed in FIGS. 1A-1E, and a means for labeling the nucleic acids.

In another aspect, the invention provides a kit for predicting sensitivity of a cancer or tumor sample to an MDM2i, wherein the kit comprises antibodies or ligands that specifically bind to polypeptides or peptides encoded by at least three or at least four genes listed in FIGS. 1A-1E, and a means of labeling the antibodies or ligands that specifically bind to the polypeptides or peptides encoded by the genes.

In each of the above kits according to the invention, the at least three or the at least four genes listed in FIGS. 1A-1E can be all of the genes listed in FIGS. 1A-1E. Alternatively, in each of the kits, the at least three, at least four, or all, of the genes listed in FIGS. 1A-1E are BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC. Alternatively, in each of the kits, the at least three or at least four genes listed in FIGS. 1A-1E are the genes MDM2, CDKN1A, ZMAT3, DDB2, FDXR, RPS27L, BAX, RRM2B, SESN1, CCNG1, XPC, TNFRSF10B and AEN. Alternatively, in each of the kits, the at least three or at least four genes listed in FIGS. 1A-1E are the genes RPS27L, FDXR, CDKN1A and AEN (and optionally MDM2).

In each of the above kits according to the invention, the MDM2i is Compound A and salts thereof or Compound B and salts thereof as described herein. In other aspects, the MDM2i in the kits is a spirooxindole derivative, an indole derivative, a pyrrolidine-2-carboxamide derivative, a pyrrolidinone derivative, an isoindolinone derivative, or an imidazothiazole derivative. Alternatively, in each of the above kits, the MDM2i can be Compound A and salts thereof, Compound B and salts thereof, CGM097, RG7388, MK-8242 (SCH900242), MI-219, MI-319, MI-773, MI-888, Nutlin-3a, RG7112 (R05045337), TDP521252, TDP665759, PXN727, or PXN822, as described herein. Combinations of one or more of the MDM2i compounds are embraced by the kits of the invention.

The foregoing and other aspects, features and advantages of the invention and its embodiments will become apparent in the descriptions of the accompanying drawings and in the embodiments provided herein.

### BRIEF DESCRIPTION OF DRAWINGS

FIGS. 1A-1E present in tabular format 177 gene signature biomarkers that are differentially expressed in cancer or tumor samples or cells that are sensitive to the MDM2i, Compound A, as described herein. The table presented in FIGS. 1A-E shows the gene identification or GenBank number (“Reporter”); the gene name (“gene”); p value, a well-known measure of statistical significance in terms of false positive rate for each test gene and associated with the two-class Student's t-Test; q value, a well-known measure of statistical significance in terms of the false discovery rate for multiple testing hypothesis (See, e.g., J. D. Storey et al., 2003, Statistical significance for genome-wide studies, Proc. Natl. Acad. Sci. USA, 100(16):9440-45); and t-Test value (“tStatistic”), resulting from application of the Student's two-class t-Test for each test sample.

FIG. 2 depicts the characterization of response phenotypes resulting from the cell line analysis of MDM2 sensitivity or resistance as described in Example 2. Cell lines were ranked by IC₅₀ value and were designated as “S” for sensitive, “M” for moderate, and “R” for resistant to MDM2i treatment. IC₅₀ value indicated two general response phenotypes, S and R, with moderate responders therebetween.

FIGS. 3A-3F show the MDM2i gene sensitivity signature score or value of each cell line, as obtained from the analysis of 177 genes (i.e., the genes presented in FIGS. 1A-1E), 175 genes (i.e., the genes presented in FIGS. 1A-1E, except for EDA2R and SPATA18), 40 genes (i.e., the genes presented in Table 1 herein), 4 genes (i.e., RPS27L, FDXR, CDKN1A and AEN), and 3 genes (i.e., RPS27L, FDXR and CDKN1A).

FIG. 4 shows the results of the prediction using samples whose sensitivities to MDM2i treatment are unknown as training sets. The prediction models A to E are detailed in Examples. In the prediction model F, thresholds were determined as the value of the score where the distribution of the scores formed a valley in a histogram. In the prediction model G, thresholds were determined as the value of the first quartile of the averaged z-scores among TP53 wild type samples.

FIG. 5 shows the results of the prediction of sensitivity in melanoma cell lines using samples whose sensitivities to MDM2i treatment are unknown as training sets. In the “score in wt” prediction model, thresholds were determined as the value of the first quartile of the averaged z-scores among TP53 wild type samples. In the “score distribution” prediction model, thresholds were determined, by Otsu's method, as the value of the score where the distribution of the scores formed a valley in a histogram.

FIG. 6 shows the results of the prediction of sensitivity in Pdx models using samples whose sensitivities to MDM2i treatment are unknown as training sets. The “score in wt” and the “score distribution” prediction models are explained above.

FIG. 7 shows the effect of p53 mutation rates in training sets on the distribution of the optimized thresholds in training sets using samples whose sensitivities to MDM2i treatment are unknown as training sets.

FIG. 8 shows that the sensitivity and specificity of the prediction do not change regardless of p53 mutation rates in training sets.

FIG. 9 shows that some of the cell lines with MDM2 gene amplification on the genome affect the prediction accuracy.

FIG. 10 shows that the level of expression of MDM2 improves the prediction accuracy.

FIG. 11 shows that the sensitivity to MDM2i treatment can be accurately predicted in various training set sizes.

FIGS. 12A to 12K show a list of all the cell lines of CCLE datasets analyzed in Examples with their p53 mutation status and sensitivities to MDM2i treatment. “M” and “W” in the TP53 column represent “mutant” and “wild type”, respectively. “S” and “R” in the S/R column represent “sensitive” and “resistant”, respectively.

FIGS. 13A and 13B show a list of PDx models analyzed in Examples with their p53 mutation status and sensitivities to MDM2i (Compound B) treatment. “M” and “W” in the TP53 column represent “mutant” and “wild type”, respectively. “S” and “R” in the S/R column represent “sensitive” and “resistant”, respectively.

### DETAILED DESCRIPTION

The invention relates to the discovery of gene signatures containing genes, and sets of genes, whose expression in cancers and tumors is predictive of the sensitivity of the cancers and tumors to MDM2 inhibitors and other compounds or agents having similar activity. As used in the methods of the invention, the gene signatures provide biomarkers, or sets of biomarkers, whose expression indicates the sensitivity of cancer and tumor samples, and cells derived therefrom, to an MDM2i and to MDM2i treatment. As used herein, the term “indicates” also may be used interchangeably with the terms “corresponds to”, “is correlated or associated with”, or “is predictive of”.

Gene signatures are generally important and powerful molecular tools that can reveal, at the molecular level, a variety of biologically and clinically relevant characteristics of biological samples. A gene signature can be considered to embrace a particular set of gene biomarkers. More specifically, gene signatures are provided that contain genes whose expression can indicate sensitivity to clinically significant drugs, such as MDM2 inhibitors as described herein, that are used in the treatment of different cancer and tumor types and subtypes. The gene signatures can be further utilized to predict likely clinical responses or outcomes in treating patients having a cancer or tumor with MDM2i drugs.

A basic characteristic of the gene signatures provided by and used according to the methods of the invention is the identification of genes, or sets of genes, whose expression patterns in a tumor or cancer sample or specimen allow a determination of the sensitivity of the tumor or cancer to an MDM2i, or other, similarly-acting compound. The gene signatures of the invention comprise those genes that are expressed, e.g., show differential expression, in cells that are sensitive to an MDM2i compound, drug, or combination thereof. In an embodiment, the invention provides gene signatures related to the sensitivity or response of cancers to treatment with a small molecule, low molecular weight MDM2i. Such gene sensitivity signatures also serve as genetic biomarkers for use in conjunction with MDM2i treatment and therapy, for example, to assess, determine, diagnose, predict, or prognose the sensitivity of an individual's cancer to treatment with an MDM2i. The gene signatures indicative of MDM2i sensitivity according to the invention were determined by analyzing the differential expression of genes in a large number of cancer or tumor derived cell lines that had been exposed to MDM2 inhibitors, as described further in the Examples herein.

### Terms and Definitions

The technical and scientific terms used herein are intended to have meanings that are commonly and conventionally known to those having skill in the art to which the described invention pertains, unless otherwise indicated. Such terms encompass methods, processes, procedures, reagents, devices, biological molecules and compounds that are known and practiced in the art. The definition and explanation of terms herein are not meant to be exhaustive or limiting, but are instead provided to facilitate the review of various aspects and embodiments of the described invention.

The term “MDM2” refers to an E3 ubiquitin ligase which can interact with p53 and cause p53 degradation. MDM2 as used herein includes, but not limited to, mouse MDM2 and the human ortholog of MDM2 (also called “Human Double Minute 2” or “HDM2”).

The term “MDM2 inhibitor” refers to an inhibitor inhibiting MDM2 functions or activities on p53 degradation. It will be understood that the term “an MDM2i” may embrace “one or more MDM2 inhibitors” or “a combination of MDM2 inhibitors” herein.

The term “array” as used herein refers to an arrangement, typically an ordered arrangement, of biological molecules, e.g., nucleic acids, polypeptides, peptides, biological samples, placed in discrete, assigned and addressable locations on or in a surface, matrix, or substrate. Microarrays are miniaturized versions of arrays that are typically evaluated or analyzed microscopically. Nucleic acid, e.g., RNA or DNA, arrays are arrangements of nucleic acids (such as probes) in assigned and addressable locations on a solid surface or matrix. Nucleic acid arrays encompass cDNA arrays and oligonucleotide arrays and microarrays; they may be referred to as biochips, or DNA/cDNA chips. Microarrays, as well as their construction, reagent components and use are known by those having skill in the pertinent art. By way of example, microarray technology useful for determining and measuring gene expression status is provided in US 2011/0015869.

The term “biomarker” generally refers to a gene, an expressed sequence tag (EST) derived from the gene, a set of genes, or a set of proteins or peptides whose expression levels change under certain conditions, or differ in certain cellular contexts, such as in cells sensitive to MDM2 inhibitors as opposed to those that are insensitive to MDM2 inhibitors. In general, when the expression levels of the genes or gene sets correspond to a certain condition, the gene(s) serve(s) as one or more biomarkers for that condition. Biomarkers can be differentially expressed among individuals, (e.g., those with a cancer or tumor type) according to prognosis and disease state; thus, biomarkers may be predictive of different survival outcomes, as well as of the benefit drug susceptibility and sensitivity.

The term “binding” refers generally to an interaction or association between two substances or molecules, such as the hybridization of one nucleic acid molecule to another (or to itself); the association of an antibody with a polypeptide, protein, or peptide; or the association of a protein with another protein or nucleic acid molecule. An oligonucleotide molecule binds or stably binds to a target nucleic acid molecule if a sufficient amount of the oligonucleotide molecule forms base pairs or is hybridized to its target nucleic acid molecule, to permit detection of that binding. Preferentially, binding refers to an association in which one molecule binds to another with high affinity, and binds to heterologous molecules at a low affinity. Binding can be detected by any procedure known to one skilled in the art, such as by physical or functional properties of the target/oligonucleotide complex. For example, binding can be detected functionally by determining whether there is an observable effect upon a biosynthetic process, e.g., expression of a gene, DNA replication, transcription, translation, etc.

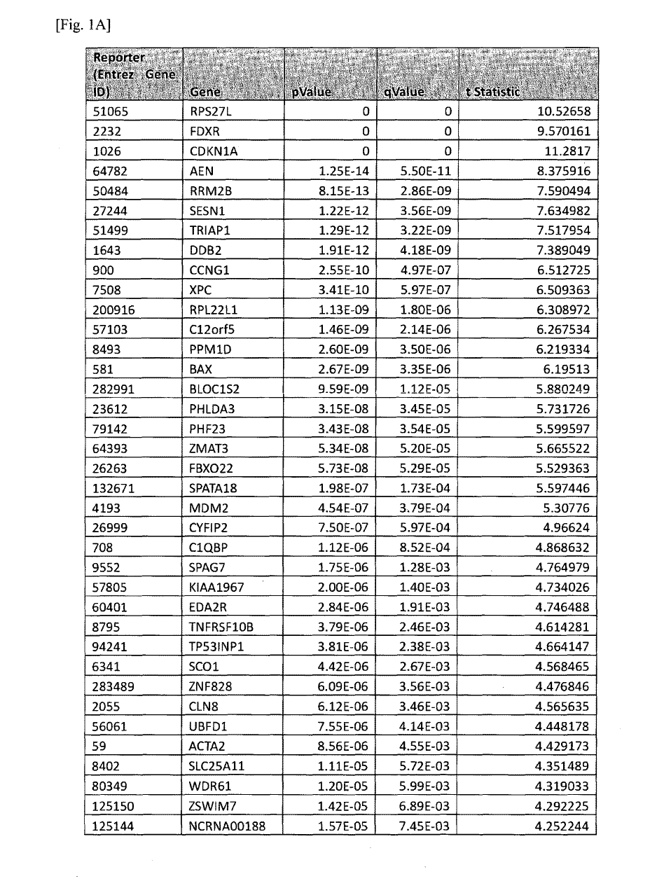

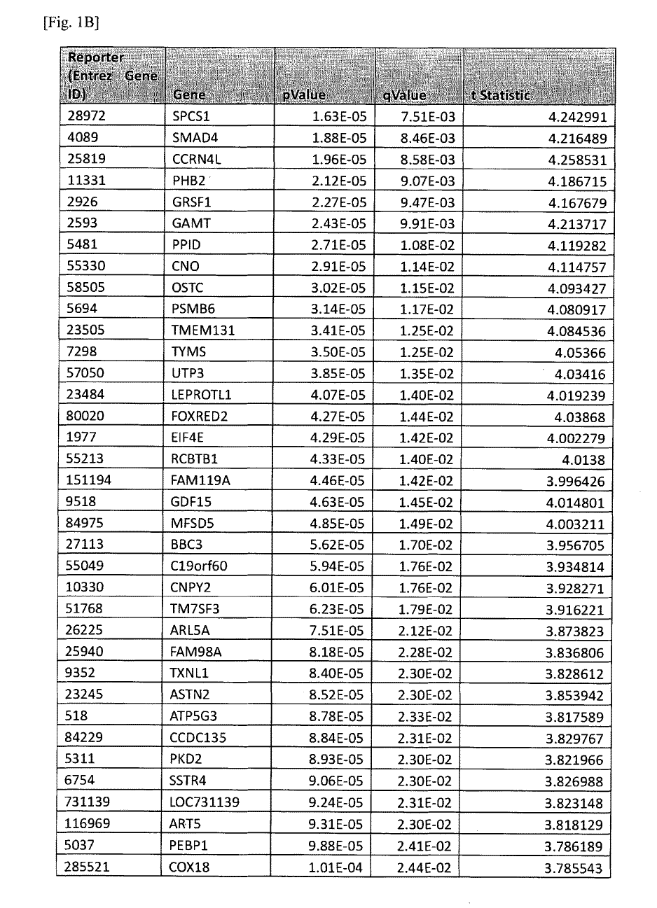

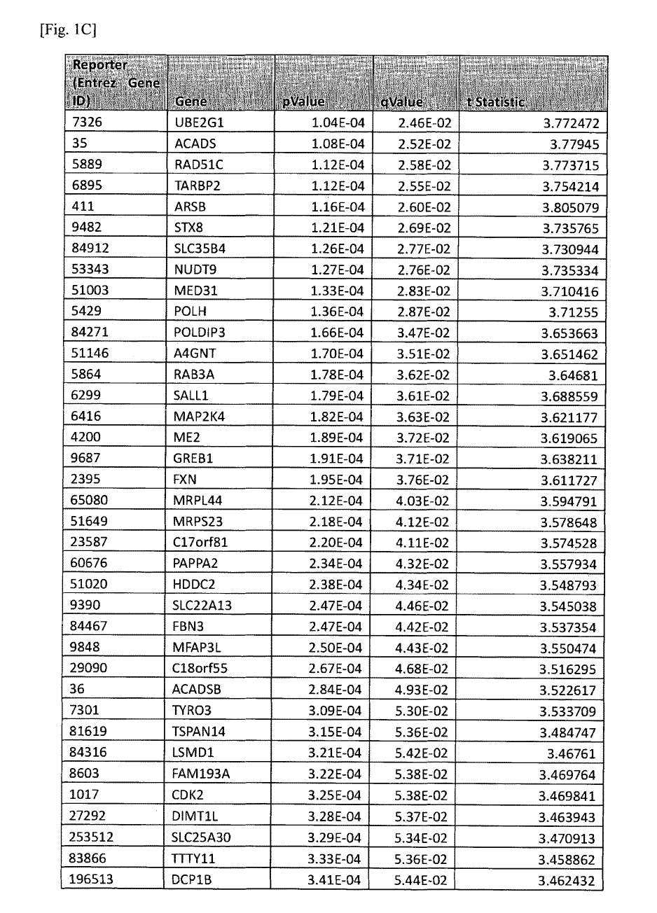

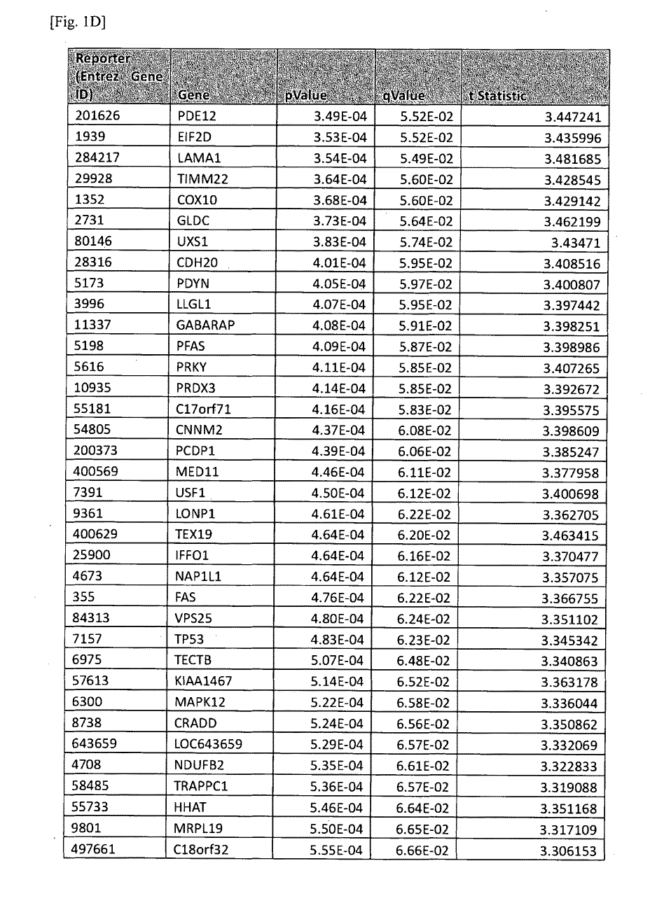

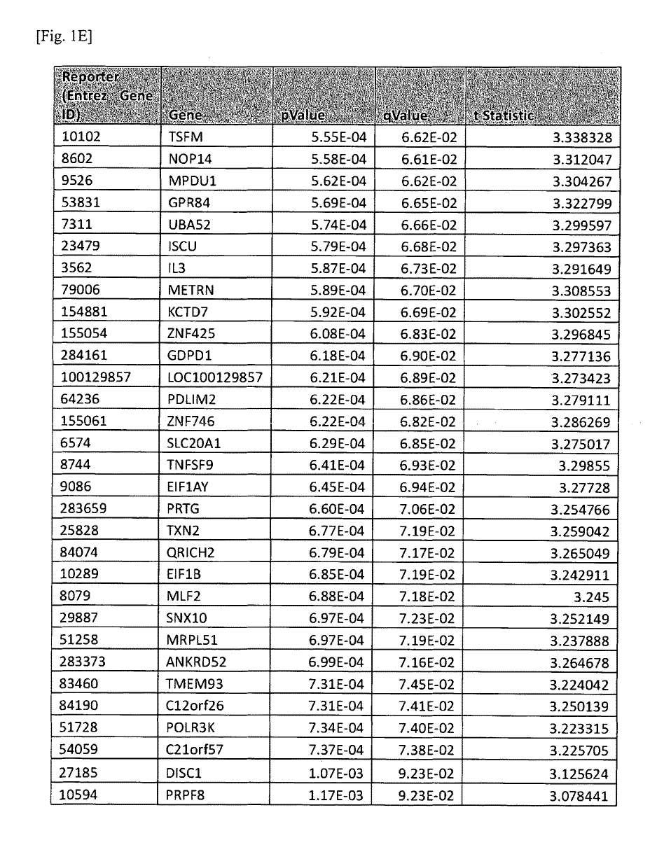

The term “gene” as used herein refers to a DNA sequence which is expressed in a sample as an RNA transcript; a gene can be a full-length gene (protein encoding or non-encoding) or an expressed portion thereof, such as expressed sequence tag or “EST.” Thus, the genes listed in FIGS. 1A-1E and in Table 1 and elsewhere herein as components of the gene signatures of the invention are each independently a full-length gene sequence, whose expression product is present in samples, or is a portion of an expressed sequence, e.g., EST sequence, that is detectable in samples. The genes listed in FIGS. 1A-1E and the sequences thereof, which are incorporated by reference herein, are found in the publicly available GenBank database by virtue of their gene identification names or Entrez Gene ID designations as provided in the figure. Accordingly, all GenBank identification numbers and sequences related thereto are incorporated by reference in their entirety herein.

The terms “gene signature”, “gene expression signature” and “gene sensitivity signature” are used interchangeably herein as they refer to the expression, such as differential expression, or the expression patterns, of genes predictive of cellular response in cancers or tumors sensitive to an MDM2i in accordance with the invention. For example, in an embodiment, tumor or cancer samples showing sensitivity to an MDM2i have increased or elevated levels of expression of genes contained in the gene signatures of the invention compared with a control.

As used in accordance with the present invention, “gene expression” means the process of converting genetic information encoded in a gene into RNA (e.g., mRNA, rRNA, tRNA, or snRNA) through transcription of the gene (e.g., as mediated by the enzymatic action of an RNA polymerase), and for protein-encoding genes, into protein through “translation” of mRNA. Gene expression can be regulated at any point in the pathway leading from DNA to RNA to protein. The regulation of gene expression can include controls on transcription, translation, RNA transport and processing, as well as degradation of intermediary molecules such as mRNA. Regulation can also involve activation, inactivation, compartmentalization, or degradation of specific protein molecules after they are produced. The expression of a nucleic acid molecule can be altered relative to a normal or wild type nucleic acid molecule.

Alterations in gene expression, such as differential expression, can include, without limitation, overexpression, increased expression, underexpression, or suppressed expression, as compared to a control, such as non-cancer cells or in relation to normalized expression levels. Alterations in the expression of a nucleic acid molecule may be associated with, and in some instances cause, a change in expression of the corresponding protein. Illustratively, gene expression can be measured to determine differential expression of genes in the gene signatures indicative of MDM2i sensitivity of a subject's cancer or tumor sample in order to predict the subject's likelihood of responding to MDM2i treatment for the purpose of administering an MDM2i to the subject, and/or personalizing an effective treatment with an MDM2i, and/or predicting the subject's survival time.

An increase in expression, which may also be referred to as upregulated or activated expression, used in reference to a gene or nucleic acid molecule, refers to any process that causes or results in increased or elevated production of a gene product, such as all types of RNA, or protein. Increased or elevated gene expression includes any process that increases the transcription of a gene or the translation of mRNA into protein. Increased (or upregulated) gene expression can include any detectable or measurable increase in the production of a gene product. Illustratively, the production of a gene product, (such as at least three, at least four, or all, of the genes of FIGS. 1A-1E; or at least three, at least four, or all, of the gene signature genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC; or at least three, or all, of the gene signature genes MDM2, CDKN1A, ZMAT3, DDB2, FDXR, RPS27L, BAX, RRM2B, SESN1, CCNG1, XPC, TNFRSF10B and AEN; or at least three, or all, of the gene signature genes RPS27L, FDXR, CDKN1A and AEN (and optionally MDM2)), is increased by a measurable, relative amount, for example, and without limitation, an increase of at least 1.5-fold, at least 2-fold, at least 3-fold, at least 4-fold, at least 5-fold, or at least 6-10 fold, as compared to a control. The control may be the amount of gene expression in a biological sample, such as a normal cell, or a reference value, or a normalized value of cellular gene expression. In an example, a control is the relative amount of gene expression in a biopsy of the same tissue type from a subject who does not have a tumor, as does the subject in question (who is undergoing testing). In another example, a control is the relative amount of gene expression in a tissue biopsy from non-tumored tissue of the same tissue type as that of the tumor, taken from the subject having the tumor and undergoing testing.

Alternatively, decrease in expression, which may also be referred to as downregulated expression, used in reference to a gene or nucleic acid molecule, refers to any process that causes or results in decreased production of a gene product, such as all types of RNA or protein. Decreased or downregulated gene expression typically includes processes that cause or result in a decrease of gene transcription or translation of mRNA into protein. Gene downregulation includes any measurable or detectable decrease in the production of a gene product, for example, and without limitation, a decrease of at least 1.5-fold, at least 2-fold, at least 3-fold, at least 4-fold, at least 5-fold, or at least 6-10 fold, as compared to a control, e.g., the amount of gene expression in a normal cell, or a reference value.

The term “cancer” as used herein is understood to encompass neoplasms and tumors, which refer to abnormal growths or abnormally growing cells that can invade surrounding tissues and spread to other organs, i.e., become malignant, if left untreated. Neoplasms are abnormal growths (or masses) of tissues comprised of cells that form as a result of neoplasia, which is the abnormal growth and proliferation of cells, either malignant or benign. Neoplasms and tumors can include the abnormal growths of precancerous and cancerous cells and tissues, which grow more rapidly than normal cells and that will continue to grow and compete with normal cells for nutrients if not treated. Neoplasms may include, without limitation, solid and non-solid tumors, such as hollow or liquid-filled tumors, and also hematological cell neoplasias or neoplasms, e.g., lymphomas, leukemias and myelomas.

The term “cancer” is also intended to embrace neoplasms and tumors of various origins within and on the body, various types and subtypes, as well as organ, tissue and cell samples and specimens, e.g., biological samples or specimens, thereof. Illustratively, appropriate cancer samples or specimens include any conventional biological samples or specimens, including clinical samples obtained from a human, e.g., a patient undergoing treatment for cancer, or a veterinary subject. A sample may refer to a part of a tissue that is a diseased or healthy portion of the tissue, or to the entire tissue. Tissue samples can be obtained from a subject by employing any method or procedure as known and practiced in the art.

Exemplary samples or specimens include, without limitation, cells, cell lysates, blood smears, cyto-centrifuge preparations, cytology smears, bodily fluids, e.g., peripheral blood, blood, plasma, serum, urine, saliva, sputum, bronchoalveolar lavage, semen, etc., tissue biopsy or autopsy samples or specimens, e.g., neoplasm biopsies, fine-needle aspirates, cell-scraping, surgical specimens, circulating tumor cells (CTCs), and/or tissue sections, e.g., cryostat tissue sections and/or paraffin-embedded tissue sections. In some cases, the sample includes systemic or circulating tumor or neoplasm cells. In certain examples, a tumor or neoplasm sample is used directly, e.g., fresh or frozen, or can be manipulated prior to use, for example, by fixation, e.g., using formalin, and/or embedding in wax, such as formalin-fixed or paraffin-embedded tissue samples. A sample may contain genomic DNA, RNA (including mRNA), protein, or combinations thereof, etc., obtained from a subject. In a preferred embodiment, the sample contains mRNA to allow the analysis of expression levels of the genes within the gene signature.

The term “control” typically refers to a sample, reference, or standard that is used as a basis for comparison with one or more experimental or test samples. In the instant case, an experimental sample can comprise a tumor specimen or sample obtained from an individual treated with or to be treated with an MDM2i. In some cases, the control is a sample that is obtained from a healthy, non-tumored individual; in some cases, the control is a non-tumor tissue sample taken from the individual having the tumor treated with, or to be treated with, the MDM2i. In other cases, the control can be a standard reference value, or a range of values, or a historical control. By way of example, a standard range of values may be obtained from a previously tested control sample, e.g., a group of samples that represent baseline or normal values, such as the levels of the genes of an MDM2i gene sensitivity signature in non-tumor tissue; or a previously-tested group of individuals whose tumors are sensitive to MDM2i; or a previously-tested group of individuals whose tumors are insensitive to MDM2i. In addition, controls that can serve as standards of comparison to a test sample for the determination of differential gene expression include samples that are believed to be normal, i.e., not altered for the desired characteristic, such as from a subject who does not have a cancer or tumor. A range of values, such as laboratory values or values obtained from in vitro experiments, may also be used as controls, although such values are often established based on locally determined laboratory conditions and may be subject to somewhat more variability. In addition and without limitation, a control can be a relative amount of gene expression in a biological sample, or test population, and can also embrace normalization, for example, global normalization to the expression levels of all genes within a DNA array as discussed further herein, or normalization to expression levels of one or more internal control genes that are constitutively expressed, e.g., so-called “housekeeping genes”, and that exhibit constant expression levels in most, if not all, types of cells, as understood by one having skill in the pertinent art.

Housekeeping genes are typically constitutive genes that are required for the maintenance of basic cellular function and are expressed in all cells of an organism under normal and pathophysiological conditions. Optimally, housekeeping genes are expressed at relatively constant levels in most non-pathological situations and their expression does not vary significantly under differing experimental conditions. Examples of such housekeeping genes include, without limitation, actin (β-actin; RefSeq ID: NM_001101.3), glucuronidase (GUS; RefSeq ID: NM_000181.3), transferrin receptor (TFRC; RefSeq ID: NM_001128148.1), glyceraldehyde-3-phosphate dehydrogenase (G3PDH; RefSeq ID: NM_002046), hypoxanthine phosphoribosyltransferase 1 (HPRT1; RefSeq ID: NM_000194), peptidylprolyl isomerase (PPIA; RefSeq ID NM_021130.3), 18s rRNA (RefSeq ID: NR 003286.2), and the like. In other examples, the control includes the expression levels of one or more housekeeping genes, such as albumin, tubulin, cyclophilin, L32, and 28S rRNA, as described, for example, in O. Thellin et al., 1999, J. Biotechnol., 75(2-3):291-5.

In some cases, expression levels of the disclosed genes (such as expression of at least three, at least four, at least five, at least six, at least ten, or all, of the genes listed in the gene signatures of FIGS. 1A-1E; in Table 1; or at least three, or all, of the genes in the gene set RPS27L, FDXR, CDKN1A and AEN (and optionally MDM2)) are normalized relative to the expression levels of one or more housekeeping genes, e.g., in the same or different cancer or neoplasm sample. An aggregate value is obtained in some cases by calculating the level of expression of each of the genes (e.g., each of the genes in a gene signature) and using a positive or negative weighting for each gene depending on whether the gene is positively or negatively regulated by a condition (e.g., sensitivity to MDM2i treatment or a survival risk score). In some cases, the normalized expression of the gene or the gene signature, or an aggregate value, is determined to be increased or decreased relative to the median normalized expression of the gene or gene signature, or to an aggregate value, for a set of cancers or cancer types. In some cases, the median normalized expression or aggregate value is obtained from publicly-available microarray datasets, such as leukemia, lymphoma, melanoma, or myeloma cancer microarray datasets. In an example, a median normalized expression or aggregate value for expression genes of the gene signature is determined using microarray datasets.

In some cases, a score (sensitivity score) is calculated from the normalized expression level measurements. The score can be utilized to provide cutoff points or values to identify various parameters, such as a cancer or tumor as being sensitive, or less likely to be sensitive, to an MDM2i and/or low, medium, or high sensitivity of a subject with a cancer or tumor to MDM2i treatment or therapy. In some cases, the cutoff points are often determined using training and validation datasets. In other cases, the cutoff points are determined using only training datasets without MDM2i sensitivity data. By way of example, a supervised approach can be utilized to establish the cutoff that distinguishes those who will be sensitive (responders) from those who will not respond to MDM2i treatment, for example, by comparing gene signature expression in responders and non-responders. In another example, an unsupervised approach can be utilized to determine empirically a cutoff level (for example, top 50% versus bottom 50%, or top tercile versus bottom tercile) that is predictive of an outcome, i.e., sensitivity to MDM2i treatment. The cutoff determined in the training set can be tested in one or more independent validation datasets.

The term “diagnose” refers to the recognition or identification of a disease or condition by signs or symptoms, frequently involving the use of external tests, evaluations and analyses. A diagnosis of the disease or condition results from the entirety of the procedures involved in making and drawing a conclusion to identify the disease or condition. According to the invention, the sensitivity of a patient's cancer or tumor to an MDM2i, as well as the likelihood that the patient will respond to MDM2i treatment, can be diagnosed by the practice of the described methods in which the expression levels of genes within the gene signatures are measured. In various embodiments, the expression levels of at least three, at least four, or all, of the genes of FIGS. 1A-1E are measured; or the expression of at least three, or at least four, or all, of the gene signature genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC are measured; or the expression of at least three, or all, of the gene signature genes MDM2, CDKN1A, ZMAT3, DDB2, FDXR, RPS27L, BAX, RRM2B, SESN1, CCNG1, XPC, TNFRSF10B and AEN; or the expression of at least three, or all, of the gene signature genes RPS27L, FDXR, CDKN1A and AEN (and optionally MDM2) are measured. By example, expression of gene signature genes in a subject's cancer or tumor sample undergoing testing and indicative of MDM2i sensitivity serves to diagnose the subject as one whose cancer or tumor will be sensitive to MDM2i treatment.

As used herein, “differentially expressed” refers to a difference or alteration in expression, such as an increase or a decrease, in the conversion of gene-encoded information, (such as a gene associated with MDM2i sensitivity), into RNA (e.g., mRNA), and/or in the conversion of mRNA into protein. In some cases, the difference or alteration is relative to a control or a reference value, or to a range of control or reference values, for example, the average expression of a group or a population of subjects, such as a group of subjects having a good response or a poor response to MDM2i treatment (e.g., MDM2i sensitive versus MDM2i insensitive populations). In some cases, the difference or alteration can be relative to non-tumor tissue from the same subject or a healthy subject. The detection of differential expression can involve measuring a change in gene or protein expression, such as a change in expression of at least three, or at least four of the gene signature genes of FIGS. 1A-1E associated with MDM2i sensitivity.

Detecting the expression of a gene product, as well as detecting the differential expression of a gene product, refer to measuring, or determining qualitatively or quantitatively, the level of expression of nucleic acid or protein in a sample by one or more suitable means as known in the art, e.g., by microarray analysis, PCR (RT-PCR), immunohistochemistry, immunofluorescence, mass spectrometry, Northern blot, Western blot, etc.

The term “MDM2i” encompasses a number of low molecular weight MDM2 inhibitors that are suitable for use according to the invention. More specifically, MDM2 inhibitors include Compound A (See, Example 6 of WO 2010/082612 and Example 6 of U.S. Pat. No. 8,404,691: [(5R,6S)-5-(4-Chloro-3-fluorophenyl)-6-(6-chloropyridin-3-yl)-6-methyl-3-(propan-2-yl)-5,6-dihydroimidazo[2,1-b][1,3]thiazol-2-yl][(2S,4R)-2-{[(6R)-6-ethyl-4,7-diazaspiro[2.5]oct-7-yl]carbonyl}-4-fluoropyrrolidin-1-yl]methanone) and salts and hydrates thereof; and Compound B (See, Example 70 of WO 2012/121361 and Example 70 of US Patent Application Publication No. 2012/0264738A:

(3′R,4′S,5′R)—N-[(3R,6S)-6-carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrolidine-3′,3″-indole]-5′-carboxamide) and salts thereof, including the p toluenesulfonate thereof.

Examples of MDM2 inhibitors targeting the MDM2-p53 binding site have been reported and include spirooxindole derivatives (WO 2006/091646, WO 2006/136606, WO 2007/104664, WO 2007/104714, WO 2008/034736, WO 2008/036168, WO 2008/055812, WO 2008/141917, WO 2008/141975, WO 2009/077357, WO 2009/080488, WO 2010/084097, WO 2010/091979, WO 2010/094622, WO 2010/121995; J. Am. Chem. Soc., 2005, 127, 10130-10131; J. Med. Chem., 2006, 49, 3432-3435; and J. Med. Chem., 2009, 52, 7970-7973); indole derivatives (WO 2008/119741); pyrrolidine-2-carboxamide derivatives (WO 2010/031713); pyrrolidinone derivatives (WO 2010/028862, WO 2010/031713, WO 2011/061139, WO 2011/098398, WO 20120/34954, WO 2012/076513); isoindolinone derivatives (WO 2006/024837; and J. Med. Chem., 2006, 49, 6209-6221); and others (WO 2011/076786, WO 2012/175487, WO 2012/175520, WO 2012/066095 and WO 2011/046771).

Examples of preferred MDM2i compounds for use in accordance with the described invention include Compound A (Example 6 of WO 2010/082612 and Example 6 of U.S. Pat. No. 8,404,691); Compound B (Example 70 of WO 2012/121361 and Example 70 of US Patent Application Publication No. 2012/0264738A); CGM097; RG7388; MK-8242 (SCH900242); MI-219; MI-319; MI-773; MI-888; Nutlin-3a; RG7112 (R05045337), (Y. Yuan et al., 2011, J. Hematol. Oncol., 4:16); a benzodiazepinedione, for example, TDP521252 and TDP665759; and an isoquinolinone, for example, PXN727 and PXN822 (Y. Yuan et al., 2011, J. Hematol. Oncol., 4:16; S. Wang et al., Top Med Chem 8: 57-80, 2012; and Q. Ding et al., J. Med Chem 2013). Other small molecule inhibitors of MDM2-p53 interactions are described, for example, in Y Zhao et al., 2013, BioDiscovery, 8(4):1-15, such as spirooxindole-containing compounds, piperidinone-containing compounds, 1,4-diazepine compounds, or isoindolinone compounds, and salts thereof.

The term “prognosis” refers to the prediction of prospective survival and recovery from a disease or condition, as anticipated from the usual course of that disease or condition, or as indicated by special features presented by a subject. A prognosis can also predict the course of a disease associated with a particular treatment, for example, by determining that a patient will or will be likely to survive for a given period of time, depending on, for example, a patient's response or sensitivity to a given therapy or treatment regimen involving one or more drugs or compounds. Thus, the practice of the methods of the invention in which the sensitivity of a patient's cancer or tumor to an MDM2i is determined by measuring expression levels of genes of the described MDM2i sensitive gene signatures is associated with a prognosis that the patient will respond, or is likely to respond, to MDM2i treatment. In various embodiments, the expression levels of at least three, at least four, or all, of the genes of FIGS. 1A-1E are measured; or the expression of at least three, at least four, or all, of the gene signature genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC are measured; or the expression of at least three, or all, of the gene signature genes MDM2, CDKN1A, ZMAT3, DDB2, FDXR, RPS27L, BAX, RRM2B, SESN1, CCNG1, XPC, TNFRSF10B and AEN; or the expression of at least three, or all, of the gene signature genes RPS27L, FDXR, CDKN1A and AEN (and optionally MDM2) are measured.

As used herein, a “subject” is typically a multi-cellular vertebrate organism, including human and non-human mammals. The term “subject” may be used interchangeably herein with the term individual; frequently, a subject or individual is a patient who is afflicted with a cancer, tumor, neoplasia, or neoplastic condition. Thus, the practice of the invention is suitable for human and veterinary use.

The term “treating” in a general sense refers to achieving or obtaining a desired physiologic and/or pharmacologic effect, whether prophylactic, therapeutic, or both. As used herein “treating” or “treatment” can refer to preventing, inhibiting, curing, reversing, attenuating, alleviating, abrogating, minimizing, suppressing, reducing, diminishing, stabilizing, or eliminating the deleterious effects of a disease state, disease progression, disease causative agent, or other abnormal condition, such as a non-benign or malignant cancer, tumor, or neoplasm. For example, treatment may involve alleviating a symptom, although not necessarily all of the symptoms, of a disease, or attenuating the symptoms or progression of a disease. The treatment of cancer, as used herein, refers to partially or totally inhibiting, eliminating, delaying, reversing, reducing, or preventing the progression of cancer, including cancer metastasis or malignancy, and/or the recurrence of cancer, including cancer metastasis or malignancy; or preventing the onset or development of cancer in a mammal, in particular, a human. Treating a cancer can involve inhibiting the full development of a tumor or neoplasm, such as by preventing the development of metastasis or by lessening tumor burden.

Treatment of a subject in need thereof typically involves the use or administration of an effective amount or a therapeutically effective amount of an agent, drug, or compound, i.e., an MDM2i according to the invention. Effective amount refers to the quantity (amount) of an agent that induces a desired response in a subject upon administration or delivery to the subject. Optimally, an effective amount produces a therapeutic effect in the absence of, or with minimal to no, adverse effects or cytoxicity in the subject, or wherein the adverse effects are outweighed by the therapeutic benefit achieved. A desired response to an effective amount of an administered agent may be, for example, a decrease in the size, number, or volume of (a) tumor(s) by a desired or significant amount, e.g., by at least 5%, at least 10%, at least 15%, at least 20%, at least 25%, at least 30%, at least 40%, at least 50%, at least 70%, at least 75%, at least 90%, at least 95%, or more, or 100%, compared to a response in the absence of the agent. Alternatively, a desired response may be, for example, an increase in survival time or time of progression-free survival, also by the aforementioned desired or significant percentage amounts.

Regarding “treatment outcome,” the methods of the invention aid in the prediction of an outcome of treatment with an MDM2i. That is, detection of the expression of some (e.g., at least three or at least four), or all, of the genes of the gene signatures described herein in cancer or tumor samples is predictive of an outcome upon treatment with MDM2i. Quantification of an outcome can be an objective response, a clinical response, or a pathological response to treatment with an MDM2i. For example, the outcome can be determined utilizing the techniques for evaluating a response to the treatment of solid tumors as described in Therasse et al., 2000, New Guidelines to Evaluate the Response to Treatment in Solid Tumors, J. Natl. Cancer Inst. 92(3):205-207. Such techniques for determining outcome may involve assessing or measuring survival (including overall survival or the duration of survival), progression-free interval, or survival after recurrence. The timing or duration of these events can be determined from approximately the time of diagnosis, or from approximately the time of initiation of treatment with an MDM2i. Alternatively, outcome can be based upon a reduction in tumor size, tumor volume, or tumor metabolism, or it can be based upon overall tumor burden assessment, or levels of serum markers, particularly in those cases in which such markers are elevated in the disease state (e.g., PSA). Thus, outcome can be characterized as a complete response (CR) to MDM2i, a partial response (PR) to MDM2i, stable disease (SD), and progressive disease (PD), as these terms are conventionally known in the art.

As referred to herein, “sensitivity to treatment” relates to a disease or condition, e.g., a cancer or tumor, that is responsive to an initial, and in some cases, a subsequent or ongoing, therapy or treatment. As an example, a disease or condition that is statistically significantly responsive to an initial, subsequent, or ongoing therapy or treatment is considered to exhibit sensitivity to treatment. Sensitivity may refer to the responsiveness of a disease, symptom, or progression thereof, such as the growth of a cancer or a cancer cell, to an agent or drug, such as a therapeutic agent or drug, for example, an MDM2i, or to a combination of agents, e.g., a combination of one or more MDM2 inhibitors, and/or other anti-cancer drugs. For example, an increased (relative) sensitivity refers to a state in which a cancer is more responsive to a given therapy or therapeutic agent or treatment as compared to a cancer that is not sensitive to the treatment. The term “sensitivity score” as used herein refers to a score obtained from a sample, which reflects the sensitivity of the sample to MDM2i treatment. A sensitivity score can be compared with a threshold to determine whether or not the sample is sensitive to MDM2i treatment. A sensitivity score can be obtained by several statistic methods known to those skill in the art as described below.

The term “threshold”, “cutoff value” and “cutoff point” as used herein refer to a limit with which the sensitivity score of the subject's sample is compared, and are used interchangeably. When the sensitivity score of the subject's sample is over a threshold, the sample can be predicted as sensitive to MDM2i treatment. When the sensitivity score of the subject's sample is under a threshold, the sample can be predicted as resistant to MDM2i treatment. When the sensitivity score of the subject's sample is equal to a threshold, a sample can be predicted as either of sensitive and resistant to MDM2i treatment.

The term “training set” as used herein refers to a set of samples which is used to determine a threshold. The term “test set” as used herein refers to a set of samples which is subjected to a method of the invention to predict whether or not each sample in the set of samples is sensitive to MDM2i treatment. A training set may consist of samples whose sensitivity to MDM2i treatment is known, samples whose sensitivity to MDM2i treatment is unknown, or a combination thereof. A training set can consist of samples whose sensitivity to MDM2i treatment is unknown, alone or in combination with samples whose sensitivity to MDM2i treatment is known, which is described below.

The term “reference score” as used herein refers to a sensitivity score obtained from each sample of a training set, which score can be calculated in the same method as a sensitivity score of the subject is calculated. A threshold can be determined based on the distribution of the reference scores obtained from a training set as described below by statistic methods.

In the field of statistic, the higher a threshold is, the fewer the false positive rate is; and the lower a threshold is, the fewer the false negative rate is. Those skilled in the art would therefore easily and suitably determine a threshold based on the specification. For example, if it is desired to reduce the number of non-responders to MDM2i that is subjected to an MDM2i administration, one can increase a threshold. By increasing a threshold, more responders will be excluded from a group of subjects subjected to MDM2i treatment. If it is desired to increase the number of responders to MDM2i that is subjected to an MDM2i administration, one can decrease a threshold. By decreasing a threshold, more non-responders will be included in a group of subjects subjected to MDM2i treatment.

The term “responder” as used herein refers to a subject that responds to MDM2i treatment. The term “non-responder” as used herein refers to a subject that shows no significant response to MDM2i treatment.

In some cases, sensitivity or responsiveness of a cancer or tumor can be assessed using any parameter or endpoint which indicates a benefit to the subject, including, without limitation (i) an extent of inhibition of cancer, tumor, or neoplasm growth, including growth rate reduction, reduction in progression, and complete growth arrest; (ii) reduction in the number of cancer, tumor, or neoplasm cells; (iii) reduction in cancer, tumor, or neoplasm size or volume; (iv) inhibition, e.g., reduction, lessening, or complete cessation of cancer, tumor, or neoplasm cell infiltration into adjacent peripheral organs and/or tissues; (v) inhibition, e.g., reduction, lessening, or complete cessation, of metastasis; (vi) enhancement of an anti-cancer, tumor, or neoplasm immune response, resulting, optimally, in the regression or rejection of the cancer, tumor, or neoplasm; (vii) relief, to an extent, of one or more symptoms associated with the cancer, tumor, or neoplasm; (viii) increase in the duration of survival/length of survival time following treatment; and/or (ix) decreased mortality subsequent to commencing and/or maintaining treatment.

### DESCRIPTION OF THE EMBODIMENTS

### Gene Signatures Predictive of MDM2i Sensitivity

Provided by the invention is the identification of gene signatures and biomarkers for predicting the sensitivity of cancer and tumors to MDM2 inhibitors as described herein, or to compounds having similar activity. Because expression of genes in the gene signatures is indicative of cancers that are sensitive to MDM2 inhibitors, such gene signatures are also termed “MDM2i gene sensitivity signatures.” The identification of expressed genes in the gene sensitivity signatures in a cancer or tumor sample or specimen from a subject is predictive that the subject, i.e., the subject's cancer or tumor, is sensitive to MDM2i exposure, treatment, or therapy. In addition, the identification of expressed genes in the gene sensitivity signatures can be used to determine and decide upon a therapeutically effective amount of MDM2i or MDM2i treatment regimen to use to treat the subject's cancer or tumor.

In an embodiment, the detection or measurement of gene expression of genes in the gene signatures of the invention in a cancer or tumor sample from a subject undergoing testing indicates a likely beneficial or positive treatment outcome or prognosis for the subject's response or sensitivity to therapy with an MDM2i. In an embodiment, the gene signatures include genes whose expression correlates with a pharmacodynamic effect of an MDM2i therapeutic agent on the MDM2-p53 interaction, or on related signaling pathways, in a subject having a cancer or tumor.

The gene signatures of the invention were derived in a preclinical application by the identification of genes that were differentially expressed in a panel of multi-cancer cell lines sensitive to the small molecule MDM2i, Compound A, as defined herein; i.e., the cell lines exhibited an IC₅₀ for the small molecule inhibitor below a certain threshold or p-value, as compared to cancer cell lines that were not sensitive to the small molecule MDM2i. In an example, the differential expression analysis results permitted a ranking of the genes by p-value according to Student's two class t-Test. The cell line gene signature data were then related to clinical applications, e.g., tissue and cell samples from patients and individuals with cancer, through the identification of a core set of sensitivity signature genes that met pre-specified expression, variance and correlation thresholds in clinical datasets. (See, e.g., Examples 1 and 2). In addition, genes were selected that were elevated in tumors having wild-type TP53 in preclinical and clinical systems and that had increased expression in cancer cells and tissues relative to normal, non-cancerous cells and tissues.

More specifically, 177 genes were identified from the data obtained from a multi-cancer cell line panel (FIGS. 1A-1E); 164 of these genes were selected after excluding those genes encoded by the sex chromosomes. The number of genes was further reduced to 139 based on their correlation with the original 177 gene signature and variable expression in cancer types of interest (7 tumor types) according to the U133 Based Expression Reference containing >28,000 clinical specimens (Compendia Bioscience, Inc., Ann Arbor, Mich.). Of these 139 genes, 38, as presented in Table 3, were selected based on their dependence on TP53 for expression. Thirty seven of these 38 genes (i.e., the genes presented in Table 3, except for PEBP1) showed up-regulated expression in cancer relative to normal tissues. Three genes that are downstream effectors of p53, namely, CDKN1A, SENSN 1 and XPC, were added to this set of 37 genes, which constitutes the final core gene set of 40 genes presented in Table 1.

In accordance with the invention, it was found that at least three genes within the gene signature of FIGS. 1A-1E, Table 1, or the gene set containing the RPS27L, FDXR, CDKN1A and AEN genes can be predictive biomarkers of a cancer or tumor sample's sensitivity to an MDM2i. Preferably, at least three genes of the gene set containing RPS27L, FDXR, CDKN1A and AEN can be predictive biomarkers of a cancer or tumor sample's sensitivity to an MDM2i.

Provided by an embodiment of the invention is a gene signature containing the 177 gene biomarkers as presented in FIGS. 1A-1E in which the differential expression, (generally increased expression), of genes therein in a cancer or tumor sample is predictive of MDM2i sensitivity of that cancer or tumor sample. The differential expression of some or all of the gene components of this gene signature is predictive and indicative of the sensitivity of the cancer or tumor sample to an MDM2i. In some embodiments, the expression of at least 3, at least 4, or all, of the genes within the gene signature of FIGS. 1A-1E is predictive of a cancer or tumor sample's sensitivity to an MDM2i. In some embodiments, expression of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 10, 101, 102, 103, 104, 105, 106, 107, 108, 109, 110, 111, 112, 113, 114, 115, 116, 117, 118, 119, 20, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, or 177 of the genes within the gene signature of FIGS. 1A-1E is predictive of a cancer or tumor sample's sensitivity to an MDM2i and are assayed in the gene signature expression analyses of the invention. In an embodiment, some or all of the genes of this gene signature have increased expressed in the cancer or tumor sample compared with a control. In embodiments, the MDM2i is Compound A and salts thereof, or Compound B and salts thereof, as defined herein. The gene signature of FIGS. 1A-1E comprises those genes that show differential (e.g., increased or elevated) expression in cancer or tumor cells that are sensitive to MDM2i treatment relative to a control.

In some embodiments, the expression, e.g., increased expression, of at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, at least thirty-five, at least thirty-six, at least thirty-seven, at least thirty-eight, at least thirty-nine, or at least forty of the genes within the gene signature presented in Table 1 in a cancer or tumor, or in a sample from a cancer or tumor, relative to a control or a standard value is predictive of sensitivity of an MDM2i. In an embodiment, the standard value is generated from assays with cell lines having known sensitivity or resistance to MDM2 inhibitors. In an embodiment, the MDM2i sensitivity relates to treatment with Compound A and salts thereof or with Compound B and salts thereof as defined herein.

TABLE 1

| Gene | |
|---|---|
| Symbol | p-Value |
| RPS27L | 0.00E+00 |
| FDXR | 0.00E+00 |
| CDKN1A | 0.00E+00 |
| AEN | 1.25E−14 |
| SESN1 | 1.22E−12 |
| TRIAP1 | 1.29E−12 |
| DDB2 | 1.91E−12 |
| XPC | 3.41E−10 |
| C12orf5 | 1.46E−09 |
| BAX | 2.67E−09 |
| PHLDA3 | 3.15E−08 |
| ZMAT3 | 5.34E−08 |
| MDM2 | 4.54E−07 |
| C1QBP | 1.12E−06 |
| SPAG7 | 1.75E−06 |
| TNFRSF10B | 3.79E−06 |
| SLC25A11 | 1.11E−05 |
| SPCS1 | 1.63E−05 |
| GRSF1 | 2.27E−05 |
| GAMT | 2.43E−05 |
| RCBTB1 | 4.33E−05 |
| GDF15 | 4.63E−05 |
| C19orf60 | 5.94E−05 |
| STX8 | 1.21E−04 |
| MED31 | 1.33E−04 |
| POLH | 1.36E−04 |
| GREB1 | 1.91E−04 |
| ACADSB | 2.84E−04 |
| PDE12 | 3.49E−04 |
| EIF2D | 3.53E−04 |
| TIMM22 | 3.64E−04 |
| FAS | 4.76E−04 |
| TP53 | 4.83E−04 |
| HHAT | 5.46E−04 |
| TSFM | 5.55E−04 |
| MPDU1 | 5.62E−04 |
| ISCU | 5.79E−04 |
| METRN | 5.89E−04 |
| DISC1 | 1.07E−03 |
| PRPF8 | 1.17E−03 |

The invention further encompasses a gene signature which comprises or consists of at least three, or all, of the following genes RPS27L, FDXR, CDKN1A and AEN whose expression in a cancer or tumor sample indicates sensitivity of the cancer or tumor to MDM2i. In an example, the MDM2i is Compound A and salts thereof or Compound B and salts thereof as defined herein. In other embodiments, the MDM2i is selected from a spirooxindole derivative, an indole derivative, a pyrrolidine-2-carboxamide derivative, a pyrrolidinone derivative, an isoindolinone derivative, or an imidazothiazole derivative. In other embodiments, the MDM2i is one or more of CGM097, RG7388, MK-8242 (SCH900242), MI-219, MI-319, MI-773, MI-888, Nutlin-3a, RG7112 (R05045337), TDP521252, TDP665759, PXN727, or PXN822.

Expression of the genes within the genes signatures of the invention can be detected using any suitable means known to those skilled in the art. By way of example, the detection of gene expression may be carried out by performing array analysis, including microarrays, as well as by using RT-PCR. Additional methods of detecting gene expression are known in the art and are described in detail below. Differential expression (such as an increase or decrease in expression) of genes in the MDM2i sensitivity gene signatures can be any measurable increase or decrease that is correlated with a sensitivity to MDM2i and/or MDM2i treatment compared with a control.

The invention further provides gene signatures indicating sensitivity of a cancer or tumor sample to MDM2 inhibitors as a companion diagnostic, which can be used by the medical practitioner or clinician who is overseeing the cancer treatment, therapy regimen, or program of an individual with cancer. Companion diagnostics can assist physicians and clinicians (e.g., oncologists) and medical care workers in making treatment decisions for their patients based on the best response to therapy, in particular, to therapy comprising an MDM2i. Companion diagnostics can also assist in the drug development process and lead to more rapid commercialization of drug candidates that are safer, more cost-effective and have better therapeutic efficacy for those who will benefit from a particular type, form, or class of drug. Use of the gene signatures of the invention can assist in the determination of whether a treatment course involving administration of an MDM2i is likely to benefit the individual in terms of reducing, diminishing, abating, eliminating, abrogating, or otherwise affecting the size, growth, proliferation, presence, etc. of a cancer or tumor in ways that are beneficial to the individual. Thus, a companion diagnostic may be beneficial and advantageous if utilized together with the determination of treatment of various types of cancers and tumors with an MDM2i.

Methods Involving Uses of the MDM2i Sensitivity Gene Signatures

The invention generally provides methods for identifying, determining, or predicting if an individual afflicted with a cancer or tumor will be sensitive to treatment with an MDM2i such that treatment with the MDM2i will result in a positive outcome. The methods involve the assessment of whether a biological sample of the individual, e.g., a cancer or tumor tissue or cell sample, differentially expresses genes of the gene sensitivity signatures as described herein, which are indicative of sensitivity to an MDM2i, compared with a control. A positive outcome can encompass one or more of reduction, diminution, elimination, or remission of cancer or cancer cells, as well as apoptosis or death of the cancer cells. A lower, reduced, or lessened tumor burden is also indicative of a positive outcome of MDM2i treatment.

In an embodiment, the invention also provides a method of treating an individual having a cancer or tumor with an MDM2i after determining that the individual is likely to have a positive outcome as a result of the MDM2i treatment. The method involves administering to the individual one or more MDM2 inhibitors in a therapeutically effective amount, as well as for an effective duration, to treat the cancer or tumor. Correlated with the positive outcome of MDM2i treatment is an initial determination of differential expression of genes in the MDM2i gene sensitivity signatures of the invention in a sample obtained from the individual with the cancer or tumor, and then treating the individual with an MDM2i.

In an embodiment, the invention also provides a method of prognosing whether an individual having a cancer or tumor will benefit by, or respond favorably to, treatment with an MDM2i by measuring the expression of genes in the MDM2i gene sensitivity signatures of the invention in a sample obtained from the individual with the cancer or tumor. Accordingly, the sensitivity of a cancer or tumor to MDM2i treatment is assessed before treatment with an MDM2i commences to determine if the cancer or tumor (or individual harboring such pathologies) will likely respond to the treatment. Determining that genes of the gene signatures are expressed in the individual's cancer or tumor sample can involve, for example, determining that the gene expression in the sample has a sensitivity score or rating calculated from the gene sensitivity signature that is above a pre-specified threshold, and is indicative and predictive that the cancer or tumor is sensitive to MDM2i therapy. In some embodiments of the method, the MDM2i administered to the individual undergoing treatment is one or more of the compounds as defined herein. In specific embodiments, the MDM2i is Compound A and salts thereof, or Compound B and salts thereof.

In an embodiment, the use of a sensitivity score can be advantageous, as the score can be used as the basis for defining whether a cancer or tumor is sensitive to an MDM2i and can thus be predictive that an individual having an MDM2i sensitive cancer or tumor will respond favorably to MDM2i treatment. For example, upon the determination of a sensitivity score indicative of a cancer or tumor sample's sensitivity to an MDM2i, a medical practitioner may elect to treat a patient having the cancer or tumor with an MDM2i drug or compound. Alternatively, upon the determination of a sensitivity score indicative of a cancer or tumor sample's insensitivity to an MDM2i, a medical practitioner may elect not to treat a patient having the cancer or tumor with an MDM2i drug or compound, as the patient would be predicted not to receive a clinical or medical benefit from MDM2i treatment. If a sample of a patient's cancer or tumor is assessed for MDM2i sensitivity during a course of cancer drug treatment or therapy, a sensitivity score indicative of MDM2i sensitivity may assist the medical practitioner in deciding to continue or alter the patient's cancer or tumor treatment or therapy and/or to treat with an MDM2i.

According to the invention, a sensitivity score of each sample and a threshold for predicting the sensitivity of a subject can also be obtained from samples whose sensitivities to MDM2i treatment are unknown. This is one of the very important features of the invention because the sensitivity of the subject to MDM2i treatment can be predicted in the invention even when the sensitivities to MDM2i treatment in a specific group of samples are totally or almost unknown. For example, none of human clinical trials' data on MDM2i sensitivity has been available. Thus, nobody can tell who is resistant to MDM2i treatment or who should not be treated by MDM2i treatment. However, in an aspect, the invention provides a method for predicting sensitivity to MDM2i treatment of a subject (preferably a human subject) using samples whose sensitivities to MDM2i treatment are unknown.

In an embodiment, a threshold is determined from the distribution of sensitivity scores of samples wherein sensitivities of at least a part or all of the samples to MDM2i treatment are unknown. In particular, a sensitivity score and a threshold can be obtained by using statistical methods such as score extrapolation models, Gaussian mixture models and other models known to those skilled in the art. In this embodiment, more than 50%, 60%, 70%, 80%, 90% or 100% of the samples from which a threshold is determined may be samples whose sensitivities to MDM2i treatment are unknown.

In score extrapolation models, a sensitivity score of a sample can be calculated by summing the normalized score (also referred to as “standard score”, “z-value”, “z-score”, “normal score”, or “standardized variable”) of the expression level of each gene in the sample, which can be calculated from the following function: (normalized score)={(raw value of gene expression)−(average of the distribution)}/(standard deviation of the distribution). According to the invention, a threshold can also be determined from the samples whose sensitivities to MDM2i are unknown, by plotting a Receiver Operating Characteristic (ROC) curve and optionally conducting leave-one-out cross-validation (LOOCV) analysis. In an embodiment, a threshold can be within the Youden idex±0.3, ±0.2, ±0.1 or the Youden index of the ROC curve, and may range between −0.2 and 0.5, preferably between −0.02 and 0.2, and more preferably be about −0.02, about 0.14 or about 0.2. In another embodiment, a threshold can be the cut off value of the point on the ROC curve which is located closest to where true positive rate is 1 and false negative rate is 0. In an embodiment, a threshold can be determined by binalization algorithms such as Otsu's method, which is a well-known clustering-based image thresholding method and also known as “Otsu's thresholding” (see M. Sezgin and B. Sankur (2004), Journal of Electronic Imaging 13 (1): 146-165, and N. Otsu (1979), IEEE Trans. Sys., Man., Cyber 9 (1): 62-66). In an embodiment, in step d), a threshold ranges between the values of the third quartile and the maximum of the reference scores of TP53 mutant samples among the samples; or between the values of the first quartile and the minimum of the reference scores of TP53 wild type samples among the samples. In an embodiment, in step d), a threshold can be determined as the level of the third quartile or the maximum of the sensitivity scores of TP53 mutant samples among the samples. In another embodiment, a threshold can alternatively be determined as the level of the first quartile or the minimum of the sensitivity scores of TP53 wild type samples among the samples. A sample is predicted as sensitive when the sensitivity score of the sample is higher than the threshold, and is predicted as resistant when the sensitivity score is lower than the threshold. The terms “first quartile” and “third quartile” as used herein mean the bottom 25^{th} percentile and the top 25^{th} percentile, respectively.

In Gaussian mixture models, a sensitivity score can be calculated by first determining the two Gaussian distributions as follows. In order to create Gaussian mixture models, a commercially available “mclust” package (ver. 4.3), which was developed by C. Fraley et. al. (Technical Report no. 597, Department of Statistics, University of Washington, June 2012) can be used on R statistics software (ver. 3.0.2). In particular, Gaussian mixture models can be created as follows. In a cell line panel that consists of sensitive and resistant cell lines, the distribution of mRNA expression of a signature gene can be described as a mixture of the distribution derived from sensitive cell lines and resistant cell lines. If the distributions are supposed to be normal distributions, the mixed distributions are described as the Gaussian mixture model:

[Math. 1]

p(x|λ)=Σᵢ₌₁²ωᵢg(x|μᵢ,σ). (1)

λ is a set of parameters: λ={ωᵢ, μᵢ, σ}, i=1, 2 and ωᵢ, i=1, 2 are the mixture weights, and g(x|μᵢ, σ), i=1, 2 are the component Gaussian densities. Each component density is a Gaussian function of the form,

$$\begin{matrix}[ Math . 2 ] & \\ g ( x \vert {μ}_{i} , σ ) = \frac{1}{\sqrt{2 {πσ}^{2}}} exp ( - \frac{{( x - {μ}_{i} )}^{2}}{2 {σ}^{2}} ) , i = 1 , 2 , & ( 2 )\end{matrix}$$

with the mean μᵢ, i=1, 2. For convenience, μᵢ satisfy the constraint that μ₁<μ₂. The standard deviation σ is supposed to be common between sensitive and resistant cell lines, i.e., σ=σ₁=σ₂. Each parameter can be estimated by maximum likelihood estimation, which is to find model parameters maximizing the likelihood of the model given the training data. For a sequence of T training vectors X={x₁, . . . , x_{T}}, the likelihood can be written as,

[Math. 3]

p(X|λ)=Π_{z=1}^{T}p(xₜ|λ). (3)

Maximum likelihood parameters can be obtained by Expectation-Maximization (EM) algorithm. The basic idea of the EM algorithm is to estimate a new parameter \hat{λ} from the previous parameter λ such that p(X|\hat{λ})≧p(X|λ). The λ is repeatedly renewed until the likelihood converges on a maximum value.

In the Gaussian mixture models, thresholds can be obtained by several methods. The number of the genes whose expressions indicate that the cell is sensitive can be useful in determining a threshold. For M genes, when the expression of each gene is written as xₘ, m=1, . . . , M, the class of gene m can be written as,

$$\begin{matrix}[ Math . 4 ] & \\ {C}_{m} = {argmax}_{i} \{ {ω}_{i} g ( {x}_{m} \vert {μ}_{i} , σ ) \} , i = 1 , 2 , m = 1 , … , M . & ( 4 )\end{matrix}$$

where Cₘ is 1 or 2, and we call the gene is ‘lower’ when Cₘ=1 and ‘upper’ when Cₘ=2. When a variable,

$$\begin{matrix}[ Math . 5 ] & \\ {u}_{m}^{{c}_{m}} = \{ \begin{matrix}0 , & {C}_{m} = 1 \\ 1 , & {C}_{m} = 2\end{matrix} , & ( 5 )\end{matrix}$$

is introduced to describe the class to which the gene m belongs, the ‘upper ratio’ is given by

$$\begin{matrix}[ Math . 6 ] & \\ ( upper ratio ) = \frac{{\sum }_{m = 1}^{M} {u}_{m}^{{C}_{m}}}{M} . & ( 6 )\end{matrix}$$

In an embodiment, threshold of a sensitivity score to MDM2 inhibitor can be determined by referring to the upper ratio (6). For example, a threshold can be determined as the level of the third quartile or the maximum of the upper ratios of TP53 mutant samples selected from the samples. A threshold can alternatively be determined as the level of the first quartile or the minimum of the upper ratios of TP53 wild samples selected from the samples. A sample is predicted as sensitive when the upper ratio of the sample is higher than the threshold, and is predicted as resistant when the upper ratio is lower than the threshold.

In score distribution models, a threshold can be obtained based on the shape of the score distribution histogram for each gene. In an embodiment, a threshold can be determined based on the value of the score where the score distribution forms a valley (i.e, the lowest part of a concave portion of the histogram), preferably by Otsu's method. In an embodiment, the score distribution histogram can be obtained by summing z-scores calculated from the expression levels of the genes analyzed. A sample is predicted as sensitive when the score of the sample is higher than the threshold, and is predicted as resistant when the score is lower than the threshold. In a score distribution model, a threshold can alternatively be determined from the score distribution histogram where all of the scores have been obtained from TP53 wild type samples, and preferably be determined as the first quartile value of the scores.

In an embodiment, a threshold can also be determined by using the following formula (7).

$$\begin{matrix}[ Math . 7 ] & \\ ( Likelihood \cdot ratio ) = {\prod }_{m = 1}^{M} \frac{{ω}_{2} g ( {x}_{m} \vert {μ}_{2} , σ )}{{ω}_{1} g ( {x}_{m} \vert {μ}_{1} , σ )} & ( 7 )\end{matrix}$$

Sensitivity to MDM2 inhibitor of a sample of interest can be determined by referring to the likelihood ratio (7) of the sample, for example, as sensitive if the ratio is over a threshold and as resistant if the ratio under the threshold, wherein the threshold ranges between 0.2 and 5, preferably between 0.5 and 2, more preferably between 0.8 and 1.25 and still more preferably about 1.

In other embodiments, the invention provides methods, reagents and information conducive to improving treatments and treatment options for individuals afflicted with cancer, wherein the individuals can benefit from treatment or therapy with an MDM2i drug, agent, or compound. As will be appreciated by the skilled practitioner, the MDM2 inhibitors pursuant to the invention are preferably used and administered to a subject in a therapeutically effective amount, which is intended to qualify as the amount or dose of the treatment, such as a drug, compound, active ingredient, composition, or agent, determined or necessary to treat cancer in a therapeutic or treatment regimen. This includes combination therapy involving the use of multiple MDM2 inhibitors, or multiple therapeutic agents, such as a combined amount of a first and second treatment, in which the combined amount will achieve the desired biological treatment response.

In accordance with the invention, the MDM2i can be administered by any route conventionally used for drug administration and as known to the skilled practitioner. By way of non-limiting example, an MDM2i can be administered orally, parenterally, intravenously, subcutaneoulsy, bucally, sublabially, intranasally, intradermally, sublingually, intrathecally, intramuscularly, intraperitoneally, rectally, intravaginally, gastrically, or enterically. Oral administration, e.g., in tablet, capsule, or liquid form, is preferred. An MDM2i can be administered as a single dose, or in multiple doses, as needed, to obtain a desired response. As will be appreciated by the skilled practitioner, the dose for administration will depend upon the individual undergoing treatment, the severity and type of the condition being treated and the manner of administration.

The methods of the invention can be used to determine the sensitivity or responsiveness of a cancer or tumor to a therapy, in particular, MDM2i or antagonist therapy, or to determine the prognosis of a subject with a cancer or neoplasm. In this way, the invention provides methods of treating patients suffering from cancer wherein the cancer is sensitive to an MDM2i based upon the detection of the expression of genes in the gene sensitivity signatures in the cancer tissue (or by a sensitivity score or rating obtained by analysis of the gene signature).

In an embodiment, the invention provides a method of predicting sensitivity of a subject having a cancer, tumor, or neoplasm to treatment with an MDM2i, in which the method involves detecting the differential expression of a plurality of genes in an MDM2i sensitive gene signature of the invention in a cancer, tumor, or neoplasm sample obtained from the subject, wherein the plurality of genes comprises, or consists of, at least three, at least four, or all, of the genes set forth in FIGS. 1A-1E; or in the gene signature having the genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC; or in the gene signature having the genes MDM2, CDKN1A, ZMAT3, DDB2, FDXR, RPS27L, BAX, RRM2B, SESN1, CCNG1, XPC, TNFRSF10B and AEN; or in the gene signature having the genes RPS27L, FDXR, CDKN1A, and AEN (and optionally MDM2); and comparing the expression of the gene signature genes in the cancer, tumor, or neoplasm sample to a control. In the method, an increase in expression of at least three, at least four, or all, of the genes set forth in FIGS. 1A-1E; or in the gene signature having the genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC; or in the gene signature having the genes MDM2, CDKN1A, ZMAT3, DDB2, FDXR, RPS27L, BAX, RRM2B, SESN1, CCNG1, XPC, TNFRSF10B and AEN; or in the gene signature having the genes RPS27L, FDXR, CDKN1A, and AEN (and optionally MDM2) in the cancer, tumor, or neoplasm sample relative to the control indicates sensitivity of the cancer, tumor, or neoplasm to the MDM2i, thereby predicting the sensitivity of the subject to the MDM2i treatment. By way of example, some embodiments include detecting a difference in the expression levels of three or more, four or more, five or more, six or more, or all, (such as at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or at least 40) of the MDM2i sensitivity signature genes in a cancer or tumor sample obtained from a subject with the cancer or tumor compared to a control, such as a reference value, or a non-cancer, tumor, or neoplasm tissue sample from a healthy subject, or from a non-tumored tissue sample from the subject undergoing testing. In an embodiment of the method, the increase in expression relative to control can be, without limitation, an increase of at least about 1.5-fold, at least about 2-fold, at least about 2.5-fold, at least about 3-fold, at least about 3.5-fold, at least about 4-fold, at least about 5-fold, at least about 6-fold, at least about 8-fold, or at least about 10-fold.

The invention further provides methods of determining whether an individual's cancer or tumor is, or is likely to be, sensitive to treatment with an MDM2i, as well utilizing this determination to treat the individual whose cancer is sensitive to the inhibitor with an MDM2i. Also provided are methods of predicting or prognosing whether an individual with cancer is likely to respond to or benefit from treatment with an MDM2i. According to an embodiment, in a method of the invention, a sample of a patient's cancer or tumor, in the form of archived samples or fresh biopsies, for example, is analyzed prior to MDM2i therapy for the expression levels of genes within an MDM2i sensitivity gene signature as described herein, relative to a control wherein the composite expression level of the genes in the gene signature can be reported as a sensitivity score. As will be appreciated by the skilled practitioner, deriving a sensitivity score from actual tumor samples collected from patients, (for example, subjects in clinical studies with an MDM2i drug), may differ from deriving such a score from preclinical studies. By way of example, the sensitivity score derived for tumor samples from clinical samples could utilize the expression levels of one or more constitutively expressed genes, while in samples from preclinical studies, the score may be derived relative to gene levels of other samples in a population. Nonetheless, it is expected that tumor samples with a sensitivity score above a certain cutoff value will have a higher likelihood of responding to the MDM2i. The cutoff value may be further determined based on validation studies using clinical samples with known TP53 genotyping status, as well characterized p53 mutants are expected to show a low sensitivity score. In addition, the cutoff value can also be adjusted upon correlation of tumor response during clinical trials involving treatment with an MDM2i, for example, Compound B and salts thereof.

In an embodiment, the invention provides a method of identifying whether a cancer or tumor is sensitive to treatment with an MDM2i, or predicting the sensitivity of a cancer or tumor to an MDM2i, by detecting differential expression levels (e.g., increased expression levels compared with a control) of at least three, at least four, at least five, at least six, or all, of the genes in the gene signature listed in FIGS. 1A-1E, or in gene set including BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and/or XPC, or in gene set including MDM2, CDKN1A, ZMAT3, DDB2, FDXR, RPS27L, BAX, RRM2B, SESN1, CCNG1, XPC, TNFRSF10B and AEN; or in the gene set including RPS27L, FDXR, CDKN1A, and AEN (and optionally MDM2) in a cancer or tumor sample obtained from a subject, and identifying the cancer or tumor as sensitive to treatment with an MDM2i if there is a difference in the level of expression of some or all of the genes in the cancer sample as compared to a control, or based on a sensitivity score or rating generated from the gene sensitivity signature of the sample that is above a determined threshold or cutoff value and is thus indicative of sensitivity of the sample to the MDM2i. Detecting and measuring the levels of expression of the genes indicating MDM2i sensitivity in the cancer or tumor sample from the subject can be performed by a method known in the art and as described herein. In various embodiments, the MDM2i is Compound A and salts thereof, Compound B and salts thereof, a spirooxindole derivative, an indole derivative, a pyrrolidine-2-carboxamide derivative, a pyrrolidinone derivative, an isoindolinone derivative, or an imidazothiazole derivative, and salts thereof. In some embodiments, the MDM2i is CGM097, RG7388, MK-8242 (SCH900242), MI-219, MI-319, MI-773, MI-888, Nutlin-3a, RG7112 (R05045337), TDP521252, TDP665759, PXN727 or PXN822, and salts thereof.

In some embodiments, the invention provides methods for determining a pharmacodynamic effect of MDM2i treatment or therapy on a cancer or tumor sample, involving detecting a difference in the levels of expression of three or more, four or more, five or more (such as at least six), or all, of the gene signature biomarker genes listed in FIGS. 1A-1E; in Table 1; or in the gene signature containing genes RPS27L, FDXR, CDKN1A and AEN (and optionally MDM2) in the cancer or neoplasm sample relative to a control. By way of example, some embodiments include detecting a difference in the expression levels of three or more, four or more, five or more, six or more, or all, (such as at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or at least 40) of the MDM2i sensitivity signature genes in a cancer or tumor sample obtained from a subject with the cancer or tumor compared to a control, such as a control cancer or tumor sample obtained from the subject before therapy was initiated, or other appropriate control, wherein the genes comprise the gene signatures as described herein, such as those as set forth in FIGS. 1A-1E and in Table 1 herein.

In an embodiment of the methods, the cancer or tumor sample undergoing analysis can also be assayed to determine if it has a wild-type TP53 gene by methods known in the art. In an embodiment, a wild type TP53 gene can be associated with a tumor cell's sensitivity to inhibitors or antagonists of the p53-MDM2 protein-protein interaction; however, some diversity in response to such agents may be observed among TP53 wild type cancer cell types and tumor models. In an embodiment, the invention provides a method for predicting sensitivity of an individual's cancer or tumor to treatment with an MDM2i by measuring the levels of expression of at least three, or at least four genes selected from the genes in the gene signatures of the invention, e.g., in FIGS. 1A-1E; in Table 1; or in the gene set RPS27L, FDXR, CDKN1A, and AEN (and optionally MDM2), in a cancer or tumor sample obtained from the individual and determining if the cancer or tumor sample has a wild-type TP53 gene.

In another embodiment, the invention provides a method of predicting the sensitivity of a subject's cancer or tumor to MDM2 inhibitor treatment, in which the method involves a) measuring the levels of expression of at least three or at least four genes selected from the genes listed in FIGS. 1A-1E in a cancer or tumor sample obtained from the subject and b) determining if the cancer or tumor sample has a wild-type TP53 gene. In another embodiment, the invention provides a method of treating a subject having a cancer or tumor, in which the method comprises: a) assessing the sensitivity of a subject's cancer or tumor to MDM2 inhibitor treatment, comprising measuring the levels of expression of at least three or at least four genes selected from the genes listed in FIGS. 1A-1E in a cancer or tumor sample obtained from the subject; b) determining if the cancer or tumor has a wild-type TP53 gene; and c) administering to the subject an effective amount of an MDM2 inhibitor to treat the cancer or tumor, if the assessment of step a) indicates that the cancer or tumor is sensitive to the MDM2 inhibitor and the cancer or tumor specimen has a wild-type TP53 gene.

An advantage afforded by the invention is that the described methods of assessing the expression levels of genes in the disclosed gene signatures outperform TP53 genotyping alone in predicting the sensitivity of a cancer or tumor sample to MDM2 inhibitors. Thus, the described methods provide a benefit and improvement in the art for the determination of treatment for cancers and tumors with an MDM2i. An illustrative, yet nonlimiting, example of such a case occurs with respect to cervical cancers, which by and large are infected with the human papilloma virus (HPV) which produces the E6 oncoprotein that down-regulates p53 function. While cervical cancer cells are often found to be wild type for TP53, they are typically insensitive to MDM2 inhibition. For example, as shown in Table 2, TP53 wild type C4II, C4I, SiHa and, Hela that were infected with HPV (HPV18:C4II and C4I; HPV16:SiHa and Hela) were insensitive to MDM2i, and showed low expression levels of the genes within MDM2i gene signatures of the invention, a result that likely relates to their infection by HPV and its associated intracellular effects. Accordingly, the MDM2i gene sensitivity signatures of the invention and methods involving their use may be the sole means, or the most reliable means, for more accurately predicting whether a given cancer or tumor type is likely to be sensitive to treatment with an MDM2i. As such, the invention provides both time and cost saving benefits for patient treatment and personalized medical care.

In an aspect, the invention provides a method for predicting the sensitivity of a subject's cancer or tumor to MDM2i treatment, comprising: a) measuring the levels of expression of at least three genes selected from the genes listed in FIGS. 1A-1E in a cancer or tumor sample obtained from the subject; b) scoring the levels of expression obtained in step a) to obtain a subject's sensitivity score; c) measuring the levels of expression of the at least three genes in plurality of cancer or tumor samples, wherein sensitivities to MDM2i treatment of at least a part of the samples are unknown; d) scoring the levels of expression obtained in step c) to obtain a reference score in each sample and determining a threshold based on the distribution of the reference scores; and e) predicting that the subject is sensitive to MDM2i treatment if the subject's sensitivity score is over the threshold and the subject is resistant to MDM2i treatment if the subject's sensitivity score is under the threshold. In an embodiment, the number of cancer or tumor samples to be measured in step c) may be 4 or more, 6 or more, 8 or more, 10 or more, 15 or more, 20 or more, 30 or more, 40 or more, 50 or more, 100 or more, 200 or more, 300 or more, 400 or more, 500 or more, or 1000 or more. In a specific embodiment, the number of cancer or tumor samples to be measured in step c) may be between 6 and 20. In an embodiment, step d) may comprise grouping the samples into a group of TP53 wild type samples and a group of TP53 mutant samples, and then determining a threshold based on the distributions of the reference scores of TP53 wild type samples and/or TP53 mutant samples. In a particular embodiment, the threshold in e) in the method of the invention ranges between the values of the third quartile and the maximum of the reference scores of TP53 mutant samples among the samples. In another embodiment, the threshold in e) ranges between the values of the first quartile and the minimum of the reference scores of TP53 wild type samples among the samples.

In a particular embodiment, the method further comprises step f) predicting that the subject is sensitive to MDM2i treatment if the subject that is predicted as resistant shows an MDM2 overexpression. The Inventors discovers that tumors which are sensitive to MDM2i treatment are sometimes predicted as resistant according to steps a) to d) in the invention, and that this wrong prediction is caused by overexpression of MDM2 in the tumors. The Inventors further discovers that the wrong prediction can be correctly modified by determining MDM2 overexpression in the tumors. Therefore, in an embodiment, the invention provides a method for predicting the sensitivity of a subject's cancer or tumor to MDM2i treatment, comprising: a) measuring the levels of expression of at least three genes selected from the genes listed in FIGS. 1A-1E in a cancer or tumor sample obtained from the subject; b) scoring the levels of expression obtained in step a) to obtain a subject's sensitivity score; c) measuring the levels of expression of the at least three genes in plurality of cancer or tumor samples, wherein sensitivities to MDM2i treatment of at least a part of the samples are unknown; d) scoring the levels of expression obtained in step c) to obtain a reference score in each sample and determining a threshold based on the distribution of the reference scores; e) predicting that the subject is sensitive to MDM2i treatment if the subject's sensitivity score is over the threshold and the subject is resistant to MDM2i treatment if the subject's sensitivity score is under the threshold; and f) predicting that the subject is sensitive to MDM2i treatment if the subject that is predicted as resistant shows an MDM2 overexpression. The Inventors further discovers that tumors whose TP53 genes are wild type are sometimes predicted as resistant according to steps a) to d) in the invention, although the tumors are actually sensitive to MDM2i treatment, and that this wrong prediction is caused by overexpression of MDM2 in the tumors. Therefore, in an embodiment, step 0 can be a step of predicting that the subject is sensitive to MDM2i treatment if the subject that is predicted as resistant shows an MDM2 overexpression and has wild type TP53 genes. Those skilled in the art can determine whether or not the subject shows an MDM2 overexpression, comparing the expression level of MDM2 in the subject with the average expression level of MDM2 in the training sets wherein sensitivities to MDM2i treatment of at least a part of the samples are unknown. Those skilled in the art can determine that the subject shows an MDM2 overexpression when the expression level is 5 fold or more, 10 fold or more, 20 fold or more, 50 fold or more, 100 fold or more, 200 fold or more, 500 fold or more, 1000 fold or more, 2000 fold or more, or 3000 or more stronger than the average. In an embodiment, an MDM2 overexpression can be caused by an amplification of MDM2 genes in the genome of the subject.

Alternatively, the invention provides a method for predicting the sensitivity of a subject's cancer or tumor to MDM2i treatment, comprising: x) determining the genotype of TP53 and the level of MDM2 expression in the subject's cancer or tumor, y) predicting the subject as sensitive to the treatment if TP53 is wild type and the cancer or tumor shows an MDM2 overexpression over the average of that in samples whose sensitivity to MDM2i treatment is unknown, performing the following steps unless TP53 is wild type and the level of MDM2 expression is over the average: a) measuring the levels of expression of at least three genes selected from the genes listed in FIGS. 1A-1E in a cancer or tumor sample obtained from the subject; b) scoring the levels of expression obtained in step a) to obtain a subject's sensitivity score; c) measuring the levels of expression of the at least three genes in plurality of cancer or tumor samples, wherein sensitivities to MDM2i treatment of at least a part of the samples are unknown; d) scoring the levels of expression obtained in step c) to obtain a reference score in each sample and determining a threshold based on the distribution of the reference scores; e) predicting that the subject is sensitive to MDM2i treatment if the subject's sensitivity score is over the threshold and the subject is resistant to MDM2i treatment if the subject's sensitivity score is under the threshold.

In an embodiment, steps b) and d) comprise summing the normalized scores of the levels of the gene expression. In an embodiment, the threshold is determined based on Receiver Operating Characteristic (ROC) plots optionally by conducting leave-one-out cross-validation (LOOCV) analysis. In a particular embodiment, the threshold falls within the Youden Index±0.3, preferably +0.2, and more preferably ±0.1 and still more preferably is substantially equal to the Youden Index of the Receiver Operating Characteristic (ROC) curve.

In an embodiment, the threshold is determined from the shape of the reference scores by binalization algorithms such as Otsu's method.

In an embodiment, the threshold is determined by Gaussian Mixture model. In an embodiment, the threshold is determined based on a ratio of the number of the genes which indicates the subject as sensitive to that of the genes which indicates the subject as resistant by using two Gaussian distributions in Gaussian Mixture model in step d). In a particular embodiment, the threshold in step e) ranges between the values of the third quartile and the maximum of the ratios of TP53 mutant samples among the samples; or between the values of the first quartile and the minimum of the ratios of TP53 wild type samples among the samples. In an embodiment, the invention provides a method for predicting the sensitivity of a subject's cancer or tumor to MDM2i treatment, comprising performing a plurality of predictions, wherein each prediction comprises the above-mentioned steps a) to d), steps a) to e), or steps a) to f) and wherein at least a part or all of cancer samples or tumor samples in step c) are different among the predictions, and predicting that the subject is sensitive to MDM2i treatment if the number of the prediction results indicating the subject as sensitive is 50% or more, 60% or more, 70% or more, 80% or more, 90% or more of the total number of the predictions performed and that the subject is resistant to MDM2i treatment if the number of the prediction results indicating the subject as sensitive is under the above mentioned percentage. In a specific embodiment, the method comprising performing 10 or more, 20 or more, 30 or more, 40 or more, 50 or more, 60 or more, 70 or more, 80 or more, 90 or more, or 100 or more predictions each of which comprises the above-mentioned steps a) to d), steps a) to e), or steps a) to f) and wherein at least a part or all of cancer samples or tumor samples in step c) are different among the predictions.

In an aspect, the invention provides a method for treating a subject having a cancer or tumor, comprising: a) assessing the sensitivity of a subject's cancer or tumor to MDM2i treatment by the present method using samples whose sensitivities to MDM2i are unknown as a training set; and b) if the assessment indicates that the cancer or tumor is sensitive to the MDM2i, administering to the subject an effective amount of an MDM2i to treat the cancer or tumor.

In an aspect, the invention provides a pharmaceutical composition for use in treating a cancer or tumor in a subject, wherein the composition comprises an MDM2i, and wherein the subject is determined as sensitive to the MDM2i treatment by assessing the sensitivity of a subject's cancer or tumor to the MDM2i treatment by the present method using samples whose sensitivities to MDM2i are unknown as a training set.

In an embodiment, a cancer or tumor can be melanoma.

In an embodiment, the results of the gene expression/gene signature analysis as afforded by the methods of the invention may be provided to a practitioner or user, such as a clinician or other medical professional or healthcare worker, laboratory personnel, or a patient in a perceivable output that provides information about the results of the analysis. In some instances, the output can be in paper or graphic form, such as a written or printed copy, or a chart, graph, or diagram, or viewable on a display (e.g., a computer screen), or as an audible output. In an embodiment, the output is a numerical value in a sample, or a relative amount of one or more of the gene signature genes in the sample, compared to a control. The numerical value can be, for example, for a gene signature as described herein for MDM2i sensitivity, or for p53 status, or for an expression level of a gene or set of genes, e.g., comprising a gene signature as described herein, compared to a control value. In cases in which the output is in graph form, the graphical representation can be a graph which indicates the value(s), e.g., an amount or relative amount, of a gene or gene set as described in the gene signatures herein, in a sample from a subject plotted against a standard curve. The output, or graphical output, can demonstrate or provide a cutoff value or level that indicates that the cancer is sensitive to the MDM2i. The output can predict that the subject has a cancer or tumor that is more likely to be sensitive to the MDM2i treatment if the value or level is above the cutoff value. The output can be communicated to the user via its being provided or transmitted by electronic, audible or physical means, e.g., by mail, email, facsimile, telephone, or electronic medical record communication. Alternatively, the output can indicate that the subject's cancer or neoplasm is less likely to be sensitive to MDM2i treatment if the value or level is below the cutoff.

In some embodiments, the output is communicated to the user, for example, by providing an output via physical, audible, or electronic means (for example by mail, telephone, facsimile transmission, email, or communication to an electronic medical record). The various types of output can provide quantitative information, for example, the level or amount of a gene or set of genes in a gene signature, which is found in a sample, or an amount or level of a gene or gene set as described relative to a control sample or control value. Such output can also provide qualitative information, for example, a determination of MDM2i sensitivity or a prognosis of MDM2i sensitivity. The output can further provide qualitative information regarding the relative amount(s) of one or more of the genes within a gene signature in a sample, such as identifying or revealing an increase or a decrease in the expression of one or more, at least three, or at least four of the described genes or gene sets relative to a control, or no difference among one or more of the described genes or gene sets relative to a control. In some cases, the gene expression analysis can include a determination of other clinical information, such as a determination of the amount or level of one or more additional cancer biomarkers in the sample. In some cases, the gene expression analysis or test can include an array, such as an oligonucleotide or antibody array, and the output of the analysis or test can include quantitative and/or qualitative information about one or more of the disclosed gene components of the gene signatures of the invention, as well as quantitative and/or qualitative information about one or more additional genes.

### Cancer and Tumor Types and Subtypes

A patient undergoing testing to determine sensitivity of his/her cancer or tumor specimen to an MDM2i may suffer from a cancer or tumor of essentially any tissue or organ, and the cancer or tumor specimen may be obtained from the patient by a procedure prior to the selection or initiation of MDM2i treatment, as described herein. The cancer or tumor may be primary or recurrent, and may be of any type (as described herein), stage (e.g., Stage I, II, III, or IV or an equivalent of other staging system), and/or histology. The patient may be of any age, sex, performance status, and/or extent and duration of disease or remission. A gene expression profile may be determined for a tumor tissue or cell sample, such as a tumor sample that has been removed from a patient by surgery or biopsy. In some cases, the cancer or tumor sample, or cells therefrom, may be established in cell culture or as xenografts in immunocompromised animals. In some cases, the sample may be frozen after removal from the patient, and preserved for later RNA isolation. The sample for RNA isolation may be a formalin-fixed paraffin-embedded (FFPE) tissue. Processes for enriching or expanding malignant cells in culture may be found, for example, in U.S. Pat. Nos. 5,728,541, 6,900,027, 6,887,680 and 6,933,129.

In some embodiments, the cancer or tumor with which a subject is afflicted and/or which is undergoing assessment according to the methods of the invention is a solid tumor or neoplasm, such as a carcinoma or a sarcoma, including, for example, fibrosarcoma, myxosarcoma, liposarcoma, chondrosarcoma, osteosarcoma, mesothelioma, Ewing's tumor, leiomyosarcoma, rhabdomyosarcoma, colon carcinoma, pancreatic cancer, breast cancer, lung cancers, ovarian cancer, prostate cancer, synovioma, squamous cell carcinoma, basal cell carcinoma, sweat gland carcinoma, sebaceous gland carcinoma, papillary carcinoma, papillary adenocarcinoma, medullary carcinoma, bronchogenic carcinoma, renal cell carcinoma, adenocarcinoma, hepatoma, hepatocellular carcinoma, bile duct carcinoma, choriocarcinoma, Wilms' tumor, cervical cancer, testicular tumor, bladder carcinoma, and brain and central nervous system (CNS) tumors, such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

In other embodiments, the tumor or neoplasm includes an abnormal cell growth occurring in a hematological cancer, for example, leukemias, such as leukemias, e.g., acute lymphoblastic leukemia, acute myelocytic leukemia, acute myelogenous leukemia, myeloblastic leukemia, promyelocytic leukemia, myelomonocytic leukemia, monocytic leukemia and erythroleukemia; chronic leukemias, for example, chronic myelocytic (granulocytic) leukemia, chronic myelogenous leukemia, and chronic lymphocytic leukemia; polycythemia vera; lymphoma; lymphoid malignancy, Hodgkin's disease; non-Hodgkin's lymphoma, e.g., indolent and high grade forms; including Burkitt's lymphoma and mantle cell lymphoma; multiple myeloma; plasmacytoma; Waldenstrom's macroglobulinemia; heavy chain disease; myelodysplastic syndrome and myelodysplasia.

In particular embodiments, the cancers or tumors for which MDM2 inhibitors may be used as treatment, and for which the gene signatures and related methods of the invention can be particularly applied, include a variety of solid tumors, including soft tissue tumors, as well as blood cancers. Illustratively, without limitation, the cancer types (or subtypes) for which the gene signatures of the invention can have particular relevance include myeloma, multiple myeloma, melanoma, lymphoma, leukemia (e.g., ALL, AML), kidney, brain and central nervous system (CNS), sarcoma, gastric, cervical, prostate, breast, liver, renal, bladder, lung (e.g., NSCLC), pancreas, head and neck, colorectal, esophageal, testes, prostate, ovary, cervix, and others. In an embodiment, use of the gene signatures indicative of sensitivity to MDM2 inhibitors is particularly beneficial for treating cancer types such as melanoma, myeloma, glioblastoma, lymphoma (e.g., DLBCL), leukemia, brain and CNS cancers, and sarcomas. In an embodiment, the cancer or tumors have functional p53 protein.

In other embodiments, particular cancer subtypes, such as renal Wilm's tumor, granular renal cell carcinoma, renal oncocytoma, Burkitt's lymphoma, monoclonal gammopathy of undetermined significance, papillary renal cell carinoma, melanoma, multiple myeloma, cutaneous myeloma, chromophobe renal cell carcinoma, cutaneous T-cell lymphomas (e.g., Mycosis Fungoides and Sezary Syndrome), oligodendroglioma, astrocytoma, acute myelogenous leukemia, acute lymphoblastic leukemia, gliobastoma, endometrial mixed adenocarcinoma, colorectal adenoma, parathyroid gland adenoma, synovial sarcoma, fibrosarcoma and thyroid gland carcinoma, score highly for MDM2i sensitivity, thereby making them especially relevant for treatment with MDM2 inhibitors and for achieving a likely positive response to MDM2i treatment. These cancer subtypes are likely to exhibit expression of genes in the gene signatures of the invention and to be sensitive to treatment with an MDM2i. In a particular embodiment, nonlimiting examples of cancer types and subtypes included among those that are determined to have a high frequency of sensitivity to MDM2 inhibitors, such as Compounds A and B described herein, are renal tumors (e.g., Wilm's tumor), lymphomas (e.g., Burkitt's lymphoma, diffuse large B cell lymphoma (DLBCL), melanomas (e.g., cutaneous melanoma), carcinomas (e.g., papillary renal cell carcinoma, chromophobe renal cell carcinoma, myelomas (e.g., multiple myeloma), leukemias (e.g., ALL and AML), glioblastoma, astrocytoma, oligodendroglioma, etc.

### Gene Expression and MDM2i Sensitivity

A variety of methods, technologies and procedures as known and used in the art may be employed to assay cancer or non-cancer cell, tissue, or organ samples and specimens for detection of expression levels of genes associated with the MDM2i gene sensitivity signatures of the invention. In an embodiment, the expression levels of the described biomarker genes (such as at least three, or at least four, or all, of the genes listed in FIGS. 1A-1E; in Table 1 herein; or in the gene signature set containing the genes RPS27L, FDXR, CDKN1A and AEN (and optionally MDM2)) in a sample can be determined by quantifying the amount or level of nucleic acid that is transcribed from each biomarker gene. In various aspects, gene expression profiles can be prepared using any quantitative or semi-quantitative method for determining RNA transcript levels in samples. Examples of such methods include, without limitation, hybridization-based assays, such as microarray analysis and similar formats (e.g., Whole Genome DASL Assay, Illumina, Inc., San Diego, Calif.), polymerase-based assays, such as RT-PCR (e.g., TAQMAN®), or real time quantitative reverse transcription PCR (real time qRT-PCR), (e.g., as commercialized by Invitrogen; or Life Technologies), flap-endonuclease-based assays (e.g., INVADER® assay), as well as multiplex assays involving direct RNA (mRNA) capture with branched DNA (QUANTIGENE® ViewRNA, Affymetrix, Santa Clara, Calif.), HYBRID CAPTURE® (Digene, Gaithersburg, Md.), or NCOUNTER® Analysis System (NanoString) as described further herein. Alternatively, or in addition, the level of specific protein translated from mRNA transcribed from a biomarker gene can be determined as described further herein.

The assay format, in addition to determining the gene expression levels for a combination of genes listed in the gene signatures presented in FIGS. 1A-1E, Table 1; and in the MDM2i gene sensitivity signature containing the genes RPS27L, FDXR, CDKN1A and AEN (and optionally MDM2) will also allow for the control of parameters such as intrinsic signal intensity variation between tests. Such controls may include, for example, controls for background signal intensity and/or sample processing, and/or other desirable controls for gene expression quantification across samples. For example, expression levels between samples may be controlled by testing for the expression level of one or more genes, e.g., at least three or at least four genes, that are or are not highly expressed in MDM2i-sensitive cells, or which are generally expressed at similar levels across the population. Such genes may include constitutively expressed genes, as known in the art and described herein. Exemplary assay formats for determining gene expression levels, and thus for preparing gene expression profiles and drug-sensitive are described herein.

### Nucleic Acid Samples

For nucleic acid detection in the methods of the invention, the nucleic acid sample is typically in the form of mRNA or reverse transcribed mRNA (cDNA) obtained or isolated from a cell, tissue, or organ sample or specimen from a cancer or tumor undergoing testing. In some embodiments, the nucleic acids in the sample may be cloned or amplified, generally in a manner that does not bias the representation of the transcripts within a sample. In some embodiments, it may be preferable to use total RNA or polyA+ RNA as a source without cloning or amplification, to avoid additional processing steps. RNA can be isolated from a cancer sample, e.g., a tumor or neoplasm, e.g., a melanoma, lymphoma, or multiple myeloma tumor or neoplasm from a subject, and/or one or more of a sample of adjacent non-tumor tissue from the subject, a sample of tumor-free tissue from a normal or healthy subject, using methods well known to the skilled practitioner, including the use of commercially available kits. Methods of isolating total mRNA are well known in the art and are described in standard textbooks of molecular biology, which provide detailed protocols and guidance, including Ausubel et al., Current Protocols of Molecular Biology, John Wiley and Sons (1997). Methods for RNA extraction from paraffin-embedded tissues are disclosed, for example, in Rupp and Locker, Biotechniques 6:56-60 (1988), and De Andres et al., Biotechniques 18:42-44 (1995). In addition, methods of isolation and purification of nucleic acids are described in numerous academic and commercial sources, nonlimiting examples of which include Molecular Cloning: A Laboratory Manual, 2012, By M. R. Green and J. Sambrook, Cold Spring Harbor Laboratory Press; Current Protocols in Molecular Biology (5^{th} Edition), 2002, F. M. Ausubel et al., John Wiley & Sons, Inc.; Laboratory Techniques in Biochemistry and Molecular Biology, Vol. 24, Chapter 3, Hybridization With Nucleic Acid Probes: Theory and Nucleic Acid Probes, P. Tijssen, Ed., Elsevier Press, New York, 1993 (and later editions). Nucleic acid samples include RNA samples as well as cDNA synthesized from an mRNA sample isolated from a cell or specimen of interest. Such samples also include DNA amplified from the cDNA, and RNA transcribed from the amplified DNA.

For gene expression detection, isolated nucleic acid molecules, e.g., oligonucleotides or probes that include specified lengths of nucleotide sequences, such as the nucleotide sequences of at least three, at least four, or all, of the genes or subsets thereof as listed in the gene signatures of the invention, such as the genes in FIGS. 1A-1E; in Table 1; or in the gene set RPS27L, FDXR, CDKN1A and AEN, are embraced as described herein.

In one example, RNA isolation can be performed using a purification kit, buffer set and protease from commercial manufacturers such as QIAGEN (Valencia, Calif.), according to the manufacturer's instructions. For example, total RNA from cancer cells (e.g., as obtained from a subject with cancer) can be isolated using QIAGEN RNeasy® mini-columns. Other commercially-available RNA isolation kits include MASTERPURE® Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from a biological sample (cancer sample or specimen) can also be isolated, for example, by cesium chloride density gradient centrifugation, as known to those skilled in the art. As discussed herein, in some examples of the detection methods, the expression level of one or more “housekeeping” genes or “internal controls” can also be evaluated. Such controls include any constitutively- or universally-expressed gene (or protein) whose presence enables an assessment of gene (or protein) levels of the disclosed gene expression signature. Such an assessment includes a determination of the overall constitutive level of gene transcription and a control for variations in RNA (or protein) recovery.

### Hybridization-Based Formats, Procedures and Assays

Gene expression profiling for expression of genes of the gene signatures of the invention can be performed using methods that are based on hybridization analysis of polynucleotides, sequencing of polynucleotides, and proteomics-based methods. In some embodiments, mRNA expression levels in a sample are quantified using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992), or quantitative real-time PCR. Alternatively, antibodies that recognize specific duplexes, including DNA duplexes, RNA duplexes, and DNA-RNA hybrid duplexes or DNA-protein duplexes can be used. Bead-based multiplex assays, e.g., the Luminex xMAP® assay, can also be utilized. Without limitation, methods for sequencing-based gene expression analysis include Serial Analysis of Gene Expression (SAGE) and gene expression analysis by massively parallel signature sequencing (MPSS). In one example, RT-PCR can be used to compare mRNA levels in different samples, such as in normal and in cancer tissues to characterize patterns of gene expression levels, to distinguish between closely-related mRNAs and to analyze RNA structure.

In situ hybridization (ISH) provides a method of detecting and comparing expression levels of genes of interest which applies and extrapolates the technology of nucleic acid hybridization to the single cell level. In combination with cytochemistry, immunocytochemistry and immunohistochemistry techniques, ISH allows the morphology of cellular markers to be maintained and identified and further allows localization of sequences to specific cells within populations, such as tissues and blood samples. The hybridization of ISH utilizes a complementary nucleic acid to localize one or more specific nucleic acid sequences in a portion or section of tissue, i.e., in situ, or in the entire tissue (whole mount ISH), if the tissue is small enough. RNA ISH can be used to assay expression patterns (mRNA) in a tissue, such as the expression level of the disclosed genes. In the method, sample cells or tissues are treated to increase their permeability so as to allow a probe, such as a gene-specific probe, to enter the cells. The probe is added to the treated cells, allowed to hybridize at an appropriate temperature, and excess probe is washed away. A complementary probe is labeled to be able to determine the location and quantity of the probe in the tissue undergoing analysis, for example, using autoradiography, fluorescence microscopy or immunoassay. The sample can be any type of sample as described herein, such as a cancer sample or a non-cancer sample. Since the sequences of the genes of interest are known, probes can be appropriately designed to allow the probes to bind specifically to the gene of interest.

In situ PCR is a PCR-based amplification of the target nucleic acid sequences that is carried out prior to ISH detection. For detection of RNA, an intracellular reverse transcription step is introduced to generate complementary DNA (cDNA) from RNA templates prior to in situ PCR. This allows the detection of RNA sequences that are of low copy number. Prior to in situ PCR, the cells or tissue samples are fixed and permeabilized to preserve morphology and to permit access of the PCR reagents to the intracellular sequences that will be amplified. PCR amplification of target sequences is then performed on intact cells in suspension, or directly in cytocentrifuge preparations or tissue sections on glass slides. In the former approach, fixed cells suspended in the PCR reaction mixture are thermally cycled using conventional thermal cyclers. After PCR amplification, the cells are cytocentrifuged onto glass slides to permit visualization of intracellular PCR products by ISH or immunohistochemistry. In situ PCR of cells or tissue samples on glass slides is performed by overlaying the samples with the PCR mixture and applying a coverslip, which is then sealed to prevent evaporation of the reaction mixture. Thermal cycling is performed by placing the glass slides either directly on top of the heating block of a conventional or specially-designed thermal cycler or by using thermal cycling ovens, as known to those having skill in the art. In general, intracellular PCR products are detected by one of two different techniques: indirect in situ PCR by ISH, using PCR-product specific probes, or direct in situ PCR without ISH, through direct detection of labeled nucleotides (such as digoxigenin-11-dUTP, fluorescein-dUTP, ³H-CTP or biotin-16-dUTP), which have been incorporated into the PCR products during thermal cycling.

The SAGE method permits the simultaneous and quantitative analysis of a large number of gene transcripts without the need for providing an individual hybridization probe for each transcript. Briefly, to carry out this type of method, a short sequence tag (about 10-14 base pairs) is generated that contains nucleic acid sequence sufficient information to uniquely identify a transcript, provided that the tag is obtained from a unique position within each transcript. Then, many transcripts are linked together to form long serial molecules that can be sequenced, thus simultaneously providing the identity of the multiple tags. The expression pattern of any population of transcripts can be quantitatively evaluated by determining the abundance of individual tags, and identifying the gene corresponding to each tag (see, e.g., Velculescu et al., Science, 270:484-7, 1995; and Velculescu et al., Cell, 88:243-51, 1997).

In an embodiment, a hybridization-based assay can be used to determine a cancer or tumor sample's MDM2i sensitive gene expression profile, or to determine expression of genes of an MDM2i-sensitive gene signature in accordance with the invention. Nucleic acid hybridization involves contacting a probe and a target sample under conditions in which the probe and its complementary target sequence (if present) in the sample can form stable hybrid duplexes through complementary base pairing. Probes based on the sequences of the genes described herein for preparing expression profiles from cancer, tumor, or neoplasm samples undergoing analysis can be prepared by any suitable method. A probe is a nucleic acid capable of binding to a target nucleic acid of complementary sequence through one or more types of chemical bonds, typically through complementary base pairing and hydrogen bond formation. A probe may include natural nucleotide bases (i.e., A, U, C, or T) or modified nucleotide bases (e.g., 7-deazaguanosine, inosine, etc.), or locked nucleic acid (LNA). In addition, the nucleotide bases comprising probes may be joined by a linkage other than a phosphodiester bond, so long as the bond does not interfere with hybridization. Thus, probes may be peptide nucleic acids in which the constituent bases are joined by peptide bonds rather than phosphodiester linkages.

Oligonucleotide probes for hybridization-based assays will be of sufficient length or composition (including nucleotide analogs) to hybridize (or bind) specifically to appropriate complementary nucleic acids (e.g., exactly or substantially complementary RNA transcripts (mRNA) or cDNA). In general, the oligonucleotide probes are linear and will be at least 8, at least 10, at least 12, at least 14, at least 16, at least 18, at least 20, at least 25, or at least 30 nucleotides (consecutive nucleotides) in length. In some cases, longer probes, e.g., at least 30, at least 40, at least 45, at least 50 nucleotides, or up to about 200 nucleotides in length can be used. These sequences can be obtained from any region of the disclosed genes, e.g., from the at least three, at least four, at least five, at least six, at least seven, at least eight, at least nine, at least ten, or all, of the genes presented in FIGS. 1A-1E; Table 1; or at least three, or all, of the genes in the gene set containing the genes RPS27L, FDXR, CDKN1A and AEN (and optionally MDM2). In some embodiments, complementary hybridization between a probe nucleic acid and a target nucleic acid may include minor mismatches (e.g., one, two, or three mismatches) that can be accommodated by reducing the stringency of the hybridization media to achieve the desired detection of the target polynucleotide sequence. Of course, the probes may be perfect matches with the intended target probe sequence, for example, the probes may each have a probe sequence that is perfectly complementary to a target sequence (e.g., a sequence of a gene listed in FIGS. 1A-1E; Table 1; or in the RPS27L, FDXR, CDKN1A and AEN (and optionally MDM2) gene signature according to the invention.

The nucleic acids that do not form hybrid duplexes are washed away, thereby allowing the hybridized nucleic acids to be detected, typically via detection of an attached detectable label. One or more labels attached to the sample nucleic acids can be used to detect hybridized nucleic acids. The labels may be incorporated by a variety of means that are conventionally known to those of skill in the art. See, e.g., US 2012/0264639. Methods of physically detecting the binding of complementary strands of nucleic acid molecules include, without limitation, DNase I or chemical footprinting, gel shift and affinity cleavage assays, dot blot, Northern blot, and light absorption detection methods. In one exemplary method, a change in light absorption of a solution containing an oligonucleotide (or an analog thereof) and a target nucleic acid is observed at a spectrophotometric wavelength of 220 to 300 nm as the temperature is increased. If the oligonucleotide or analog has bound to its target, a rapid increase in absorption occurs at a characteristic temperature as the oligonucleotide (or analog) and its target disassociate from each other, or melt. In another example, the method involves the detection of a signal, e.g., a detectable label, present on one or both nucleic acid molecules (or antibody or protein as appropriate). Methods of detecting binding of an antibody to a protein are routine, such as immunohistochemical or Western blot techniques.

As understood by the skilled practitioner, nucleic acids can be denatured by increasing the temperature or decreasing the salt concentration of the buffer containing the nucleic acids. Under low stringency conditions (e.g., low temperature and/or high salt concentration) hybrid duplexes (e.g., DNA:DNA, RNA:RNA, or RNA:DNA) will form even in cases in which the annealed sequences are not perfectly complementary. Thus, specificity of hybridization is reduced at lower stringency. Conversely, at higher stringency (e.g., higher temperature or lower salt concentration), successful hybridization tolerates fewer mismatches. One of skill in the art will recognize that hybridization conditions may be selected to provide any degree of stringency. A hybridization-based assay is generally conducted under so-called stringent conditions in which the probe(s) will hybridize to their intended target subsequence with only non-substantial hybridization to other or irrelevant sequences, such that the difference can be identified. Stringent conditions are sequence-dependent and can vary under different circumstances. For example, longer probe sequences generally hybridize to perfectly complementary sequences (over less than fully complementary sequences) at higher temperatures. In general, stringent conditions may be selected to be about 5° C. lower than the thermal melting point (Tm) for the specific sequence at a defined ionic strength and pH. Examples of stringent conditions include those in which the salt concentration is at least about 0.01 to 1.0 M Na⁺ ion concentration (or other salts) at pH 7.0 to 8.3, and the temperature is at least about 30° C. for short probes (e.g., 10 to 50 nucleotides). Desired hybridization and stringency conditions may also be achieved through the addition of agents such as formamide or tetramethyl ammonium chloride (TMAC).

In certain examples, hybridization is performed under low stringency conditions, such as 6×SSPET at 37° C. (0.005% Triton X-100), to ensure hybridization; subsequent washes are then performed under higher stringency conditions (e.g., 1×SSPET at 37° C.) to eliminate mismatched hybrid duplexes. Successive washes can be performed at increasingly higher stringency (e.g., down to as low as 0.25×SSPET at 37° C. to 50° C.) until a desired level of hybridization specificity is obtained. Hybridization specificity may be evaluated by comparing hybridization to the test probes with hybridization to the various controls that may be present, as described below (e.g., expression level control, normalization control, mismatch controls, and the like). As understood by the skilled practitioner, there is frequently a tradeoff between hybridization specificity (stringency) and signal intensity. Thus, in an example, the wash is performed at the highest stringency that produces consistent results and that provides a signal intensity greater than approximately 10% of the background intensity. The hybridized array can be washed at successively higher stringency solutions and evaluated between each wash. Analysis of the data sets generated reveals a wash stringency above which the hybridization pattern is not appreciably altered and which provides adequate signal for the particular oligonucleotide probes of interest.

The hybridization-based assay may also employ mismatch controls for the target sequences, and/or for expression level controls or for normalization controls. Mismatch controls are probes designed to be identical to their corresponding test or control probes, except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferably, mismatch probes contain a central mismatch. Thus, for example, in the case in which a probe is a 20-mer, a corresponding mismatch probe will have the identical sequence except for a single base mismatch (e.g., substituting a G, a C or a T for an A) at any of positions 6 through 14 (the central mismatch). Mismatch probes thus provide a control for non-specific binding or cross hybridization to a nucleic acid in the sample other than the target to which the probe is directed. For example, if the target nucleic acid is present in the sample, then the probes that perfectly match should provide a more intense signal than the probes that are mismatched. The difference in intensity between the perfect match and the mismatch probe aids in providing a reliable measure of the concentration of the hybridized material.

A number of hybridization assay formats are known and are suitable for use in conjunction with the methods of the invention. Such hybridization-based formats include solution-based and solid support-based assay formats. Solid supports containing oligonucleotide probes designed to detect differentially expressed, e.g., highly expressed, genes (e.g., as listed in FIGS. 1A-1E; in Table 1; and in the gene signature having the components RPS27L, FDXR, CDKN1A and AEN (and optionally MDM2), as described herein) can be filters, polyvinyl chloride dishes, particles, beads, microparticles or silicon or glass based chips, etc. Any solid surface to which oligonucleotides can be directly or indirectly bound, either covalently or non-covalently, can be used. Bead- or microsphere-based assays are described, for example, in U.S. Pat. Nos. 6,355,431, 6,396,995, and 6,429,027. Chip-based assays are described, for example, in U.S. Pat. Nos. 6,673,579, 6,733,977, and 6,576,424 and are described further herein. Techniques and general methods for preparing and using polynucleotide microarrays to measure expression of biomarker genes are described, for example, in US Pre-Grant Publication No. US 2011/0015869 and are described elsewhere herein.

As will be appreciated by the skilled practitioner, background signals may need to be controlled for when using hybridization-based assays. The terms “background” or “background signal intensity” refer to hybridization signals which result from non-specific binding or other interactions between the labeled target nucleic acids and components of the oligonucleotide array (e.g., the oligonucleotide probes, control probes, the array substrate, etc.). Background signals can also be produced by intrinsic fluorescence of the array components themselves. A single background signal can be calculated for the entire array, or a different background signal can be calculated for each location of the array. In way of example, background can be calculated as the average hybridization signal intensity for the lowest 5% to 10% of the probes in the array. Alternatively, background may be calculated as the average hybridization signal intensity produced by hybridization to probes that are not complementary to any sequence found in the sample (e.g. probes directed to nucleic acids of the opposite sense or to genes not found in the sample, such as bacterial genes in cases in which the sample is mammalian (human) nucleic acids). Background can also be calculated as the average signal intensity produced by regions or locations of the array that lack any probes at all. In addition, background hybridization signals may be controlled for using one or a combination of known approaches, including one or a combination of the above-described approaches.

The hybridization-based assays can include, in addition to the “test probes” (e.g., those that bind the target sequences of interest, such as those comprising the genes in the gene signatures of the invention, for example, as are listed in FIGS. 1A-1E; in Table 1; or in the set of RPS27L, FDXR, CDKN1A and AEN (and optionally MDM2) genes), one or a combination of control probes, such as normalization controls, expression level controls, and mismatch controls. For example, when determining the levels of gene expression in patient or control samples, the expression values may be normalized to control between the test and control samples, e.g., by determining the level of expression of one or more constitutively expressed gene in each sample, for example, by mRNA analysis. Typically, expression level control probes have sequences complementary to subsequences of constitutively expressed human housekeeping genes, as defined herein, which generally would not exhibit increased expression in drug-sensitive versus drug-insensitive samples.

Other controls can involve, for example, using probes designed to be complementary to a labeled reference oligonucleotide added to the nucleic acid sample to be assayed. The signals obtained from the controls after hybridization provide a control for variations in the hybridization conditions, label intensity, “reading” efficiency and other factors that can cause the signal of an exact hybridization to vary between arrays. In one embodiment, signals (e.g., fluorescence intensity) read from all other probes in the array are divided by the signal (e.g., fluorescence intensity) from the control probes, thereby normalizing the measurements. Exemplary probes for normalization are selected to reflect the average length of the other probes (e.g., test probes) present in the array, however, they may be selected to cover a range of lengths. The control(s) can also be selected to reflect the (average) base composition of the other probes in the array. In some cases, the assay will utilize one or a few control probes, which are selected to hybridize without secondary structure and without hybridizing to any potential targets.

### Reverse Transcription Polymerase Chain Reaction (RT-PCR)

In some embodiments, reverse transcription polymerase chain reaction (RT-PCR) can be employed. RT-PCR is a sensitive method for the detection of mRNA, including low-abundant mRNAs frequently present in clinical samples. The application of fluorescence techniques to RT-PCR, combined with suitable instrumentation, has resulted in quantitative RT-PCR methods that combine amplification, detection and quantification in a closed system. For example, two commonly used quantitative RT-PCR techniques are the TAQMAN® RT-PCR assay (ABI, Foster City, USA) and the LIGHTCYCLER® assay (Roche Applied Sciences, Indianapolis, Ind.).

Methods for quantifying mRNA, such as RT-PCR are well known in the art. By way of example, extracted RNA can be reverse-transcribed using a GENEAMP® RNA PCR reagent kit (Perkin Elmer, CA, USA), following the manufacturer's instructions. In some embodiments, gene expression levels can be determined using a gene expression analysis technology that measures mRNA in solution. Examples of such gene expression analysis technologies include, but are not limited to, RNAscope™, RT-PCR, NANOSTRING®, QUANTIGENE®, gNPA®, microarray, and sequencing. NANOSTRING® methods, for example, NCOUNTER™ Digital Gene Expression System (Seattle, Wash.) use labeled reporter molecules, referred to as labeled “nanoreporters,” that can bind to individual target molecules (See, e.g., U.S. Pat. No. 7,473,767; Geiss, Nature Biotechnology, 26, 317-325, 2008; WO 2007/076128; and WO 2007/076129). Based on the label codes of the nanoreporters, the binding of the nanoreporters to target molecules results in the identification of the target molecules.

According to an embodiment of the invention, the preparation of a patient's gene expression profile from a sample or specimen, or the preparation of drug-sensitive (or drug-resistant) profiles involves performing real-time, quantitative PCR (TAQMAN® qRT-PCR) assays with sample-derived RNA and control RNA. The TAQMAN® assay is known and used by those having skill in the pertinent art; it is also described, for example, in Holland, et al., 1991, PNAS 88:7276-7280. In addition, versions of the TAQMAN® assay are described in U.S. Pat. No. 5,210,015 (Gelfand et al.), and in U.S. Pat. No. 5,491,063 (Fisher, et al.). TAQMAN® RT-PCR can be performed using commercially-available methods and systems, which can include a thermocycler, laser, charge-coupled device (CCD) camera, and computer. The system amplifies samples in a 96-well format on a thermocycler. Quantitative RT-PCR measures PCR product accumulation through a dual-labeled fluorogenic probe (e.g., TAQMAN® probe). During amplification, a laser-induced fluorescent signal is collected in real-time through fiber optics cables for all 96 wells, and detected at the CCD. The system includes software for running the instrument and for analyzing the data.

The TAQMAN® methodology and detection assay system offer advantages, such as the efficient handling of large numbers of samples without cross-contamination; consequently, it is highly adaptable for robotic sampling. Another of its advantages is the potential for multiplexing. That is, because different fluorescent reporter dyes can be used to construct probes, the expression of several different genes associated with MDM2i drug sensitivity can be assayed in the same PCR reaction, thereby leading to cost reductions compared to performing each reaction/test individually. Thus, the TAQMAN® assay format may be preferable in cases in which the gene expression profile from a patient's sample, and the corresponding MDM2i-sensitivity profiles involve the expression levels of about 40 or fewer, or about 20 or fewer, or about 10 or fewer, or about 7 or fewer, or about 5 or fewer, or about 4 or fewer genes, for example, the at least three, at least four, or all, of the genes listed in one or more of FIGS. 1A-1E, Table 1, or the genes RPS27L, FDXR, CDKN1A and AEN (and optionally MDM2), comprising gene signatures of the invention.

To minimize errors and the effects of sample-to-sample variation, RT-PCR can be performed using an internal standard. Optimally, an internal standard is expressed at a constant level among different tissues and is unaffected by an experimental treatment. Typical RNAs used to normalize patterns of gene expression are mRNAs for the housekeeping genes, such as GAPDH, β-actin and 18S ribosomal RNA. RT-PCR is compatible with both quantitative competitive PCR, in which an internal competitor for each target sequence is used for normalization and quantitative comparative PCR, in which a normalization gene contained within the sample, or a housekeeping gene, for RT-PCR is used. (e.g., Heid et al., 1996, Genome Research, 6:986-994). Quantitative PCR, related probes and quantitative amplification procedures are described, for example, in U.S. Pat. No. 5,538,848; in U.S. Pat. No. 5,716,784 and in U.S. Pat. No. 5,723,591. Quantitative PCR can be carried out in microtiter plates using instruments designed for this purpose (PE Applied Biosystems, Foster City, Calif.).

Gene expression levels can be quantified using fixed, paraffin-embedded tissues as the RNA source following mRNA isolation, purification, primer extension and amplification, as described, for example in several publications, e.g., Godfrey et al., J. Mol. Diag. 2:84-91, 2000; Specht et al., Am. J. Pathol. 158:419-29, 2001. In brief, such a process begins with cutting sections of paraffin-embedded cancer tissue samples or adjacent non-cancerous tissue about 10 μm thick. The RNA is extracted, and protein and DNA are removed. Alternatively, RNA is isolated directly from a cancer sample or other tissue sample. After analysis of the RNA concentration, RNA repair and/or amplification steps can be included, if necessary or desired, and RNA is reverse transcribed using gene specific promoters followed by RT-PCR.

In some embodiments, the primers used for the amplification are selected so as to amplify a unique segment of the gene of interest (such as mRNA encoding at least 3, 4, 5, 6, or more, or all, of the gene signature genes listed in FIGS. 1A-1E; or in other gene signatures provided by the invention, such as e.g., at least 3, 4, 5, 6 or more, or all, of the genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1, and/or XPC; or at least 3, or all, of the genes MDM2, CDKN1A, ZMAT3, DDB2, FDXR, RPS27L, BAX, RRM2B, SESN1, CCNG1, XPC, TNFRSF10B and AEN; or at least 3, or all, of the genes RPS27L, FDXR, CDKN1A and AEN (and optionally MDM2). In some embodiments, the expression levels of other genes are also detected, for example, one or more control or housekeeping genes. Primers that can be used to amplify one or more of the gene signature may be commercially-available or can be designed and synthesized according to well-known and conventionally used methods. An alternative quantitative nucleic acid amplification procedure is described in U.S. Pat. No. 5,219,727, which relates to a procedure in which the amount of a target sequence in a sample is determined by simultaneously amplifying the target sequence and an internal standard nucleic acid segment. The amount of amplified DNA from each segment is determined and compared to a standard curve to determine the amount of the target nucleic acid segment present in the sample prior to amplification.

In other embodiments, methods for use in accordance with the invention can employ detection and quantification of RNA levels in real-time using nucleic acid sequence based amplification (NASBA) combined with molecular beacon detection molecules. NASBA is described, e.g., by Compton J., 1991, Nucleic acid sequence-based amplification, Nature, 350(6313):91-2. NASBA, a single-step isothermal RNA-specific amplification method, comprises the following steps: An RNA template is introduced into a reaction mixture, wherein the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; T7 RNA polymerase binds double-stranded DNA and produces a complementary RNA strand which can be used again in step 1, providing a cyclic reaction.

In other embodiments, the assay format may be a flap endonuclease-based format, such as the INVADER® assay (Hologic™, formerly Third Wave Technologies, Madison, Wis.). In brief, the INVADER® method is composed of two simultaneous isothermal reactions. In the first reaction, two oligonucleotides, a probe and an INVADER® oligonucleotide, associate with a specific region of the target DNA, such as DNA obtained from a patient's tumor sample. If the desired sequence is present, an overlapping structure is created with the probe and the Invader® oligonucleotide on the target. Proprietary CLEAVASE® enzymes specifically cleave the primary probes that form overlapping structures with the INVADER® oligonucleotide, releasing the 5′ flaps plus one nucleotide. More specifically in the primary reaction, multiple probe molecules are cleaved per target molecule, and the signal generated from the cleaved 5′ flap is amplified. The probes cycle rapidly on and off the target; each time an intact probe molecule binds to the specific target in the presence of the INVADER® oligonucleotide, the overlapping substrate is formed and cleavage can occur. The number of flaps released is relative to the amount of target in the sample, allowing for quantitative detection of genes. Released flaps from the primary reaction serve as INVADER® oligonucleotides in a second, simultaneous, overlapping cleavage reaction on a labeled, synthetic oligonucleotide, called the fluorescence resonance energy transfer (FRET) probe. Cleavage of the FRET probe results in the generation of a fluorescent signal. Using two different 5′ flap sequences and their complementary FRET oligonucleotides with non-overlapping fluorophores allows for two distinct sequences to be detected in a single well. Each released 5′ flap from the primary reaction cycles on and off the cleaved and uncleaved FRET probes, thereby enabling cleavage of many FRET probes in the secondary reaction to further amplify the target-specific signal. In still other embodiments, the assay format may utilize direct mRNA capture with branched DNA (QUANTIGENE™, Affymetrix/Panomics, Santa Clara, Calif.) or HYBRID CAPTURE™ (Digene Corp., Gaithersburg, Md.). The design of probes suitable and appropriate for hybridizing to a particular target nucleic acid and for configuration for any appropriate nucleic acid detection assay, is well known and practiced routinely by those having skill in the pertinent art.

### Arrays and Microarrays

In some embodiments, gene expression levels are identified or confirmed using microarray platforms and techniques. In array and microarray methods, the nucleic acid sequences of interest (including cDNAs and oligonucleotides) are overlaid, plated, or arrayed, on a substrate, such as a microchip. The arrayed sequences are then hybridized with isolated nucleic acids (such as cDNA or mRNA) from cells or tissues of interest. As an example, expressed genes in the MDM2i sensitivity gene signatures can be measured in either fresh or paraffin-embedded cancer/tumor/neoplasm tissue, using microarray technology.

Similar to the RT-PCR method, for microarray technology, the mRNA source is typically total RNA isolated from the cancer or neoplasm, and, optionally, from corresponding noncancerous tissue, and normal tissues or cell lines. In a specific example of the microarray technique, PCR amplified inserts of cDNA clones are applied to a substrate in a dense array. In some examples, the array includes at least one probe specific to each of, for example, at least three, at least four, at least five, at least six, at least seven, at least eight, at least nine, at least ten, or all, of the disclosed genes in the gene signatures according to the invention, such as those provided in FIGS. 1A-1E or in Table 1. In some aspects, oligonucleotide probes specific for the nucleotide sequences of each of the at least three, at least four, at least five, at least six, at least seven, at least eight, at least nine, at least ten, or all, of the genes listed in FIGS. 1A-1E, Table 1, namely, BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1, and/or XPC, or at least three, or all, of the genes MDM2, CDKN1A, ZMAT3, DDB2, FDXR, RPS27L, BAX, RRM2B, SESN1, CCNG1, XPC, TNFRSF10B and AEN, or at least three, or all, of the genes RPS27L, FDXR, CDKN1A and AEN (and optionally MDM2) are arrayed on the substrate. The arrayed sequences can include, consist essentially of, or consist of these nucleotide sequences. The nucleic acids in the microarrays are suitable for hybridization, e.g., under stringent conditions.

Labeled cDNA probes can be generated, for example, via incorporation of fluorescent nucleotides by reverse transcription of RNA extracted from tissues of interest. Labeled cDNA probes that are applied to the array hybridize with specificity to each spot of DNA applied to the array. After stringent washing to remove non-specifically bound nucleic acid probe, the array is scanned by a suitable detection method, such as confocal laser microscopy or by use of a CCD camera. The quantification of hybridization of each arrayed element allows the corresponding mRNA abundance to be assessed. With dual color fluorescence, separately-labeled cDNA probes generated from two sources of RNA can be hybridized pairwise to the array. The relative abundance of the transcripts from the two sources corresponding to each specified gene is thus able to be determined simultaneously. The miniaturized scale of the hybridization affords a convenient and rapid evaluation of the expression levels and expression level patterns in the cancer or tumor sample of at least three, at least four, or all, of the genes listed in FIGS. 1A-1E, Table 1, as well as at least three, or all, of the genes RPS27L, FDXR, CDKN1A and AEN (and optionally MDM2), whose expression is indicative of MDM2i sensitivity according to the invention.

A high throughput method for obtaining information about gene expression is provided by the nucleic acid microarray in which a transparent support, such as a microscope slide, containing dozens to hundreds to thousands or more of immobilized nucleic acid samples is hybridized in a manner that is similar to hybridization in Northern and Southern blots. An optimum support allows effective immobilization of nucleic acid sequences (i.e., probes) onto its surface, as well as efficient and effective hybridization of target nucleic acid sequences with the probe. Following hybridization with dye-tagged nucleic acids, the array is “read” using a laser scanner to stimulate (fluoresce) the dye attached to the nucleic acid targets hybridized to the probes on the support. A motorized stage executes a programmed comb scan pattern that sequentially traverses the array in the X direction, and then steps a pixel width in the Y direction, producing a bi-directional raster pattern. Part of the dye fluorescence is captured by the scanner objective and is filtered into red and green signals that are routed to each respective photomultiplier tube (PMT) where they are converted to electrical signals that are amplified, filtered and sampled by an analog-to-digital (A/D) converter. The scanner software converts the A/D converter output into a high-resolution image on which the pixel intensity of each spot is proportional to the number of dye molecules, and to the number of probe nucleic acids that are hybridized with the target nucleic acids on the array. Addressable arrays are usually computer readable, in that a computer can be programmed to correlate a particular address on the array with information e.g., hybridization or binding data, about the sample at that position, including signal intensity. In some examples of computer readable formats, the individual features in the array are arranged regularly, for instance in a Cartesian grid pattern, which is correlated to address information by the computer.

Microarray analysis can be performed using commercially-available systems, kits and equipment of choice, following the manufacturer's instructions and protocols, e.g., as provided with Affymetrix GENECHIP® technology (Affymetrix, Santa Clara, Calif.) or Agilent microarray technology (Agilent Technologies, Santa Clara, Calif.). Alternatively and as described elsewhere herein, the assay format may employ the NCOUNTER® Analysis System (NanoString® Technologies) and methodology as described, e.g., in G. K. Geiss et al., 2008, Direct Multiplexed Measurement of Gene Expression with Color-Coded Probe Pairs, Nat. Biotechnol., 26(3):317-25. The system uses molecular “barcode” technology and single molecule imaging to detect and count hundreds of unique mRNA transcripts in a single reaction. Unlike other methods, the protocol does not include any amplification steps that might introduce bias to the results.

In a preferred embodiment, the expression of at least three, at least four, or all, of the genes in FIGS. 1A-1E, in Table 1, or in the set of genes including RPS27L, FDXR, CDKN1A and AEN (and optionally MDM2) in a cancer or tumor sample or specimen is assessed, evaluated, or measured using microarrays or gene chip technology, such as, e.g., Affymetrix GENECHIP® DNA microarrays, provided by Affymetrix (Santa Clara, Calif.). Such arrays provide a maximum number of highly specific and sensitive probes per chip and good detection capability. As will be appreciated by the skilled practitioner in the art, a procedure to make gene expression comparable using nucleic acid arrays can involve the approach of global normalization. In this approach, the averages of the expression distributions (expression levels for all genes within the DNA array) across arrays are set to be equal. This widely used approach follows from the assumption that while a sample's genes can be differentially expressed, the amount of transcription is essentially similar across samples. Thus, global normalization utilizes expression signals of all of the probes on the microarray chip and adjusts for the median signal value among chips.

It will be understood, that the determination and measurement of gene expression of the genes of the MDM2i sensitivity gene signatures of the invention are not limited either by a particular method of analysis or by a particular approach for normalizing gene expression levels. For example, while global normalization may used in the practice of the methods of the invention for normalizing to the average gene expression of the entire array, normalization using housekeeping genes can also be utilized for normalizing to the average expression of the housekeeping genes used.

Thus, it will be apparent that any number of array designs are suitable for the practice of the invention. An array for use with the invention will typically include a number of test probes that specifically hybridize to the sequences of interest. That is, the array will include probes designed to hybridize to any region of the genes listed in FIGS. 1A-1E, Table 1, or in any of the gene signatures described herein. In instances where the gene reference in the gene signatures of the invention may be an EST, probes may be designed from that sequence, or from other regions of the corresponding full-length transcript, that may be available in any of the public sequence databases. Methods of producing probes for a given gene or genes can be found in, for example, US 2012/0264639. Computer software is also commercially available for designing specific probe sequences. Typically, the array will also include one or more control probes, such as mismatch probes, or probes specific for one or more constitutively expressed genes, thereby allowing data from different hybridizations to be compared.

The ordered arrangement of molecules, i.e., “features”, of microarrays allows a very large number of analyses on a sample at one time. For example, in some arrays, one or more molecules (such as an oligonucleotide probe or an antibody) occur on the array a plurality of times (such as two times, for example) to provide internal controls. The number of addressable locations on the array can vary, for example, from at least 4, to at least 9, at least 10, at least 14, at least 15, at least 20, at least 30, at least 40, at least 50, at least 75, at least 100, at least 150, at least 200, at least 300, at least 500, least 550, at least 600, at least 800, at least 1000, at least 10,000, or more. In some cases, an array includes 3-200 addressable locations, such as 3-40, 3-50, or 3-177 addressable locations. In particular examples, an array consists essentially of probes or primers or antibodies (such as those that permit amplification or detection) specific for some or all of the genes of the gene signatures of the invention, e.g., at least three, at least four, or all, of the genes in FIGS. 1A-1E; the genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and/or XPC; the genes MDM2, CDKN1A, ZMAT3, DDB2, FDXR, RPS27L, BAX, RRM2B, SESN1, CCNG1, XPC, TNFRSF10B and AEN; or the genes RPS27L, FDXR, CDKN1A and AEN (and optionally MDM2), and in some examples, also 1 to 10 control molecules (such as probes, primers, or antibodies addressable to housekeeping genes).

Protein-based arrays include probe molecules that are, or that include, proteins, or target molecules that are or include proteins. In some cases, the arrays include nucleic acids to which proteins are bound, or vice versa. In examples, an array contains antibodies to at least three, at least four, at least five, at least 10, different molecules associated with genes of the MDM2i sensitive gene signatures of the invention, and in some examples also 1 to 10 housekeeping genes.

In an embodiment, polynucleotide microarrays can be used to measure the expression of the gene biomarkers of the MDM2i sensitivity gene signatures of the invention. The microarrays provided by the invention may comprise oligonucleotide or cDNA probes that are hybridizable (specifically hybridizable) to at least three, or at least four, or all, of the genes of FIGS. 1A-1E; of Table 1; or of at least three genes of the gene set including RPS27L, FDXR, CDKN1A and AEN (and optionally MDM2), which are indicative of sensitivity of cancer cells and samples to one or more MDM2 inhibitors compared to a control. Expression of each of the genes can be assessed simultaneously. The invention provides polynucleotide arrays comprising probes to at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, up to 177, of the genes or subsets of genes constituting the MDM2i gene sensitivity signature biomarkers of FIGS. 1A-1E, Table 1, and of other gene signatures of the invention, which are differentially expressed, e.g., increased in expression, in cancers and tumors sensitive to MDM2i treatment, as well as probes to one or more control genes. In a specific embodiment, the microarray is a screening or scanning array, for example, as described in WO 2002/18646 to Altschuler et al.; in WO 2002/16650 to Scherer et al; and in US 2011/0015869. In brief, the screening and scanning arrays comprise regularly-spaced, positionally addressable probes derived from genomic nucleic acid sequence, both expressed and unexpressed.

In some embodiments, the array contains probes, primers, or antibodies specific for at least 3, at least 4, at least 5, at least 6, at least 8, at least 10, or all, independently and inclusive, of the gene signature component genes as listed in FIGS. 1A-1E; in Table 1; or, more specifically, at least 3, at least 4, at least 5, at least 6, at least 8, at least 10, or all of the following gene signature genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1, and/or XPC); or at least three, or all, of the genes in the gene signature containing MDM2, CDKN1A, ZMAT3, DDB2, FDXR, RPS27L, BAX, RRM2B, SESN1, CCNG1, XPC, TNFRSF10B and AEN; or at least three, or all, of the genes in the gene signature containing RPS27L, FDXR, CDKN1A and AEN (and optionally MDM2), or the proteins encoded by these genes. In some embodiments, the array further includes one or more control probes, primers, or antibodies. Nonlimiting examples of control probes include those for the GAPDH, β-actin and 18S RNA genes, or antibodies that recognize the proteins encoded by these genes. Optionally, and/or optimally, the cancer or tumor types show consistent TP53 and/or p53-dependent expression in vitro and in vivo.

### Substrates for Arrays

An array substrate or solid support can be formed, for example, from an organic polymer. Suitable materials for the solid support include, but are not limited to, polypropylene, polyethylene, polybutylene, polyisobutylene, polybutadiene, polyisoprene, polyvinylpyrrolidine, polytetrafluroethylene, polyvinylidene difluoride, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, ethyleneacrylic acid, thylene methacrylic acid, and blends of copolymers thereof, e.g., as in U.S. Pat. No. 5,985,567 and in published US Application No. US 2011/0206703.

General characteristics and parameters of materials that are suitable for forming the solid support or substrate include, without limitation, amenability to surface activation so that upon activation, the surface of the support is capable of covalently attaching a biomolecule such as an oligonucleotide or antibody thereto; amenability to “in situ” synthesis of biomolecules; chemically inertness so that the areas on the support that not occupied by the oligonucleotides or proteins (such as antibodies) are not amenable to non-specific binding, or if/when non-specific binding should occur, such materials can be readily removed from the surface without removing bound oligonucleotides or proteins (such as antibodies) of interest. For example, a surface activated organic polymer used as the solid support surface is a polypropylene material aminated via radio frequency plasma discharge. Other reactive groups can also be used, such as carboxylated, hydroxylated, thiolated, or active ester groups.

### Formats for Arrays

A number of array formats can be employed for use with the invention. An array format can include one to which the solid support can be affixed, for example, a microtiter plate (e.g., multi-well plates), test tubes, inorganic sheets, dipsticks, and the like. When the solid support is a polypropylene thread, one or more polypropylene threads can be affixed to a plastic dipstick-type device; alternatively, polypropylene membranes can be affixed to glass slides. No particular format per se is required. At a minimum, the solid support is optimally affixed to the array format without affecting the functional behavior of the solid support or any biopolymer absorbed thereon, and the format (such as the dipstick or slide) should be unreactive with (stable to) any materials into which the device is introduced (such as clinical samples and reaction solutions).

The arrays for use in the invention can be prepared in several ways. As an example, oligonucleotide or protein sequences are synthesized separately and then are attached to a solid support (see, e.g., U.S. Pat. No. 6,013,789). As another example, sequences are synthesized directly onto the support to provide the desired array (see, e.g., U.S. Pat. No. 5,554,501 or US 2011/0206703). Suitable methods for covalently coupling oligonucleotides and proteins to a solid support and for directly synthesizing the oligonucleotides or proteins onto the support are known to and practiced by those having skill in the pertinent field. For guidance; a summary of suitable methods can be found, e.g., in Matson et al., 1994, Anal. Biochem. 217:306-10. In another example, oligonucleotides are synthesized onto the support using conventional chemical techniques for preparing oligonucleotides on solid supports, e.g., as provided in PCT publications WO 85/01051 and WO 89/110977, or U.S. Pat. No. 5,554,501.

An illustrative, yet nonlimiting example is a linear array of oligonucleotide or antibody bands, generally referred to in the art as a dipstick. Another suitable format includes a two-dimensional pattern of discrete cells (such as 4096 squares in a 64 by 64 array). As is appreciated by those skilled in the art, other array formats including, but not limited to, slot (rectangular) and circular arrays, e.g., as in U.S. Pat. No. 5,981,185, or a multi-well plate. As another example, the array is formed on a polymer medium, which is a thread, membrane or film (such as an immunochromatographic medium or membrane). An example of an organic polymer medium is a polypropylene sheet having a thickness on the order of about 1 mil. (0.001 inch) to about 20 mil. The thickness of the film is not critical and can be varied over a fairly broad range. The array can include biaxially oriented polypropylene (BOPP) films, which, in addition to their durability, exhibit low background fluorescence. The array formats contemplated for use herein can constitute various types of formats.

Suitable arrays for use with the gene signatures and of the invention, as well as companion diagnostics related thereto, can be generated using automated processes and/or devices to synthesize oligonucleotides in the cells of the array by laying down the precursors for the four nucleotide bases in a predetermined pattern. Briefly and by way of example, a multiple-channel automated chemical delivery system is employed to create oligonucleotide probe populations in parallel rows (corresponding in number to the number of channels in the delivery system) across the substrate, such as a polypropylene support. Following completion of oligonucleotide synthesis in a first direction, the substrate can then be rotated by 90° to permit synthesis to proceed within a second set of rows that are now perpendicular to the first set. This process creates a multiple-channel array whose intersection generates a plurality of discrete cells. The oligonucleotides can be bound to the polypropylene support either via the 3′ end of the oligonucleotide or via the 5′ end of the oligonucleotide. In an example, the oligonucleotides are bound to the solid support by the 3′ end. As would be understood by the skilled practitioner in the art, it can be readily determined by the practitioner whether the use of the 3′ end or the 5′ end of the oligonucleotide is suitable for binding to the solid support. In general, the internal complementarity of an oligonucleotide probe in the region of the 3′ end and the 5′ end determines binding, or binding orientation, to the support. As mentioned herein, oligonucleotide probes or antibodies on the array may include one or more labels that permit the detection of hybridization complexes comprising oligonucleotide probe/target sequences or antibody/protein complexes.

### Detection of Protein Expression Levels

In some aspects, the expression level in a cancer or tumor sample of, for example, at least three, at least four, at least five, at least six, at least seven, at least eight, at least nine, or at least ten, or all, of the proteins encoded by the genes disclosed in accordance with the described MDM2i sensitivity gene signatures are analyzed. In particular examples, the expression levels in a sample of three or more, four or more, five or more (e.g., six or more, ten or more, 30 or more, 37 or more, 38 or more, 40 or more, or all) of the proteins encoded by the genes in the MDM2i gene sensitivity signatures of the invention are analyzed. In an embodiment, the proteins encoded by at least three, at least four, at least five, at least six, or all, of the MDM2i gene signature genes of FIGS. 1A-1E are analyzed, and antibodies directed to the protein products of these genes are used. In an embodiment, the proteins encoded by at least three, at least four, at least five, at least six, or all, of the MDM2i gene signature genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1, and/or XPC are analyzed, and antibodies directed to the protein products of these genes are used. In an embodiment, the proteins encoded by at least three, or all, of the genes MDM2, CDKN1A, ZMAT3, DDB2, FDXR, RPS27L, BAX, RRM2B, SESN1, CCNG1, XPC, TNFRSF10B and AEN are analyzed. In an embodiment, the proteins encoded by at least three, or all, of the genes RPS27L, FDXR, CDKN1A and AEN (and optionally MDM2) are analyzed, and antibodies directed to the protein products of these genes are used.

Suitable samples from which to detect protein levels include biological samples containing proteins obtained from a cancer or tumor (such as, for example, a melanoma or a multiple myeloma tumor or neoplasm) of a subject, from non-cancer tissue of the subject, and/or protein obtained from one or more samples obtained from cancer-free or normal subjects. Detecting a difference in the levels of, or alterations in the amounts of, for example, at least three or at least four (or more, up to all) of the proteins encoded by the genes within the gene signatures of the invention (i.e., the genes in FIGS. 1A-1E; the genes BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1, and/or XPC; or the genes MDM2, CDKN1A, ZMAT3, DDB2, FDXR, RPS27L, BAX, RRM2B, SESN1, CCNG1, XPC, TNFRSF10B and AEN; or the genes RPS27L, FDXR, CDKN1A and AEN (and optionally MDM2)) in a cancer or tumor sample from the subject relative to a control, e.g., an increase in protein expression level, is predictive or indicative of the subject's sensitivity to an MDM2i, and hence, the subject's potential to respond to MDM2i treatment.

Any conventionally known or standard immunoassay format, e.g., ELISA, Western blot, or RIA assay, can be used to measure protein levels in samples undergoing analysis or testing. Antibodies specific for the proteins encoded by the genes in the gene signatures described herein, e.g., in FIGS. 1A-1E, Table 1, or in the gene set RPS27L, FDXR, CDKN1A and AEN (and optionally MDM2) can be used for detection and quantification of proteins by one of a number of suitable immunoassay methods that are well known in the art, such as, for example, those presented in Harlow and Lane (Antibodies: A Laboratory Manual, Cold Spring Harbor Laboratory, New York, 1988, and later editions thereof). Specific antibodies directed to the proteins encoded by the genes of the disclosed gene signatures can be generated using standard methods known to the skilled practitioner.

Immunohistochemical/staining techniques can also be utilized for gene detection and quantification, for example, using formalin-fixed, paraffin embedded (FFPE) slides, optionally used with an automated slide stainer, e.g., as is available from Ventana Medical Systems, Inc., Tucson, Ariz., as well as other commercial vendors. General guidance for performing these techniques can be found, for example, in Bancroft and Stevens, 1982, Theory and Practice of Histological Techniques, Churchill Livingstone and in Ausubel et al., 1998, Current Protocols in Molecular Biology, John Wiley & Sons, New York, and more recent editions thereof.

Quantitative spectroscopic methods, such as surface-enhanced laser desorption-ionization (SELDI), can be used to analyze protein expression in a cancer or tumor tissue or cell sample, as well as non-cancerous cells or tissue, and cells or tissue from a cancer-free subject. SELDI is a solid phase method for desorption in which the analyte is presented to the energy stream on a surface that enhances analyte capture or desorption In one example, SELDI time-of-flight (SELDI-TOF) mass spectrometry is used to detect protein expression, for example, by using the ProteinChip™ (Ciphergen Biosystems, Palo Alto, Calif.). These types of methods are well known to and practiced by those having skill in the art. For example, see U.S. Pat. Nos. 5,719,060; 6,897,072; and 6,881,586. Alternatively, antibodies are immobilized onto the chromatographic surface using an Fc binding support, or bacterial Fc binding support. Thereafter, the surface is incubated with a sample, such as a cancer sample, and the antibodies on the surface can recognize and bind the antigens present in the sample. Unbound proteins and mass spectrometric interfering compounds are washed away, and the proteins that are bound by antibody and retained on the chromatographic surface are analyzed and detected, such as by SELDI-TOF. The Mass Spectrometry profile from the sample can be compared using differential protein expression mapping, wherein relative expression levels of proteins at specific molecular weights are compared by a variety of statistical techniques and bioinformatic software systems.

In an embodiment, the expression of MDM2i sensitive genes within the gene signatures of the invention can be characterized in a number of cancer or tumor tissue specimens using a tissue microarray. (See, e.g., Kononen et al., 1998, Nature Medicine, 4(7):844-47). In such a tissue array, multiple tissue samples, e.g., from a subject having a cancer, tumor, or neoplasm, can be assessed on the same microarray. The expression levels of RNA and protein are detectable in situ, and multiple samples can be analyzed simultaneously in consecutive sections, if desired.

### Kits and Associated Reagents

The invention provides reagents and kits for practicing one or more of the methods of the invention. The reagents contemplated are those that are specifically designed for use in practicing the methods and utilizing the described gene signatures indicative of MDM2i sensitivity in accordance with the invention. In an example, a reagent is an array of probe nucleic acids in which the gene signature genes of interest are represented. As described herein, a variety of different array formats are known and used in the art and can include a wide variety of different probe structures, substrate compositions and attachment technologies. For guidance without limitation, representative array structures are exemplified in U.S. Pat. Nos. 5,143,854; 5,288,644; 5,324,633; 5,470,710; 5,492,806; 5,503,980; 5,510,270; 5,525,464; 5,547,839; 5,580,732; 5,661,028; 5,800,992; 6,489,159; WO 1996/31622; WO 1997/10365; and WO 1997/27317. In certain embodiments, the number of genes of the MDM2i gene sensitivity signatures as represented on the array is at least 3, at least 4, at least 5, at least 10, at least 25, and can be at least 40, 50, 100, up to and including all of the gene signature genes indicative of MDM2i sensitivity.

Expression profiles of genes within the MDM2i sensitive gene signatures can be generated by employing reagents tailored for inclusion in the kits of the invention. Such reagents comprise a collection of gene specific nucleic acid primers and/or probes designed to selectively detect and/or amplify gene signature genes for use in detecting gene expression levels by using any assay format, e.g., polymerase-based assays (RT-PCR, TAQMAN™), hybridization-based assays, e.g., using DNA microarrays or other solid supports, nucleic acid sequence-based amplification assays, or flap endonuclease-based assays, or other nucleic acid quantification methods. Examples of gene specific primers and methods for their use can be found in U.S. Pat. No. 5,994,076. Of particular interest are reagents comprising collections of gene specific probes and/or primers for at least 3, 4, 5, 8, 10, or all, of the MDM2i sensitive gene signature genes, or for a plurality of these genes, e.g., at least 25, at least 30, 40, 50, 100 or more, up to the inclusion of 177 of the genes in a gene signature, e.g., as set forth in FIGS. 1A-1Es; in Table 1, or the gene signature subset having the genes RPS27L, FDXR, CDKN1A and AEN (and optionally MDM2). The gene specific probe and/or primer collections may include only gene signature genes, or they may include probes and/or primers for additional genes.

Accordingly, the probes and/or primers used in the kits embrace oligonucleotides or antisense nucleic acids that are wholly or partially complementary to the gene biomarkers comprising the gene signatures of the invention, which serve as targets predictive and indicative of the sensitivity of a sample undergoing testing to an MDM2i, particularly in connection with usage of the kits. It is contemplated that the kits will include instructions for practicing the subject methods, and, as applicable, values and parameters, such as sensitivity scores, cutoff values, or control data, to allow interpretation of the results obtained from use of the kits. As noted, the instructions may be provided as printed information on a suitable medium, such as one or more paper documents, in the packaging of the kit, in a package insert, in a label, etc. In addition, instructions may be provided on a computer-readable medium, e.g., a diskette, CD, DVD, tape, etc., on which the information has been recorded. Alternatively, instructions can be provided through a website address which may be accessed and used via the internet and a computer or other suitable device to access the information remotely or off-site.

The kits may also include a software package for statistical analysis of one or more results related to the sensitivity of a sample to MDM2i treatment, and may include a reference database for calculating the probability of sensitivity to the inhibitor. The kit may include reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post-synthesis labeling reagents, such as chemically active derivatives of fluorescent dyes, enzymes, e.g., reverse transcriptases, DNA polymerases, RNA polymerases, and the like, various buffer media, e.g., hybridization and washing buffers, prefabricated probe arrays, labeled probe purification reagents and components, e.g., spin columns, etc., signal generation and detection reagents, e.g., streptavidin-alkaline phosphatase conjugate reagents, chemifluorescent or chemiluminescent substrate reagents, and the like.

In another embodiment, the kits of the invention comprise sets of the MDM2i sensitivity gene biomarkers embraced by the gene signatures described herein. In an embodiment, the kit contains a microarray which is ready for hybridization to target polynucleotide molecules, instructions for use of the components, and/or software for data analysis using computer systems as described below and known by those having skill in the art. In an embodiment, the kit comprises probe arrays containing nucleic acid primers and/or probes for determining the level of expression in a subject's cancer or tumor sample or specimen, or in a cell culture, of a plurality of genes, such as the genes provided in the gene signatures (MDM2i gene sensitivity signature genes of the invention (and/or TP53 status indicator genes). The probe array may contain, for example, 177 probes or fewer, or 100 probes or fewer, or 50 probes or fewer, or 40 probes or fewer, or 3-10 probes (including numbers therebetween) to provide a customized set for identifying gene expression signatures and profiles as described herein. In an embodiment, the kit may contain primers and/or probes for evaluating the sensitivity of a sample to an MDM2i, as well as primers and/or probes for performing necessary or appropriate assay controls, such as expression level controls.

In another embodiment, a kit is provided for carrying out a method of the invention which allows a prediction of the sensitivity of a patient's cancer or tumor to MDM2i treatment, wherein the method comprises a) analyzing, in a sample obtained from the patient, expression levels of at least three genes, at least four genes, at least five genes, at least six genes, at least ten genes, at least twenty genes, at least thirty genes, at least forty genes, or all of the genes within an MDM2i gene sensitivity signature as set forth in FIGS. 1A-1E or Table 1 or in the gene set RPS27L, FDXR, CDKN1A and AEN; and b) comparing the expression levels of the at least three, etc., gene signature genes in the sample to control expression levels; and assigning the cancer or tumor as MDM2i sensitive based on correlation with expression levels observed in previously analyzed patient samples cohorts of known MDM2 sensitivity outcome, thereby predicting the patient's cancer or tumor as being sensitive to the MDM2i.

In another embodiment, a kit is provided for analyzing in a cancer or tumor sample of a patient the protein expression levels of protein products encoded by the at least three, etc., genes of the gene signatures as provided in FIGS. 1A-1E, Table 1, or in the gene signature having the genes RPS27L, FDXR, CDKN1A and AEN (and optionally MDM2), wherein the kit comprises antibodies immunologically specific for the protein products or fragments thereof, means for detecting immune complex formation between the protein products and the antibodies and instructional materials comprising ranges of expression levels associated with MDM2i sensitivity of the sample.

In another embodiment, a kit is provided for analyzing in a patient's cancer or tumor sample the expression levels of at least three, at least four, at least five, etc., or all, of the nucleic acids or information sequence fragments thereof corresponding to genes within the gene signatures as provided in FIGS. 1A-1E, Table 1, or in the gene signature having the genes RPS27L, FDXR, CDKN1A and AEN (and optionally MDM2), in which the kit comprises nucleic acids which specifically hybridize to the nucleic acids of the gene signature genes; means for detecting hybridization between the hybridizing nucleic acids; and instructional materials including ranges of expression levels or cutoff values associated with MDM2 sensitivity of the sample.

In another embodiment, the invention provides a kit for assessing a patient's cancer or tumor sensitivity to an MDM2i in which the assessment is made with a test apparatus, the kit comprising reagents for collecting a test sample from a patient; and reagents for measuring the expression of at least three, at least four, or all, of the genes in a gene signature of the invention, such as the genes in FIGS. 1A-1E; in Table 1; or in the gene signature having the RPS27L, FDXR, CDKN1A and AEN (and optionally MDM2) genes, or variants thereof, in a patient's test sample and packaging and instructions therefor. In an embodiment related to the kit, the reagents for collecting a test sample are reagents for collecting a blood or tissue sample. In an embodiment related to the kit, the reagents for measuring the expression profile of a gene signature are reagents for real-time polymerase chain reaction (RT-PCR), quantitative RT-PCR, an array or microarray, or an immunochemical assay or specific oligonucleotide hybridization.

### Pharmaceutical Composition

In an aspect, the invention provides a pharmaceutical composition for use in treating a cancer or tumor in a subject, wherein the composition comprises at least one MDM2i as defined above, and wherein the subject has been determined as sensitive to the MDM2i treatment by assessing the sensitivity of a subject's cancer or tumor to the MDM2i treatment by any of the above-described methods for predicting the sensitivity.

In an embodiment, the pharmaceutical composition may be used in treating melanomas.

The method which can be used to select the subject to be administered the present pharmaceutical composition is a method for predicting the sensitivity of a subject's cancer or tumor to MDM2i treatment, comprising: a) measuring the levels of expression of at least three genes selected from the genes listed in FIGS. 1A-1E in a cancer or tumor sample obtained from the subject; b) scoring the levels of expression of the at least three genes to obtain a subject's sensitivity score; c) measuring the levels of expression of the at least three genes in plurality of cancers or tumors sample whose sensitivities to MDM2i treatment are unknown; d) scoring the levels of expression of the at least three genes to obtain a reference score in each sample and determining a threshold based on the distribution of the reference scores; and e) predicting that the subject is sensitive to MDM2i treatment if the subject's sensitivity score is over the threshold and the subject is resistant to MDM2i treatment if the subject's sensitivity score is under the threshold.

In a particular embodiment, step e) is predicting that the subject is sensitive to MDM2i treatment if the subject that has been predicted as resistant has amplified MDM2 genes in its genome. In an embodiment, steps b) and d) comprise summing the normalized scores of the levels of the gene expression. In an embodiment, the threshold is determined based on Receiver Operating Characteristic (ROC) plots optionally by conducting leave-one-out cross-validation (LOOCV) analysis. In an embodiment, the threshold is determined from the shape of the distribution of the reference scores, for example, by binalization algorithms such as Otsu's method. In an embodiment, the threshold is determined by Gaussian Mixture model as described above.

In the pharmaceutical composition of the invention, MDM2i can be selected from a group consisting of Compound A and salts thereof, Compound B and salts thereof, CGM097, RG7388, MK-8242 (SCH900242), MI-219, MI-319, MI-773, MI-888, Nutlin-3a, RG7112 (R05045337), TDP521252, TDP665759, PXN727, PXN822, and a combination thereof as described herein.

### Computer Facilitated Analysis

In certain embodiments, practice of the invention in one or more of its aspects may involve the use of a computer and its related systems and components. Such a computer system and components as referred to herein signify, without limitation, the hardware, software and data storage means used to analyze and evaluate information from certain embodiments of the invention. In some embodiments, the computer systems include a central processing unit (CPU), as well as input means, output means, and data storage means. Any one, or several, of the currently available computer-based systems are suitable for use in accordance with the invention, as will be appreciated by the skilled practitioner. The data storage means may include any means or device comprising a recording of data and information generated from the methods of the invention, or a memory access means that can access such a means or device. Such description of relevant computer-related information as applicable to the invention can be found, for example, in WO 2013/071247.

Any of the comparison steps involved in the analytic methods associated with aspects of the described invention may be performed by means of software components loaded or programmed into a computer or other (electronic) information machine, or digital device. With the appropriate components, data, and included information, the computer, machine, or device may then perform the required steps to assist the analysis of values associated with one or more genes (for example, a value that correlates with the expression of a particular gene in the manner described above, or for comparing such associated values). The features embodied in one or more computer programs may be performed by one or more computers running such programs. In some embodiments, a computer system suitable for implementation of the analytic methods related to the invention includes internal components, which include a processor element interconnected with a main memory. The computer system is further linked to external components, including mass storage (e.g., one or more hard disks typically packaged together with the processor and memory and having variable storage capacity); user interface devices (e.g., a monitor), together with an inputting device, which can be a “mouse”, or other graphic input devices, and/or a keyboard). A printer or printing device can also be attached to the computer. Typically, the computer system is also linked to a shared network link, which can be part of an Ethernet link to other local computer systems, remote computer systems, or to wide area communication networks, such as the Internet, such as is also described in WO 2013/071247.

For its operation, the system typically has loaded into its memory several software components, which are both standard in the art and special to the MDM2i sensitivity gene signatures described herein. These software components collectively cause the computer system to function according to the disclosed methods. In some embodiments, the software components are stored on mass storage. In some embodiments, the software components include an operating system (OS), which is responsible for managing the computer system and its network interconnections. For example, the OS can be the Microsoft Windows family, e.g., Windows 7, or earlier or later versions, or those of other providers, including Apple, for example. In addition, the software components include common languages and functions conveniently present on the system to assist programs implementing the disclosed methods. Several high or low level computer languages can be used to program the analytic methods. Instructions can be interpreted during run-time or compiled. Exemplary computer languages include, without limitation, C/C++, FORTRAN and Rand JAVA®. In an embodiment, the methods are programmed in mathematical software packages that allow symbolic entry of equations and high-level specification of processing, including algorithms to be used, thereby alleviating user programming of individual equations or algorithms. Such packages include, without limitation, Matlab from Mathworks (Natick, Mass.), Mathematica from Wolfram Research (Champaign, Ill.), and S-Plus from Math Soft (Cambridge, Mass.).

As an example of implementation for the practice of the methods, a user, e.g., a clinician, medical or healthcare technician, practitioner, information specialist, or combination thereof, as a first step, loads microarray experiment data into the computer system. These data can be directly entered by the user or from other computer systems linked by the network connection, or on portable, removable storage media such as a CD-ROM, data storage device (e.g., USB flash drive), tape drive, ZIP® drive or through the network. The user then causes execution of expression profile analysis software, which performs the disclosed methods. Another exemplary implementation involves a user who loads microarray experiment data into the computer system. This data is loaded into the memory from the storage media or from a remote computer, such as from a dynamic geneset database system, through the network. Next the user executes the software that performs the comparison of gene expression data from a cancer sample with a control (as described herein) to detect a difference of gene expression between the cancer sample and the control. Alternative computer systems and software for implementing the analytic methods associated with the invention will be known and apparent to one skilled in the art.

Accordingly, any of the described methods can be implemented as computer-executable instructions stored on one or more computer-readable storage media (e.g., non-transitory computer-readable media, such as one or more optical media discs, volatile memory components (such as DRAM or SRAM), or nonvolatile memory components (such as hard drives) and executed on a computer (e.g., any commercially-available computer, including smart phones, iPads and the like, or other mobile devices that include computing hardware). Any of the computer-executable instructions for implementing the disclosed techniques, as well as any data created and used during implementation of the described methods and embodiments, can be stored on one or more computer-readable media (e.g., non-transitory computer-readable media). The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network, such as a cloud computing network, or other such network) using one or more network computers. As will be appreciated by the skilled practitioner in the art, only certain selected aspects of the software-based implementations are described. Any details that are not described herein are well known and/or conventional to the skilled practitioner in the art. Further, the technology as related to aspects of the invention is not limited to any particular computer or hardware type. Specific details of suitable computers, hardware and related components are well known and are not set forth in detail herein, in view of the general knowledge possessed by those skilled in the art.

In addition, any of the software-based aspects, including, for example, computer-executable instructions for causing a computer to perform any of the disclosed methods, can be uploaded, downloaded, or remotely accessed through a suitable means of communication, including, without limitation, the Internet, the World Wide Web, the Cloud, an intranet, software applications, cable (including fiber optic cable), magnetic communications, electromagnetic communications (including RF, microwave and infrared communications), electronic communications, etc. Furthermore, any of the computer-readable media of use herein can be non-transitory (e.g., memory, magnetic storage, optical storage, or the like). Any of the storing actions of use with the methods can be implemented by storing in one or more computer-readable media (e.g., computer-readable storage media or other tangible media). Anything stored can be in one or more computer-readable media (e.g., computer-readable storage media or other tangible media) such that the methods and systems described herein can be implemented by computer-executable instructions in (e.g., encoded on) one or more computer-readable and/or portable media (e.g., computer-readable storage media, storage devices, or other tangible media). As such, the instructions can cause a computer to perform the method, and the technologies described herein can be implemented in a variety of programming languages.

Some embodiments of the invention may include a method performed, at least in part, by a computer system, the computer system including a screen, software that displays gene expression levels on the screen, a keyboard and/or mouse for interfacing with the software, and a memory that stores a list or lists of the expression levels of genes in a cancer sample or specimen undergoing testing, evaluation, or analysis for MDM2i sensitivity. The method includes, for example, analyzing in the list or lists of genes associated with an MDM2i gene sensitivity signature, the level of expression in a cancer sample or specimen of, for example, three or more, four or more, or five or more, or six or more, etc., or all, of the genes, of a gene signature of the invention, e.g., the genes listed in FIGS. 1A-1E; in Table 1; or in the gene signature having the genes RPS27L, FDXR, CDKN1A, and AEN (and optionally MDM2), comparing to a control level of expression data set of the same numbers of genes; and identifying the cancer (or tumor or neoplasm) as sensitive to treatment with MDM2i treatment when an increase in the level of expression of the specified number of genes in the cancer, tumor, or neoplasm sample relative to the control exceeds a predefined limit, or can be related to a sensitivity score or cutoff value. As but one, nonlimiting example, the predefined limit (i.e., a cutoff value) can be 0.2. In this case, a value of >0.2 is considered a high score or cutoff value and signifies high sensitivity to an MDM2i, while a value of <0.2 is considered a low score or cutoff value and signifies low sensitivity to the MDM2i.

In an embodiment, the invention provides a method comprising implementation, at least in part by a computer, in which a gene expression dataset (e.g., a list of gene expression levels) comprising a gene expression level for each of the gene signature genes of FIGS. 1A-1E, Table 1, or of the gene signature having the genes RPS27L, FDXR, CDKN1A, and AEN (and optionally MDM2) is received. The expression levels of the genes in the dataset are compared to control gene expression levels of the same genes, and a difference in the gene expression level of the genes in the dataset compared with the control gene expression level of the same genes is calculated. In some embodiments, the calculated difference in the gene expression level of the genes in the dataset compared to the control gene expression level of the same genes, or normalized to control (housekeeping) gene expression levels, is displayed in a user interface. In other embodiments, the method further comprises identifying the cancer, tumor, or neoplasm (or sample thereof) as sensitive to treatment with MDM2i, if there is a difference in the expression levels of the genes in the dataset as compared to the control expression levels of the same genes, or to the normalized value, for example, if the sensitivity score or cutoff value of the expression of genes in the dataset is above a threshold or cutoff value that is indicative of sensitivity of the cancer, tumor, or neoplasm to an MDM2i. In further embodiments, one or more computer-readable storage devices comprising computer-executable instructions for performing any one or more of the methods described herein are provided.

In an embodiment, the invention provides a computer program product for determining whether a subject's cancer or tumor is sensitive to treatment with an MDM2i, wherein the computer program product, when loaded onto a computer, is configured to employ a gene expression result from a cancer or tumor sample derived from a subject to determine whether the subject's cancer or tumor is MDM2i sensitive, wherein said gene expression result comprises expression data for all or a subset of (e.g., 3, 4, 5, 6, 8, 10, or more) genes of the gene signatures listed in FIGS. 1A-1E; in Table 1; or in the gene signature containing the genes RPS27L, FDXR, CDKN1A, and AEN, or as otherwise provided by the invention.

### EXAMPLES

The following examples are provided to illustrate particular features and/or embodiments of the invention. The illustrated features and/or embodiments serve to exemplify the invention and are not intended to be limiting.

### Example 1

This Example describes an evaluation of the effect of a representative small molecule MDM2i on the growth of cells in a multi-cancer cell line panel. In this Example, the MDM2 inhibitors used were Compound A and Compound B p-toluenensulfonate. The panel included 250 human cancer cell lines (OncoPanel™, Ricerca Biosciences, Painesville, Ohio) that were evaluated in a high content drug screening analysis. The relative IC₅₀ values for the cell lines were determined.

Materials and Methods

Compounds were weighed using an electronic balance (AX205, Serial No. 1126051685, Mettler-Toledo K.K.) and was provided to Ricerca Biosciences for testing using its panel of cancer cell lines in its commercial OncoPanel cytotoxicity assay.

Oncopanel™ Cytotoxicity Assay

Cells were grown in RPMI 1640, 10% FBS, 2 mM L-alanyl-L-Glutamine, 1 mM Na Pyruvate, or a special medium in a humidified atmosphere of 5% CO2 at 37° C. Cells were seeded into 384-well plates and incubated in a humidified atmosphere of 5% CO2 at 37° C. Test compounds were added 24 hours post cell seeding. At the same time, a time zero, untreated cell plate was generated as a control. After a 72 hour incubation period, cells were fixed and stained with nuclear dye to allow visualization of nuclei.

Compounds were serially diluted 3.16-fold and assayed over 10 concentrations of inhibitor in a final assay concentration of 0.1% DMSO from the highest test concentration specified in the sample information chapter. Automated fluorescence microscopy was carried out using a GE Healthcare InCell Analyzer 1000, and images were collected with a 4×objective. Twelve bit tiff images were acquired using the InCell Analyzer 1000 3.2 and analyzed with Developer Toolbox 1.6 software.

Cell proliferation was measured by the signal intensity of the incorporated nuclear dye. The cell proliferation assay output is referred to as the relative cell count. To determine the cell proliferation end point, the cell proliferation data output is transformed to percent of control (POC) using the following formula:

POC=relative cell count(compound wells)/relative cell count(vehicle wells)×100.

Relative cell count IC₅₀ (IC₅₀) is the test compound's concentration that produces 50% of the cell proliferation inhibitory response or 50% cytotoxicity level. IC₅₀ values were calculated using nonlinear regression to fit data to a sigmoidal 4 point, 4 parameter One-Site dose response model, where: y (fit)=A+[(B−A)/(1+((C/x)̂D))]. Curve-fitting, IC₅₀ calculations and report generation were performed using a custom data reduction engine MathIQ based software (AIM). In addition, IC₅₀ values were not calculated in cell lines in which Compound A did not reduce the growth of treated cells to half that of untreated cells at the highest concentration of 40.0 μM, and Compound B p-toluenensulfonate did not reduce the growth of treated cells to half that of untreated cells at the highest concentration of 10.0 μM. In these cases, the IC₅₀ values were expressed as 40 μM and 10 μM, respectively (Table 2).

TABLE 2

| Name of | Compound A | Compound B |
|---|---|---|
| Cell Line | IC₅₀ (μM) | IC₅₀ (μM) |
| 22Rv1 | 1.31 | 0.109 |
| 5637 | 40 | 10 |
| 639-V | 40 | 6.39 |
| 647-V | 40 | 10 |
| 769-P | 0.442 | 0.036 |
| A-673 | 40 | 9.21 |
| A101D | 0.233 | 0.0314 |
| A172 | 0.795 | 0.0689 |
| A204 | 0.755 | 0.0294 |
| A375 | 0.317 | 0.0434 |
| A427 | 1.65 | 0.143 |
| A431 | 40 | 10 |
| A498 | 1.71 | 0.119 |
| A549 | 0.401 | 0.0769 |
| ACHN | 1.068 | 0.174 |
| AGS | 0.139 | 0.0116 |
| AN3 CA | 40 | 8.75 |
| ARH-77 | 40 | 7.61 |
| AU565 | 23.7 | 10 |
| AsPC-1 | 40 | 10 |
| BC-1 | 0.464 | 0.074 |
| BFTC-905 | 40 | 8.51 |
| BHT-101 | 40 | 10 |
| BPH1 | 40 | 10 |
| BT-549 | 40 | 10 |
| BT20 | 40 | 10 |
| BT474 | 40 | 10 |
| BV-173 | 0.601 | 0.0873 |
| BxPC-3 | 40 | 10 |
| C-33A | 40 | 5.49 |
| C-4 II | 40 | 9.1 |
| C32 | 40 | 10 |
| CAL-62 | 40 | 10 |
| CAMA-1 | 40 | 10 |
| CCF-STTG1 | 0.541 | 0.0852 |
| CCRFCEM | 40 | 10 |
| CFPAC-1 | 40 | 10 |
| CGTH-W-1 | 40 | 8.04 |
| CHL-1 | 40 | 9.1 |
| CHP-212 | 0.196 | 0.0294 |
| CML-T1 | 2.38 | 0.0597 |
| COLO 829 | 0.289 | 0.0266 |
| CRO-AP2 | 0.204 | 0.0191 |
| CaOV3 | 40 | 10 |
| Caki-1 | 0.275 | 0.0387 |
| Cal 27 | 40 | 10 |
| Calu1 | 40 | 10 |
| Calu6 | 40 | 5.38 |
| Capan-1 | 40 | 10 |
| Capan-2 | 40 | 10 |
| ChaGoK1 | 40 | 10 |
| Colo 205 | 36.7 | 9.48 |
| Colo 320 HSR | 40 | 10 |
| D283 Med | 0.529 | 0.0819 |
| DB | 40 | 5.98 |
| DBTRG-05MG | 0.132 | 0.0846 |
| DK-MG | 0.645 | 0.0553 |
| DMS114 | 40 | 10 |
| DMS53 | 38.4 | 10 |
| DOHH-2 | 0.123 | 0.0288 |
| DU145 | 40 | 7.91 |
| Daoy | 40 | 10 |
| Daudi | 0.599 | 0.132 |
| Detroit 562 | 40 | 7.74 |
| DoTc2 4510 | 39.6 | 10 |
| EB-3 | 40 | 10 |
| EFM-19 | 40 | 10 |
| EM-2 | 40 | 10 |
| FaDu | 40 | 10 |
| G-401 | 0.242 | 0.0354 |
| G-402 | 0.568 | 0.0261 |
| H4 | 0.172 | 0.024 |
| HCT-116 | 0.653 | 0.0505 |
| HCT-15 | 32 | 10 |
| HLE | 28.9 | 10 |
| HOS | 40 | 10 |
| HPAF-II | 40 | 5.79 |
| HT | 40 | 10 |
| HT-1080 | 0.282 | 0.062 |
| HT-1197 | 0.235 | 0.135 |
| HT-29 | 33.5 | 5.14 |
| HT-3 | 40 | 10 |
| HT1376 | 38.9 | 10 |
| HUH-6 Clone 5 | 0.329 | 0.0719 |
| Hs 578T | 40 | 10 |
| HuCCT1 | 40 | 10 |
| HuP-T4 | 40 | 10 |
| J-RT3-T3-5 | 40 | 10 |
| J82 | 40 | 10 |
| JAR | 0.549 | 0.0861 |
| JEG-3 | 3.62 | 0.249 |
| K562 | 40 | 10 |
| KATO III | 36.6 | 10 |
| KLE | 40 | 10 |
| L-428 | 40 | 10 |
| LS-174T | 0.497 | 0.0615 |
| LS1034 | 40 | 10 |
| MALME3M | 1.04 | 0.0875 |
| MC-IXC | 22.7 | 8.62 |
| MCF7 | 0.661 | 0.0833 |
| MDA MB 231 | 40 | 10 |
| MDA MB 453 | 32.8 | 9.29 |
| MDA MB 468 | 36.2 | 8.12 |
| MEG01 | 40 | 9.7 |
| MES-SA | 0.529 | 0.0641 |
| MG-63 | 40 | 10 |
| MHH-PREB-1 | 40 | 8.3 |
| MOLT-16 | 0.236 | 0.0598 |
| MV-4-11 | 0.372 | 0.0254 |
| MeWo | 40 | 10 |
| Mia PaCa-2 | 40 | 7.22 |
| NALM-6 | 0.658 | 0.065 |
| NCI-H292 | 0.479 | 0.0347 |
| NCI-H460 | 0.515 | 0.0664 |
| NCI-H508 | 40 | 10 |
| NCI-H520 | 40 | 10 |
| NCI-H596 | 40 | 10 |
| NCI-H661 | 40 | 6.59 |
| NCI-H747 | 40 | 10 |
| NCIH441 | 40 | 10 |
| NCIH446 | 40 | 10 |
| OVCAR3 | 40 | 10 |
| PC-3 | 40 | 10 |
| RD | 40 | 7.2 |
| RKO | 0.654 | 0.107 |
| RL95-2 | 13.8 | 3.72 |
| RPMI 6666 | 1.7 | 0.0793 |
| RPMI 8226 | 40 | 0.065 |
| RPMI-7951 | 40 | 8.63 |
| Raji | 13.8 | 5.32 |
| SCC-25 | 40 | 9.86 |
| SCC-4 | 40 | 10 |
| SCC-9 | 40 | 10 |
| SH-4 | 0.59 | 0.116 |
| SHP-77 | 40 | 8.32 |
| SJSA1 | 0.214 | 0.0261 |
| SK-LMS-1 | 40 | 10 |
| SK-MEL-1 | 0.207 | 0.0293 |
| SK-MEL-28 | 40 | 10 |
| SK-MEL-3 | 40 | 10 |
| SK-N-AS | 40 | 8.29 |
| SK-N-DZ | 40 | 10 |
| SK-N-FI | 40 | 10 |
| SK-NEP-1 | 40 | 10 |
| SK-UT-1 | 40 | 10 |
| SKMES1 | 33.8 | 8.73 |
| SKOV3 | 40 | 10 |
| SNB-19 | 40 | 10 |
| SNU-423 | 40 | 10 |
| SR | 0.257 | 0.0262 |
| ST486 | 24.3 | 6.44 |
| SW-13 | 8.33 | 8.45 |
| SW1088 | 40 | 9.02 |
| SW1116 | 40 | 10 |
| SW1417 | 40 | 10 |
| SW1463 | 40 | 10 |
| SW1783 | 40 | 10 |
| SW48 | 1.09 | 0.125 |
| SW620 | 40 | 7.12 |
| SW684 | 40 | 10 |
| SW837 | 40 | 10 |
| SW872 | 40 | 10 |
| SW900 | 40 | 10 |
| SW948 | 40 | 10 |
| SW954 | 40 | 8.33 |
| SW962 | 40 | 10 |
| SW982 | 0.232 | 0.0209 |
| SaOS2 | 40 | 10 |
| SiHa | 40 | 10 |
| T24 | 40 | 10 |
| T47D | 32.3 | 7.76 |
| T98G | 40 | 10 |
| TCCSUP | 40 | 10 |
| Thp1 | 40 | 10 |
| U-87 MG | 0.261 | 0.0376 |
| U2OS | 1.24 | 0.248 |
| UM-UC-3 | 40 | 10 |
| YAPC | 40 | 10 |
| 786-O | 37.3 | 0.342 |
| A7 | 40 | 3.59 |
| BE(2)C | 35.3 | 9.03 |
| BM-1604 | 40 | 10 |
| BeWo | 1.49 | 0.317 |
| C-4 I | 40 | 7.21 |
| C32TG | 0.36 | 0.0329 |
| CEM-C1 | 33.6 | 9.37 |
| Caki-2 | 0.472 | 0.0821 |
| Colo 201 | 34.6 | 7.33 |
| Colo 320DM | 40 | 10 |
| DLD-1 | 40 | 8.91 |
| ES-2 | 14.5 | 6.3 |
| HCT-8 | 0.506 | 0.0516 |
| HEC-1-A | 40 | 10 |
| HEL-92-1-7 | 40 | 10 |
| HLF | 40 | 10 |
| HMCB | 40 | 5.49 |
| HS 746T | 38.9 | 5.85 |
| HeLa | 40 | 10 |
| HepG2 | 0.335 | 0.0584 |
| Hs 294T | 1.23 | 3.24 |
| Hs 695T | 1.28 | 0.174 |
| Hs 766T | 40 | 10 |
| KHOS-240S | 40 | 10 |
| KPL-1 | 0.455 | 0.031 |
| MDA-MB-436 | 40 | 10 |
| MOLT-3 | 0.605 | 0.0332 |
| MT-3 | 1.13 | 0.164 |
| NCI-H295R | 40 | 10 |
| OCUG-1 | 40 | 10 |
| PANC-1 | 40 | 6.73 |
| RKO-AS45-1 | 0.471 | 0.074 |
| RKOE6 | 38.2 | 7.59 |
| Ramos (RA 1) | 40 | 8.66 |
| SCaBER | 40 | 10 |
| SK-BR-3 | 27.7 | 10 |
| SKO-007 | 40 | 8.64 |
| SNU-1 | 0.412 | 0.135 |
| SNU-16 | 28.5 | 9.92 |
| SNU-5 | 40 | 10 |
| SU.86.86 | 40 | 8.86 |
| SW1353 | 1.68 | 0.0853 |
| SW403 | 40 | 10 |
| SW480 | 40 | 10 |
| SW579 | 40 | 9.61 |
| TE 381.T | 37.3 | 9.73 |
| U-138MG | 40 | 10 |
| U266B1 | 40 | 10 |
| Wi38 | 0.171 | 0.0379 |
| WiDr | 34.8 | 5.58 |
| Y79 | 1.05 | 0.159 |
| COR-L105 | ND | 0.168 |
| COR-L23 | ND | 8.98 |
| DMS273 | ND | 9.08 |
| NCI-H69 | ND | 10 |
| OE19 | ND | 10 |
| OE33 | ND | 7.58 |
| OE21 | ND | 9.81 |
| SJRH30 | 40 | 7.24 |
| Jurkat | 40 | 10 |
| LNCaP | 0.2704 | 0.064 |
| MX1 | 39 | 9.16 |
| BT-483 | 13.2 | ND |
| CAL-54 | 0.602 | ND |
| CRO-AP5 | 0.22 | ND |
| IMR-32 | 0.828 | ND |
| MDA-MB-175-VII | 40 | ND |
| T84 | 40 | ND |
| VCaP | 40 | ND |
| WM-115 | 0.564 | ND |
| ZR-75-1 | 1.5 | ND |

### Example 2

### Determination of Gene Expression and Gene Signature Information Related to MDM2i Sensitivity

Biomarker Discovery

The cell line response/sensitivity data for the MDM2i inhibitor Compound A was sent to Compendia Bioscience Inc., (Ann Arbor, Mich.) for analysis. The IC₅₀ endpoint values derived from the Oncopanel® data were used to designate cell lines as sensitive or insensitive to the MDM2i. A 1-log difference in IC₅₀ value was maintained between cells designated as sensitive or insensitive to MDM2i. As presented in Example 1, IC₅₀ endpoint values are shown in Table 2. The cell lines were characterized for gene mutations, gene amplification or deletion, and gene over-expression. In addition, pathways of genes with related function were annotated and included as a separate biomarker type. Gene mutation data were available for 186 cell lines present in the 240 multi-cancer cell line panel. Gene mutations were curated from the Wellcome Trust Sanger Institute Cancer Cell Line Project, which determined the sequence of the coding exons and immediate flanking intron sequences of 61 selected cancer-related genes in hundreds of cell lines. The gene mutation data were extracted from the publicly available Sanger database v48 (http://www.sanger.ac.uk/genetics/CGP/CellLines). DNA copy number data and gene expression data were available for all cell lines.

Single nucleotide polymorphism data were converted to DNA copy number estimates using the following method. Cell intensity files from Affymetrix 500K arrays were processed using CRMAv2 in the aroma-affymetrix R package. Chip intensity values were divided by the median reporter values across all cell lines and then log transformed to yield log 2 copy number ratios. Log 2 copy number ratios were processed using circular binary segmentation from the DNACopy package from R/Bioconductor. Segment coordinates intersected with gene coordinates derived from UCSC RefSeq Gene hg18 coordinates were used to generate gene-level copy numbers. In each cell line, genes with log 2 copy number ratios >1 were annotated as amplified and genes with log 2 copy number ratios <−1 were annotated as deleted.

Gene expression data were processed using the GC Robust Multi-array Average (GCRMA) background adjustment algorithm. Alternative chip definition files (altCDF) were used to summarize probes into probe sets associated with Entrez Gene identifiers. The HG-U133 Plus 2.0 Hs_ENTREZG alternative CDF (version 12.1.0) was available from the BrainArray website (http://brainarray.mbni.med.umich.edu/Brainarray/Database/CustomCDF/genomic_curated_CDF.asp). In this CDF, each probe set corresponds to a named gene in the Entrez Gene database. Applying altCDF to the Human Genome U133 Plus 2.0 Arrays chip resulted in the measurement of 17,545 genes. The median expression level of every gene was determined. In each cell line, genes with ≧64-fold expression above the median value were annotated as “Gene Overex.”

Differential expression analysis of the mRNA datasets derived from MDM2i sensitive (n=62) and insensitive (n=164) cell lines was performed to generate custom gene drug sensitivity signatures. A one-tailed Student's t-test was performed to calculate p-values for each gene's differential up-regulation within either sensitive or resistant cell lines. Genes were ranked by p-value and the custom gene signatures for MDM2i sensitivity and insensitivity were each arbitrarily limited to the top 1% of ranked genes (n=177), (e.g., FIGS. 1A-1E).

The potential significance of the association between biomarkers and MDM2i response was characterized using the Fisher's Exact Test, with a null hypothesis of no association between the biomarker (positive or negative) and drug response (sensitivity or resistance). Association tests were computed for all candidate genomic biomarkers called positive in ≧2 cell lines (n=6,996) in sensitive (n=62) and/or resistant (n=164) cell lines. Biomarkers that associated with drug response were initially ranked by p-value and odds ratio. Q-values were calculated as a measure of the false discovery rate due to the large number of association tests performed. Q-values were calculated as (p-value/p-value rank)*number of biomarkers measured, within each biomarker type.

Clinical Population Analysis

Significant in vitro genetic aberration biomarkers were mapped to biomarkers characterized across 20,000+ clinical tumor samples employing concepts analysis. Significant associations of gene signatures were identified with specific cancer subpopulations. More specifically, biomarker profiles associated with in vitro drug sensitivity or resistance were interrogated across clinical genomic data. The Oncomine Integrated Gene Browser and Mutation Browser Power Tools were used to capture frequencies of mutation, over-expression and amplification of selected biomarker genes across cancer types. The Power Tools incorporated data from Oncomine and COSMIC. Mutation frequencies were determined by the number of samples containing mutation/samples measured. Over-expression frequency was determined by the number of samples with expression values of ≧10× the median expression/number of samples. Amplification frequency was determined by the number of samples with estimated copy number values ≧4/number of samples.

Fisher's Exact Test was used to calculate the association of the custom gene signatures with each of the >13,000 existing gene signatures derived from clinical specimens in Oncomine. The null set in calculation was the list of all Entrez genes in the Oncomine database. P-values were calculated in Oncomine, with a null hypothesis of no association between the gene lists. The Q-value was determined as the (p-value/p-value rank)*number of gene lists in Oncomine.

Sensitivity and resistance signatures were scored in patient samples within individual Oncomine datasets and signature expression was characterized in patient subsets defined by metadata such as cancer subtype, molecular subtype, histological grade, or patient outcome. To characterize relative signature expression across cancer types, a custom dataset was created that combined all Oncomine datasets measured on Affymetrix U133 microarray format platforms (>15,000 patient tumor samples). To normalize gene expression distributions across samples from different datasets, the gene expression data were quantile normalized. To normalize the contribution of each gene in the signature, signature genes were subject to Z-score normalization (the mean expression value of each gene is subtracted from the value within each sample and the difference is divided by the standard deviation). Signature scores across the quantile-normalized dataset were generated by determining the unweighted average of the Z-score normalized expression values of each signature gene. Signature scores were summarized by cancer type and cancer subtype. Thresholds for sensitivity and resistance scores derived from in vitro associations with drug response were applied to clinical tumor data to generate frequencies of signature expression across cancer types and subtypes.

Selective Response to the MDM2i Compound A

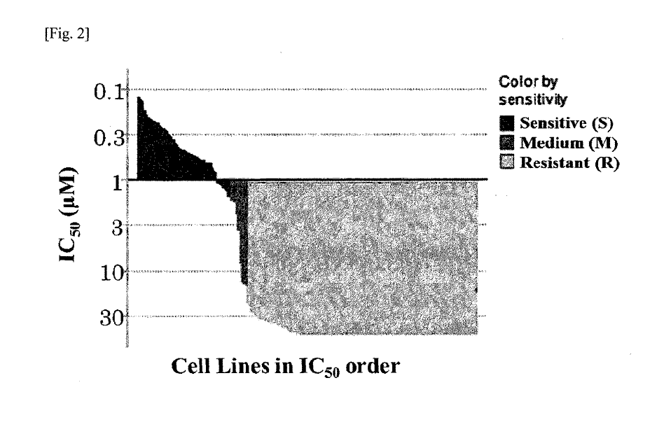

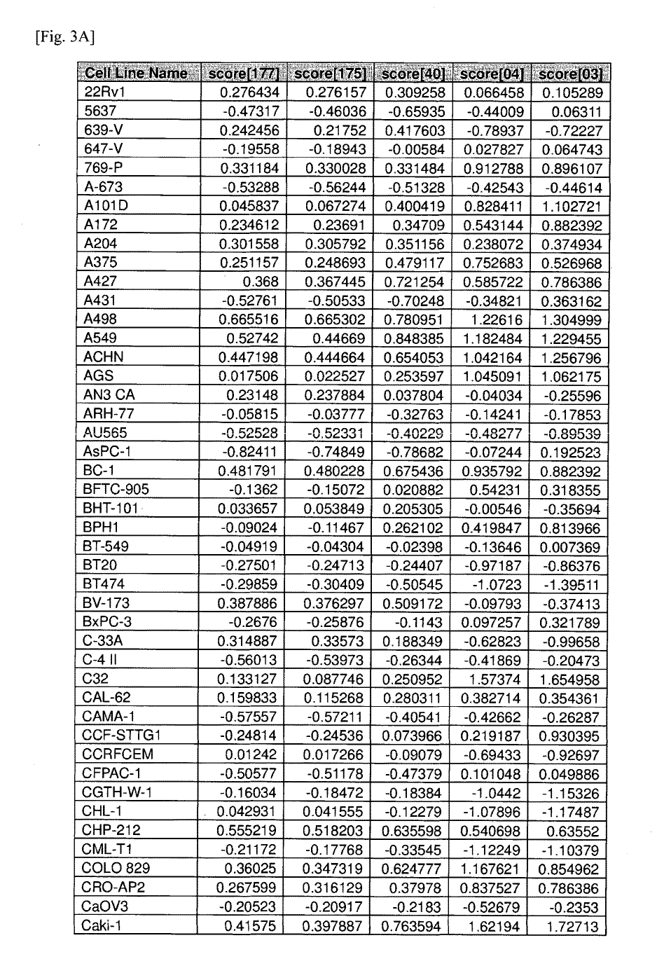

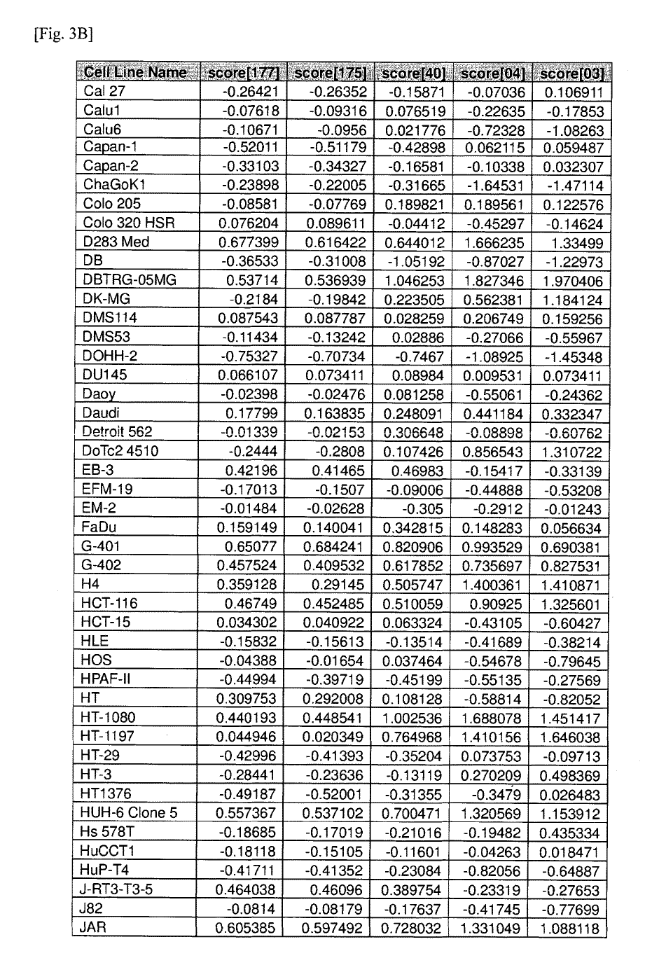

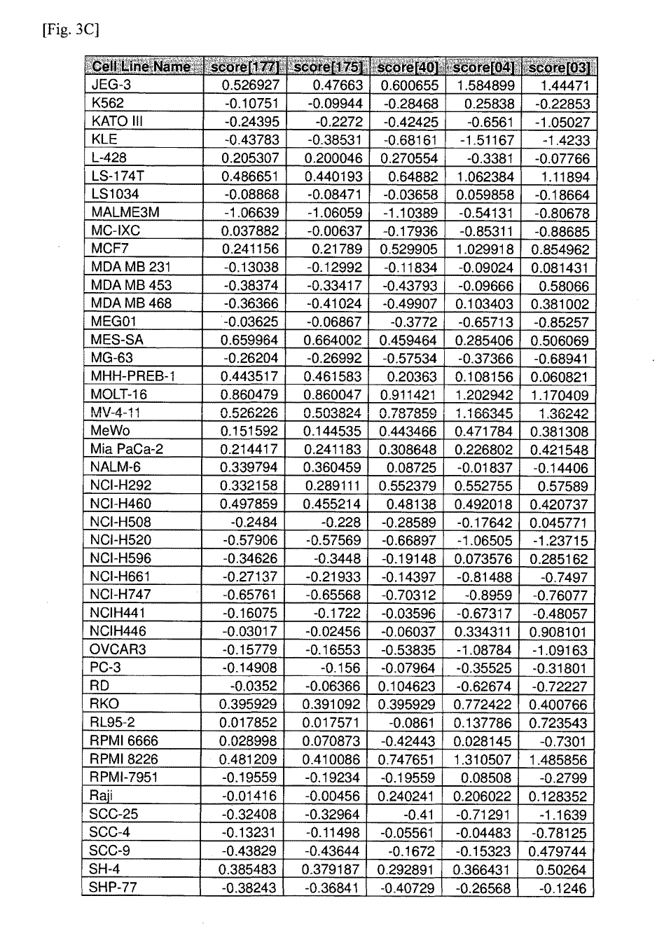

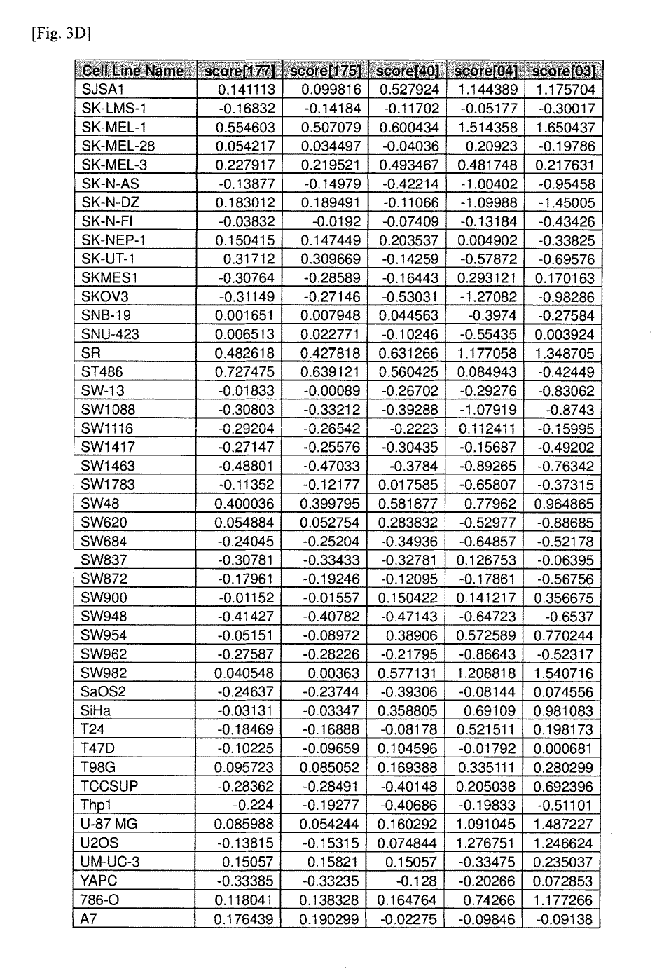

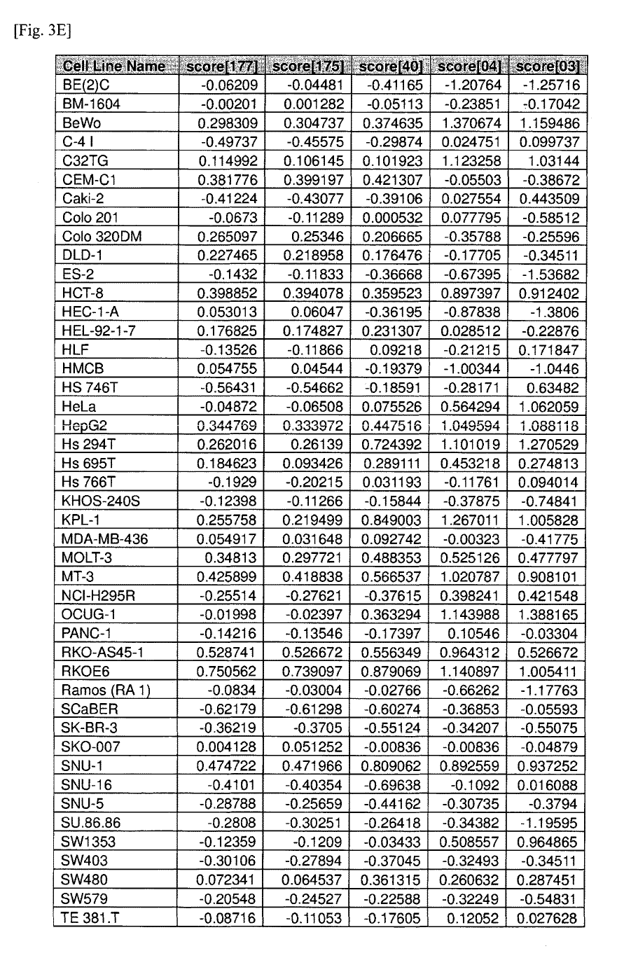

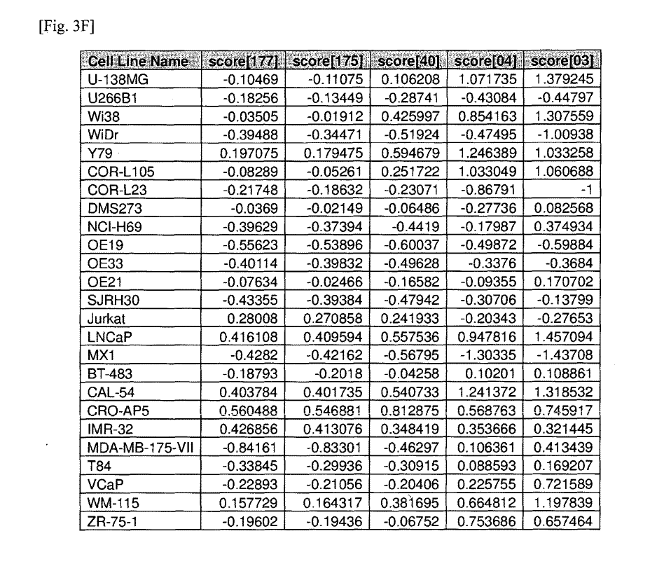

Cell lines were ranked by IC₅₀ value and designated as sensitive (S), moderate (M) and resistant (R) to the MDM2i. The characterization of cell line response by IC₅₀ value indicated two general response phenotypes. Approximately 25% of the cell lines had IC₅₀ values of <1 μM, and approximately 70% had IC₅₀ values of >10 Cell line IC₅₀ values ranged from 0.12 to >50 μM. Cell lines were designated as sensitive or insensitive based on a custom binning strategy that centered a moderate bin of 1 log IC₅₀ over the region of steepest slope on the IC₅₀ waterfall plot. Sixty-two cell lines with lower IC₅₀ values relative to the moderate bin were designated as sensitive and 164 cell lines with IC₅₀ values greater than the moderate bin were designated as resistant. (FIG. 2). Association analyses were performed between the sensitive (n=62) and resistant (n=164) cell line designations and nearly 7,000 candidate genomic biomarkers (classified as positive or negative). TP53 gene mutation strongly associated with resistance to the Compound A MDM2i (p 3E-24, Q 8E-23) and was highly sensitive (0.87) and nearly perfectly specific (0.94).

Multi-Variate Decision Tree Analysis

Partitioning via a single-tree recursive classification algorithm (Accelyrs) or Spotfire (Decision Tree Analysis in Spotfire Decision Site) was used to investigate how multiple biomarkers associated with MDM2i sensitivity and insensitivity/resistance may be combined to enrich for drug response most effectively. Decision tree inputs included all multi-cancer biomarkers meeting Q-value thresholds of ≦0.5. Odds Ratios and p-values were computed by Fisher's exact test. TP53 mutation was selected as the first partitioned node. The subsequent nodes demonstrated the use of gene mutation biomarkers or custom gene signatures to achieve further enrichment.

Biomarker Frequencies in Cancer Subtypes

The frequency of TP53 mutation and sensitivity signature score ≧0.2 across cancer subtypes were compared. The frequency of TP53 mutation across cancer subtypes was obtained from the Oncomine Powertool Mutation Browser v2.0. Independently, the frequency of expression of the sensitivity signature (signature score ≧0.2) was determined using Compendia's custom Affymetrix U133 dataset. Independent patient cohorts were represented in the Oncomine Powertool Mutation Browser and the custom Affymetrix U133 dataset. Cancer subtypes with low frequency TP53 mutation and high frequency MDM2i sensitivity gene signature gene expression represent patient populations enriched for biomarkers predictive of MDM2i sensitivity.

Receiver Operating Characteristics of the Training Set and Leave-One-Out Cross-Validation (LOOCV) Sensitivity Signatures in TP53 Wild-Type Cell Lines

A LOOCV analysis was performed on all sensitive (n=62) and resistant (n=164) cell lines. For every cell line called sensitive or insensitive/resistant, a leave-one-out (LOO) signature was constructed by performing a differential expression analysis between the remaining sensitive and resistant lines. Each cell line was scored for each LOO signature, and the class of the left-out sample was predicted by comparing the left-out sample score to the mean signature scores of the remaining sensitive and resistant samples; if the left-out sample score was closer to the sensitive mean than the resistant mean, the left-out sample was classified as sensitive. The ability of the signature scores in the left-out samples to predict sensitivity was assessed using receiver operating characteristic (ROC) analysis.

An ROC plot was generated by plotting the true positive rate (y-axis) versus the false positive rate (x-axis) for Compound A training set and LOOCV sensitivity scores in TP53 wild-type cell lines. Wilcoxon p-values were calculated for each of the signature scores versus true sensitivity call status and found to be significant. The training set score and the LOOCV score had p-values of 8.3E-7 (i.e., 8.3×10⁻⁷) and 2.4E-4 (i.e., 2.4×10⁻⁴), and AUC values of 0.92 and 0.8, respectively. This analysis supports the predictive ability of the expression of genes in the gene signature indicative of sensitivity to the Compound A MDM2i.

The above-described results are summarized as follows: 139 genes of the 177 genes presented in FIGS. 1A-1E showed coherent and variable expression (i.e., R >0.2; Variance >0.2) in cancer types of interest, including multicancer. The thirty-eight genes presented in Table 3 showed consistent TP53-dependent expression both in vitro and in vivo, e.g., in preclinical animal tumor models; and the thirty-seven genes (i.e., the genes in Table 3 except for PEBP1) showed increased expression in cancer tissues relative to normal tissues. In Table 3, “all” refers to the following cancer types: acute lymphoblastic leukemia (ALL), acute myeloid leukemia (AML), diffuse large B cell lymphoma (DLBCL), glioblastoma (GBM), melanoma, multi-cancer and myeloma.

TABLE 3

| | Cancer Types with |
|---|---|
| Gene Signature | Positive Expression |
| Component Gene | Relative to Normal |
| BAX | all |
| C1QBP | all |
| FDXR | ALL, ALM, DLBCL, |
| | melanoma, myeloma, |
| | multi-cancer |
| GAMT | ALL, ALM, GBM, |
| | myeloma |
| RPS27L | DLBCL, GBM, melanoma, |
| | myeloma, multi-cancer |
| SLC25A11 | DLBCL, melanoma, |
| | myeloma |
| TP53 | all |
| TRIAP1 | all |
| ZMAT3 | all |
| AEN | all |
| C12orf5 | all |
| GRSF1 | all |
| EIF2D | ALL, ALM, DLBCL, |
| | melanoma, myeloma, |
| | multi-cancer |
| MPDU1 | AML, DLBCL, melanoma |
| STX8 | all |
| TSFM | DLBCL, myeloma |
| DISC1 | ALL, ALM, GBM, |
| | melanoma, myeloma, |
| | multi-cancer |
| PEBP1 | none |
| SPCS1 | all |
| PRPF8 | ALL, ALM, DLBCL, |
| | GBM, melanoma, |
| | multi-cancer |
| RCBTB1 | all |
| SPAG7 | AML, myeloma |
| TIMM22 | ALL, GBM, melanoma, |
| | myeloma |
| TNFRSF10B | all |
| ACADSB | ALL, ALM, melanoma, |
| | myeloma, multi-cancer |
| DDB2 | ALL, ALM, DLBCL, |
| | melanoma, myeloma, |
| | multi-cancer |
| FAS | AML, DLBCL, GBM |
| GDF15 | GBM, melanoma |
| GREB1 | ALL, GBM, melanoma, |
| | myeloma, multi-cancer |
| PDE12 | ALL, AML, melanoma, |
| | myeloma, multi-cancer |
| POLH | all |
| C19orf60 | myeloma |
| HHAT | ALL, AML, melanoma, |
| | multi-cancer |
| ISCU | myeloma |
| MDM2 | all |
| MED31 | ALL, AML, myeloma |
| METRN | GBM, melanoma |
| PHLDA3 | melanoma |

### Example 3

### Gene Expression Profile Refinement Associated with MDM2i Sensitivity

The gene signatures indicative of sensitivity to MDM2i were further refined in an effort to determine a gene set that was highly correlated with MDM2i sensitivity in a variety of cancer types/subtypes. Computer software, algorithms and bioinformatics methods were utilized.

As will be appreciated by the skilled practitioner in the art, Random Forests is a machine learning algorithm, as described by L. Breiman (2001, Machine Learning, 45, 5-32), for classification and regression analysis. The algorithm works by constructing many decision trees consisting of repeatedly and randomly selected samples and variables from original data. After a Random Forests model is created, the model can be simplified by excluding variables that are not important for classification. The variable selection method was developed by R. Diaz-Uriarte et. al. (2006, BMC Bioinformatics, 7(3):1471-1421) and can select important variables from a Random Forests model using both backwards variable elimination and selection based on the variable importance score. These techniques were applied to the sensitivity data of cell lines in OncoPanel™, e.g., Example 2, to select gene set that would contribute to the effective classification of their sensitivities.

As an initial gene set, 350 genes provided by Compendia were used. These genes were selected genes as sensitivity- and resistance-signature genes out of 175 total genes identified from the analysis of the MDM2i Compound A as described in Example 1. The sensitivity score was binarized as “sensitive” and “resistant”; 70 cell lines with an IC₅₀ value of less than or equal to 2 μM (≦2 μM) were defined as sensitive, and 163 cell lines with an IC₅₀ value of greater than or equal to 20 μM (≧20 μM) were defined as resistant. The 7 cell lines with marginal sensitivity, i.e., having an IC₅₀ value of between 2 μM and 20 μM, were removed from the analysis. Messenger RNA (mRNA) expression values, which were also provided by Ricerca Biosciences, were used as explanatory variables.

A Random Forests model was created by a commercially available “randomForest” package (ver. 4.6) of R statistics software (ver. 2.13). The parameter for the number of trees was set to 5000. The variable selection algorithm was implemented in “varSelRF” package (ver. 0.7) of R. Once the object of randomForest was created using the initial data, the varSelRF method was consecutively applied to it. As a result, eight genes (BAX, CDKN1A, DDB2, EDA2R, FDXR, MDM2, RPS27L, and SPATA18) were chosen as significant genes for MDM2i sensitivity classification. The BAX, CDKN1A, DDB2, FDXR, MDM2 and RPS27L genes are also included in the gene set of 38 genes shown in. Table 3. In order to evaluate whether the eight genes were sufficient to classify the cell lines as sensitive to MDM2i, the out-of-bag (OOB) error estimate values were compared between the models created using the original 350 genes and the selected 8 genes. The OOB error rate of the 350-gene model was 10%, while that of 8-gene model was 9.4%, indicating that decreasing the number of genes from 350 to 8 did not affect the performance of the prediction model to predict MDM2i sensitivity.

### Example 4

### Gene Signatures Predict Sensitivity to MDM2i Treatment of Tumored Animals in In Vivo Human Tumor Graft Model Study

This Example describes in vivo experiments using tumored animal models demonstrating that gene signatures based on the invention and the sensitivity signature scores related thereto were effective in predicting the sensitivity of various tumor types to a specific MDM2i in animals having such tumors and treated with the MDM2i.

Materials and Methods

Patient-derived xenograft models (Champions TumorGraft™; Champions Oncology, Inc., Hackensack, N.J.) were used. Champions TumorGrafts™ provide a highly focused, accelerated translational platform, which is based upon the implantation of primary human tumors in immune-deficient mice followed by propagation of the resulting low-passage Champions TumorGrafts™ in a manner that preserves the biological characteristics of the original human tumor. According to information provided by the company, histologic and molecular studies have shown that Champions TumorGraft™ models maintain the fundamental genotypic and phenotypic features of the original tumor including cancer stem cells and stroma; represent the genetic heterogeneity of cancer; predict the effectiveness of oncology drugs in patients; allow for the identification of highly responsive patient populations; do not change genetically over multiple passages; correlate genetically with the original patient tumor; and exhibit consistent growth and response to standard agents. The TumorGrafts™ models allow a comparison of gene expression analysis of patients' cancer samples and Champions TumorGraft™ samples by microarray expression analysis. Large numbers (e.g., 30,000) of genes are analyzed. A Pearson correlation shows a high percentage (e.g., 94%) correlation between cancer gene expression in the tumor graft and in the original tumor.

Animals

Female immunocompromised nu/nu mice (Harlan) between 6-9 weeks of age were housed on irradiated papertwist-enriched ⅛″ corncob bedding (Sheperd) in individual HEPA ventilated cages (Innocage® IVC, Innovive USA) and were kept on a 12-hour light-dark cycle at 68-74° F. (20-23° C.) and 30-70% humidity. Animals were fed water ad libitum (reverse osmosis, 2 ppm C12) and an irradiated Test rodent diet (Teklad 2919) consisting of 19% protein, 9% fat, and 4% fiber.

Sensitivity Score Calculation

Sensitivity and resistance signatures were scored in Champions TumorGraft™ models. To characterize relative signature expression within the models, a custom dataset was created that combined all Champions TumorGraft™ models measured on Affymetrix U219 microarray format platforms (>145 models). Gene expression data were processed using the GC Robust Multi-array Average (GCRMA) background adjustment algorithm. Alternative chip definition files (aItCDF) were used to summarize probes into probe sets associated with Entrez Gene identifiers. As is appreciated by the skilled practitioner, the HGU219_Hs_ENTREZG alternative CDF (version 15.1.0), which is available and downloadable via the following internet address (i.e., http://brainarray.mbni.med.umich.edu/Brainarray/Database/CustomCDF/genomic_curated_CDF.asp), was utilized. To normalize the contribution of each gene in the signature, signature genes were subject to Z-score normalization (the mean expression value of each gene is subtracted from the value within each sample, and the difference is divided by the standard deviation). Signature scores across the models were generated by determining the unweighted average of the Z-score normalized expression values of each signature gene.

Description of Tumor Models

Animal tumor models for several human tumor tissue types were produced as described below and presented in Table 5 shown below. NSCLC refers to non small cell lung cancer. The signature score is a representative MDM2i gene sensitivity signature score or value obtained from the analysis of 177 genes (the genes presented in FIGS. 1A-1E), 175 genes (the genes presented in FIGS. 1A-1E, except for EDA2R and SPATA18), 40 genes (the genes presented in Table 1), 4 genes (RPS27L, FDXR, CDKN1A and AEN) and 3 genes ((RPS27L, FDXR and CDKN1A) correlating with the level of sensitivity of each tumor type to the MDM2i used in the study, namely, Compound B (3′R,4′S,5′R)—N-[(3R,6S)-6-carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrolidine-3′,3″-indole]-5′-carboxamide, and Compound B p-toluenensulfonate, in this Example.

Tumor Implantation

Animals were implanted bilaterally in the flank region with tumor fragments harvested from tumored host animals, each implanted from a specific passage lot. Pre-study tumor volumes were recorded for each experiment beginning approximately one week prior to its estimated start date. When tumors reached approximately 125-250 mm³, animals were matched by tumor volume into treatment and control groups, and dosing was initiated (Day 0). Animals in all studies were tagged and followed individually throughout the experiment.

Dosing Regimen

Initial dosing for standard agents began on Day 0; animals in all groups were dosed by weight (0.01 ml per gram; 10 ml/kg). Dose concentration(s), route(s) of administration and schedule(s) for each group are listed in the Experimental Design section, wherein “p.o./qd×10” indicates orally (by mouth) daily for 10 days.

Experimental Design

The experimental design of the human tumor graft model study is presented below in Table 4. “n” indicates the number of animals per group; “ROA” indicates route of administration of the test agent, i.e., the MDM2i drug.

TABLE 4
Champions TumorGraft ™ Models of Human
Melanoma, NSCLC, Colorectal, and Pancreatic Cancers
Treated with MDM2i, Compound B p-toluenesulfonate

| | | | Dose | |
|---|---|---|---|---|
| Group | -n- | Agent | (mg/kg/dose) | ROA/Schedule |
| 1 | 10 | Vehicle Control | — | p.o./qd × 10 |
| 2 | 10 | Compound B | 100 | p.o./qd × 10 |
| | | p-toluene-sulfonate | | |

### Assessment of Test Agent Efficacy

Tumor Growth Inhibition (TGI):

Beginning on Day 0, tumor dimensions were measured twice weekly by digital caliper and data including individual and mean estimated tumor volumes (Mean TV±SEM) were recorded for each group. Tumor volume was calculated using the formula (1): TV=width²×length×0.52. At study completion, percent tumor growth inhibition (% TGI) values were calculated and reported for each treatment group (T) versus control (C) using initial (i) and final (f) tumor measurements and the formula (2): % TGI=1−T_{f}−Tᵢ/C_{f}−C. The tumor growth inhibition (TGI) values on the scheduled dates closest to the last administration are summarized Table 5 below. In this example, the study duration was defined by the tumor size of the vehicle control. In some cases, for example, that of CTG-0213 in Table 5, required a longer time period for the tumor to reach a threshold size. As a result, the test animals treated with the MDM2i Compound B were also left for a longer time period, e.g., 40 days in the case of CTG-0213, without MDM2i treatment. The length of time without MDM2i treatment caused tumor regrowth in some of the CTG-0213 animals. Thus, the evaluation of TGI was set to be near the end of the treatment period, such as around Day 7-11 (around Day 9).

A comparison of the signature score values with % TGI in Table 5 shows that tumor types having a high signature score correlated with a high percentage of tumor growth inhibition at the time of TGI evaluation.

TABLE 5

| | Tumor | | | | | | Study | | |
|---|---|---|---|---|---|---|---|---|---|
| Model | Tissue | Signature Score Values | | | | | Duration | TGI | |
| Name | Type | 177 | 175 | 40 | 4 | 3 | (days) | evaluation | % TGI |
| CTG-0201 | Melanoma | 0.0 | 0.0 | −0.5 | −0.6 | −1.0 | 17 | Day 10 | −10 |
| CTG-0204 | Melanoma | 0.8 | 0.7 | 0.9 | 0.7 | 0.6 | 17 | Day 10 | 51 |
| CTG-0213 | Melanoma | 0.2 | 0.2 | 0.1 | 0.2 | 0.3 | 49 | Day 7 | 87 |
| CTG-0500 | Melanoma | 0.3 | 0.3 | 0.0 | 0.6 | 0.1 | 13 | Day 7 | 104 |
| CTG-0501 | Melanoma | 0.6 | 0.5 | 0.9 | 0.7 | 0.3 | 18 | Day 11 | 101 |
| CTG-0069 | Colorectal | −0.3 | −0.2 | −0.5 | −0.8 | −0.8 | 20 | Day 11 | 19 |
| CTG-0093 | Colorectal | 0.4 | 0.4 | 0.6 | 1.0 | 1.2 | 23 | Day 9 | 53 |
| CTG-0159 | NSCLC | −0.2 | −0.2 | −0.2 | −1.5 | −1.6 | 18 | Day 11 | −13 |
| CTG-0502 | NSCLC | 0.6 | 0.6 | 0.9 | 0.9 | 0.8 | 18 | Day 10 | 122 |
| CTG-0282 | Pancreatic | 0.2 | 0.2 | 0.6 | 1.2 | 1.0 | 19 | Day 11 | −30 |
| CTG-0292 | Pancreatic | −0.2 | −0.2 | −0.4 | −0.7 | −0.7 | 21 | Day 11 | 54 |
| CTG-0203 | Melanoma | 0.7 | 0.7 | 0.6 | 0.6 | 0.7 | 25 | Day 11 | 98 |

### Example 5

### Prediction of Sensitivity to MDM2i Treatment by Using 20 Samples Whose MDM2i Sensitivity is Unknown as a Training Set

CCLE Datasets

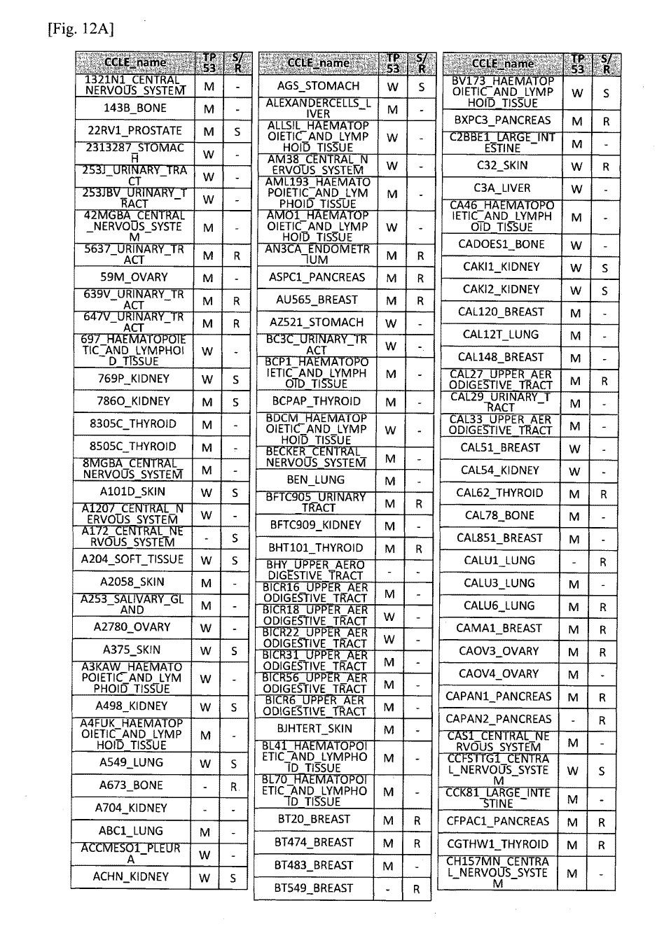

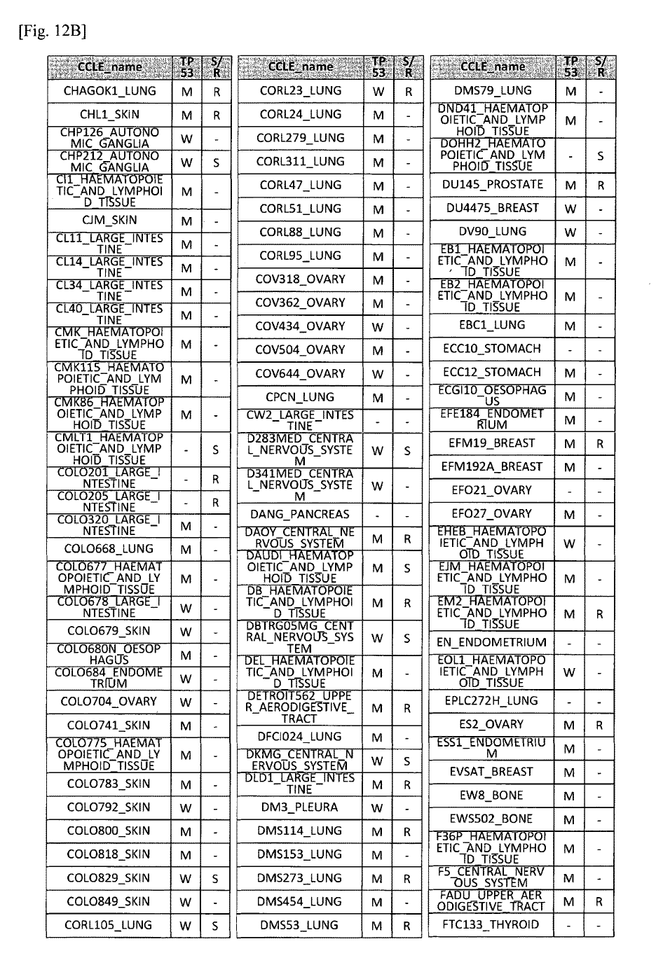

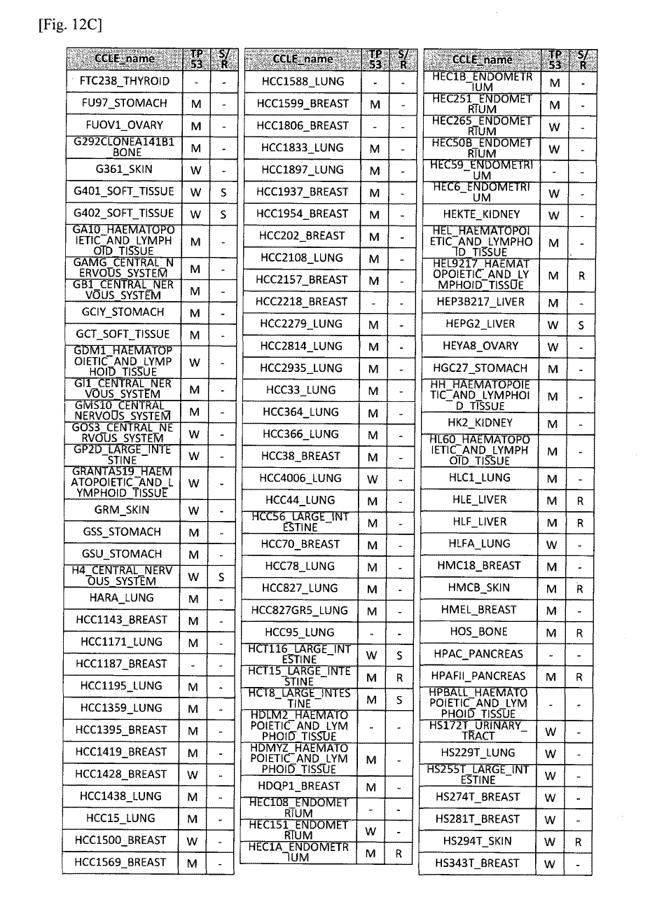

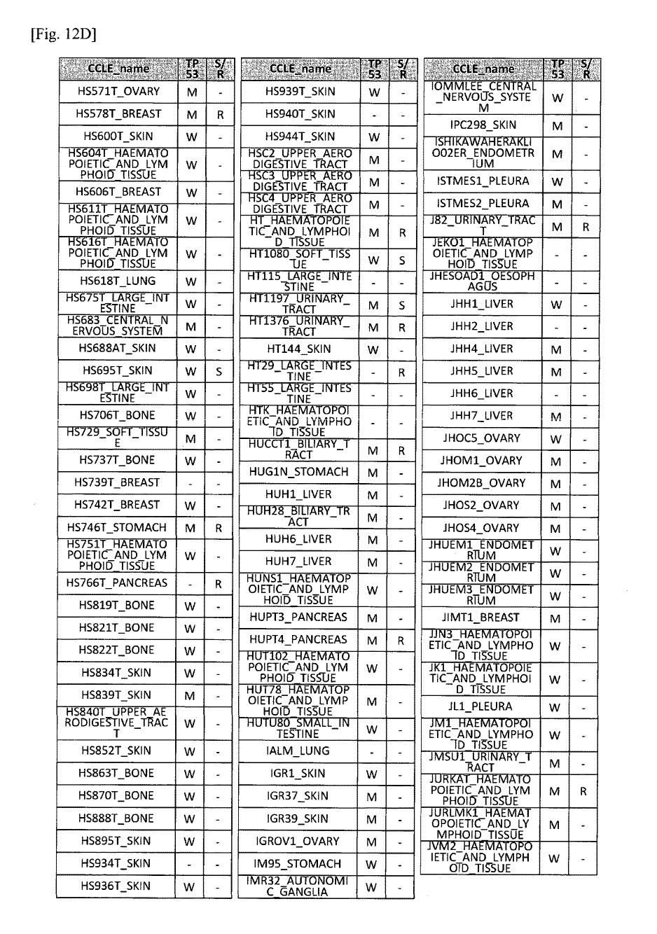

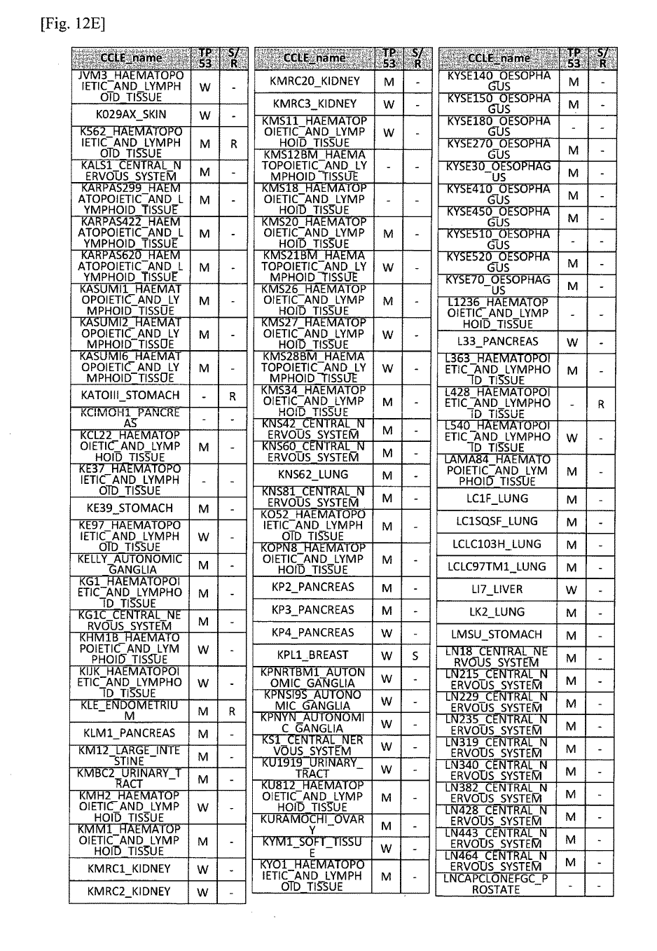

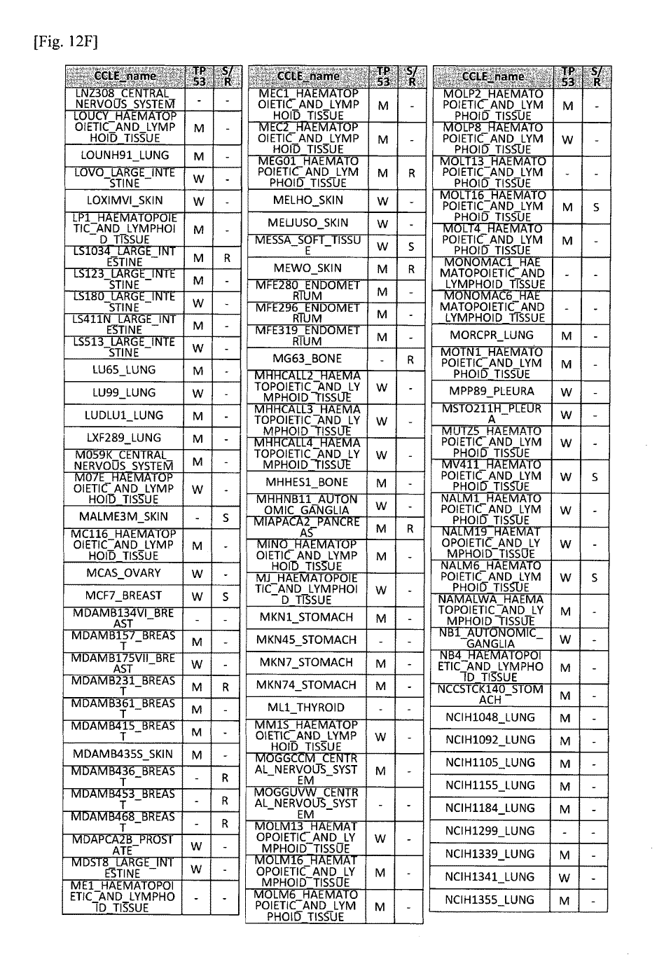

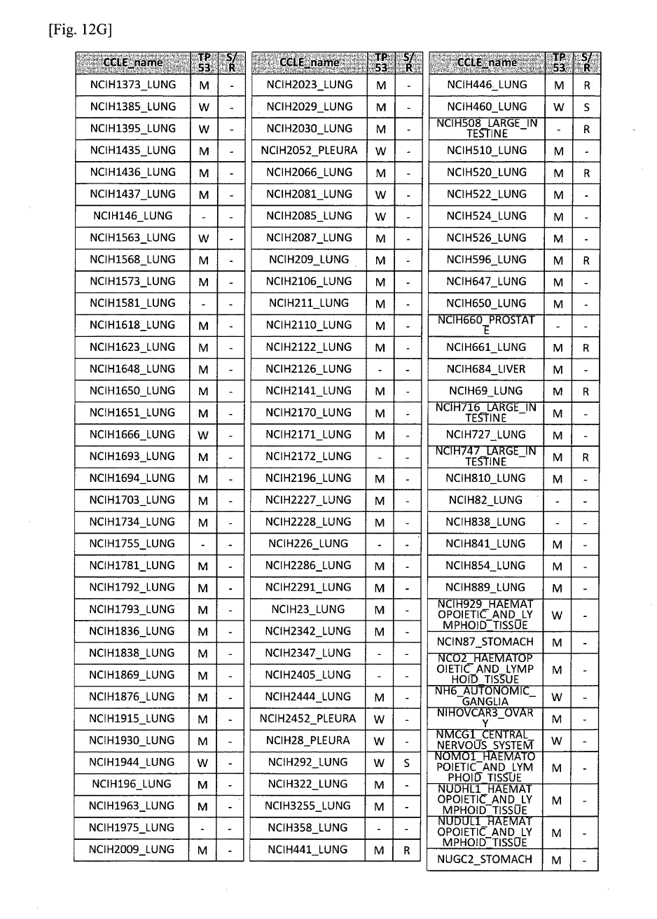

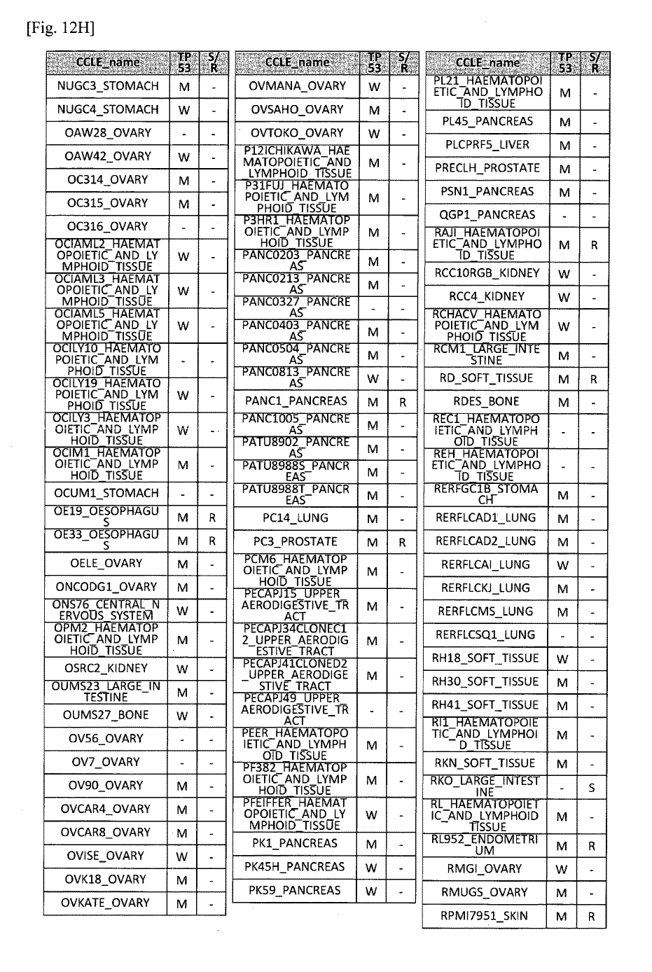

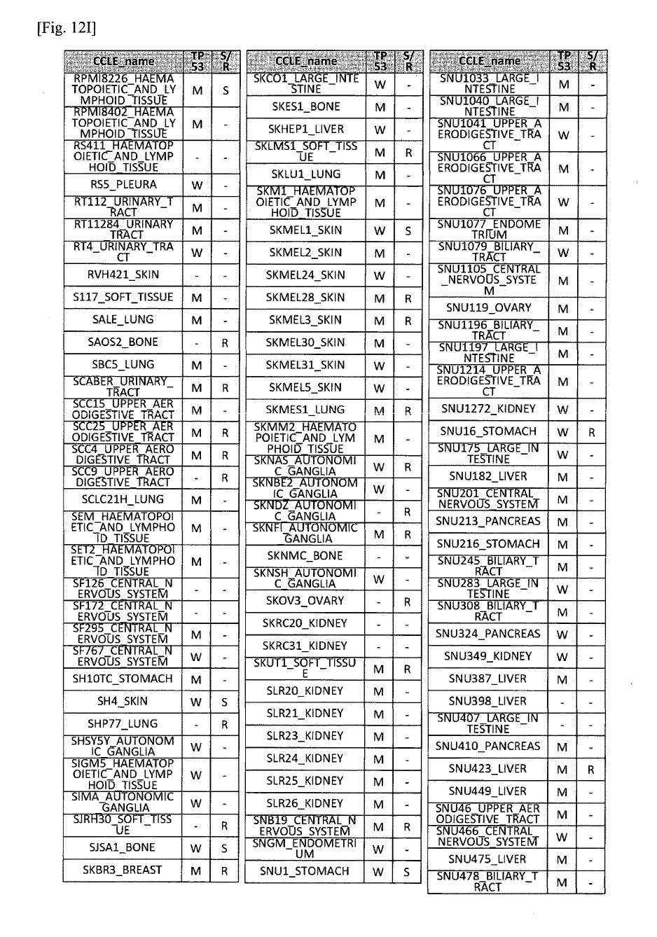

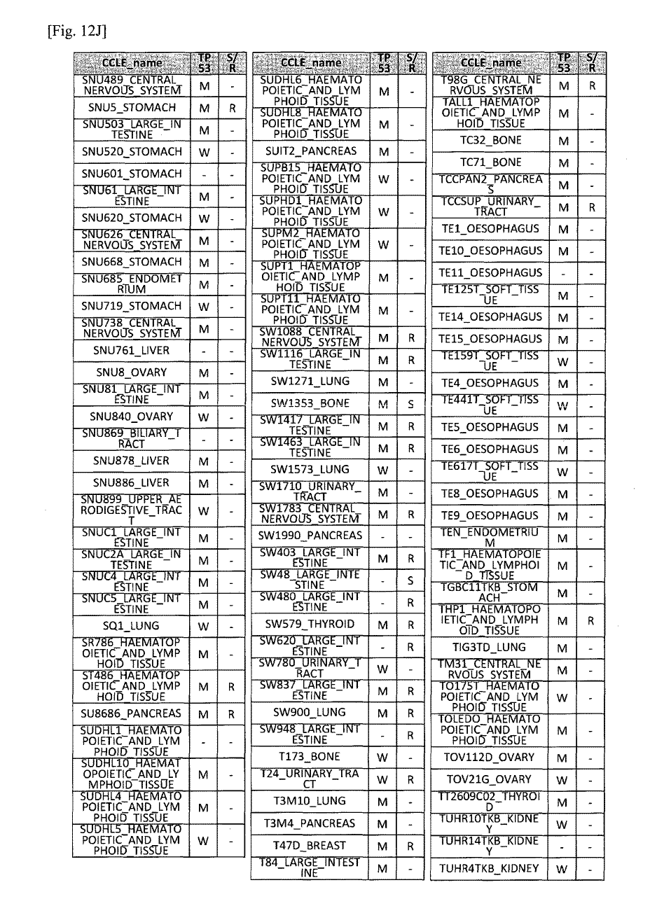

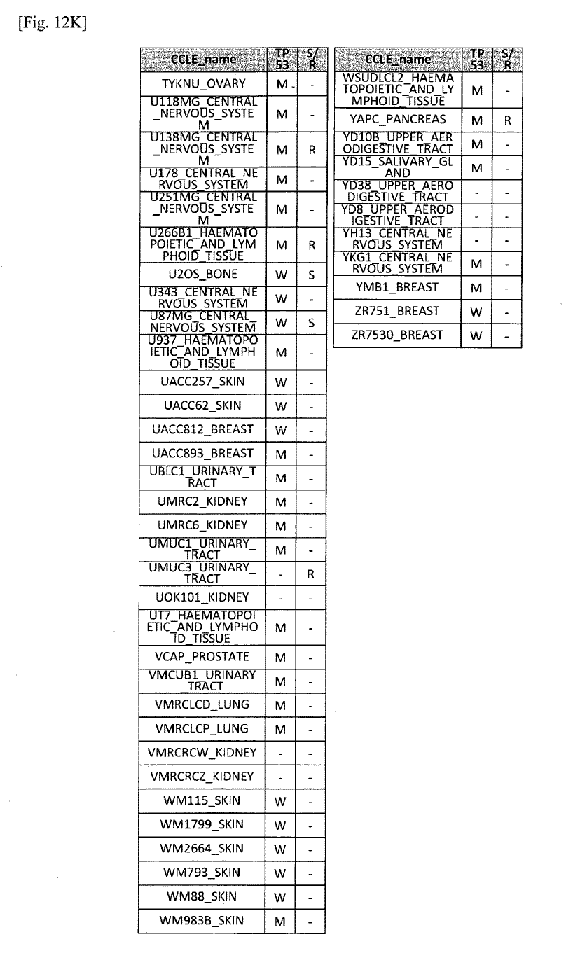

In order to predict the sensitivity to MDM2 inhibitor treatment of a cancer cell line from samples having no pharmacological data as a training set, we used mRNA expression data of cell lines by using Cancer Cell Line Encyclopedia (CCLE), which is provided by the Broad Institute and the Novartis Institutes for Biomedical Research and its Genomics Institute of the Novartis Research Foundation. We found 185 cell lines with MDM2i sensitivity data, 242 p53 wild type cell lines without MDM2i sensitivity data, and 512 p53 mutant cell lines without MDM2i sensitivity data in the CCLE datasets. The cell lines of CCLE database used in the prediction are shown in FIG. 12.

Patient-Derived Xenograft Models (Champions TumorGraft™; Champions Oncology, Inc., Hackensack, N.J.)

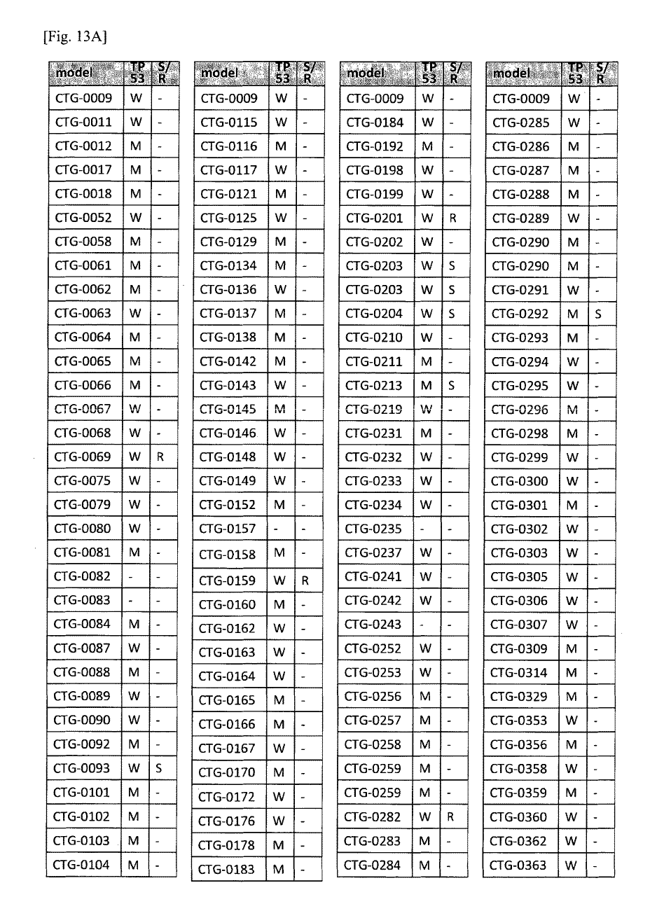

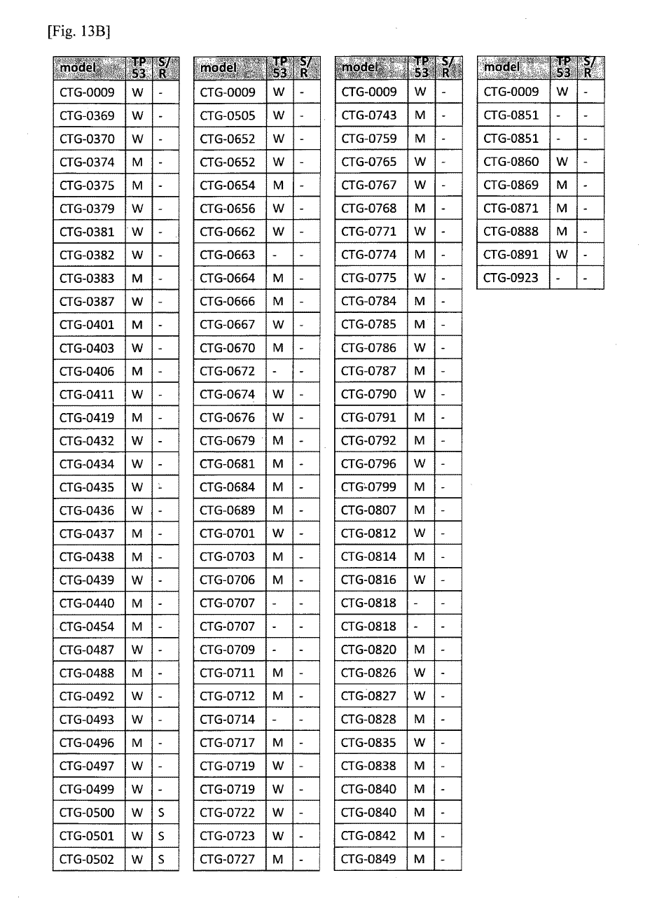

PDx models, which were listed in FIG. 13, were purchased from Champions Oncology, Inc. PDx models with the patient's tumor closely resemble its features have been developed in order to examine the effect of a drug of interest with a high likelihood of exhibiting the same effect against the tumor in the human body. In particular, PDx models are generated by grafting the patient's tumor containing stroma and cancer stem cells into mice, and therefore closely resemble the patient's tumor, while traditional tumor models are generated by grafting a tumor cell line established from the patient's tumor into mice, strongly reflecting the grafted tumor cell line's feature. The PDx models used in the prediction are shown in FIG. 13. The PDx models included 12 models with MDM2i sensitivity data, 105 p53 wild type models without MDM2i sensitivity data, and 103 p53 mutant models without MDM2i sensitivity data.

TP53 Mutation Information

TP53 mutation information was downloaded from the web site of Broad-Novartis Cancer Cell Line Encyclopedia (CCLE) (http://www.broadinstitute.org/ccle/home), Sanger COSMIC Cell Line Project (CLP) (http://cancer.sanger.ac.uk/cancergenome/projects/cell_lines/), and International Agency for Research on Cancer (IARC) (http://www.iarc.fr/). Hybrid capture sequencing data of CCLE was the version of 22 May 2012, CLP was version 69, and IARC TP53 Database was version R17. Because some inconsistency existed among these databases, we labeled ‘wild type’ (wt) and ‘mutant’ (mut) to each cell line only when all these databases have consistent mutation information. For example, when a cell line was ‘mutant’ in both CLP and IARC but ‘wild type’ in CCLE, the mutation status of the cell line was determined as unknown. In case of a cell line had no information in one or two of the databases, the mutation status of the cell was determined based on the other databases. For example, when a cell line was ‘wild type’ in both CCLE and IARC but no information in CLP, the cell line was labeled as ‘wild type’.

Sensitivity to MDM2 Inhibitor

IC₅₀, the concentration where 50% inhibition of cell growth was observed, was determined by Ricerca Biosciences (Eurofins). Sensitivity to MDM2 inhibitor was determined by referring to the IC₅₀, as sensitive if IC₅₀<=1 μM and as resistant if IC₅₀>1 μM in this Example.

Prediction

The sensitivity to MDM2i treatment was predicted in each of 185 cell lines with MDM2i sensitivity data. The prediction was performed by using the expression profile of the 4 or 40 genes in 20 cell lines, as a training set, randomly picked up from the cell lines without MDM2i sensitivity data. The prediction was repeated 100 times using a different training set in each time. Then, predicted results were compared with the experimentally determined sensitivity data to know whether the prediction was accurate or not. In order to examine the effect of TP53 mutation in the prediction accuracy, the numbers of TP53 wild type cells and TP53 mutant cells were selected so that each training set has the indicated number of TP53 wild type cell lines and TP53 mutant cell lines in each figures.

Prediction Accuracy

In addition to the FPR and FNR, prediction accuracy was calculated. When a sensitive cell line was predicted as sensitive or a resistant cell line was predicted as resistant, the prediction was evaluated as correct. After the 100 simulations, the percentage of correct predictions was calculated for each cell line.

Melanoma

To focus on melanoma cell lines, 10 TP53 mutated melanoma cell lines and 10 TP53 wild type melanoma cell lines were randomly picked out from the CCLE datasets to create a 20 melanoma cell line set. The CCLE datasets include 185 cell lines with MDM2i sensitivity data and 29 p53 wild type cell lines without sensitivity data and 14 p53 mutant cell lines without sensitivity data.

Lymphoma

To examine the predictability on lymphoma cell lines, 10 TP53 mutated melanoma cell lines and 10 TP53 wild type melanoma cell lines were randomly picked out from the CCLE datasets to create a 20 lymphoma cell line set. The CCLE datasets include 185 cell lines with MDM2i sensitivity data and 44 p53 wild type cell lines without sensitivity data and 60 p53 mutant cell lines without sensitivity data.

Score Extrapolation Models

After obtaining the expression profile of the genes in the cell lines, the amount of expression of each gene was normalized so that the average is 0 and the standard deviation (SD) is 1 to obtain a normalized score of each gene. The score of a sample was calculated by averaging the normalized scores of all of the genes analyzed. In order to predict whether the sample of interest is sensitive or resistant, optimized thresholds, which maximize a true positive rate and minimize a false positive rate, are determined by plotting Receiver Operating Characteristic (ROC) plots and conducting leave-one-out cross-validation (LOOCV) analysis. −0.02 (from LOOCV) and 0.2 (from original analysis) are obtained as a threshold and are referred to as “fixed(−0.02)” and “fixed(0.2)”, respectively. Threshold was also determined by Otsu's method (see M. Sezgin and B. Sankur (2004), Journal of Electronic Imaging 13 (1): 146-165, and N. Otsu (1979), IEEE Trans. Sys., Man., Cyber. 9 (1): 62-66), which is referred to as a “score distribution” model in Figures. Threshold was also determined as the value of the first quartile of the reference scores of TP53 wild type samples among the samples, which is referred to as a “score in wt” model in Figures.

### Gaussian Mixture Models (Also Referred to as “High-Percentage Models” Hereinafter)

In order to create Gaussian mixture models, a commercially available “mclust” package (ver. 4.3), which was developed by C. Fraley et. al. (Technical Report no. 597, Department of Statistics, University of Washington, June 2012) was used on R statistics software (ver. 3.0.2). Gaussian mixture models can be created as follows. In a cell line panel that consists of sensitive and resistant cell lines, the distribution of mRNA expression of a signature gene can be described as a mixture of the distribution derived from sensitive cell lines and resistant cell lines. If the distributions are supposed to be normal distribution, the mixed distribution is described as the Gaussian mixture model:

[Math. 8]

p(x|λ)=Σᵢ₌₁²ωᵢg(x|μᵢ,σ). (1)

λ is a set of parameters: λ={ωᵢ, μᵢ, σ}, i=1, 2 and ωᵢ, i=1, 2 are the mixture weights, and g(x|μᵢ, σ), i=1, 2 are the component Gaussian densities. Each component density is a Gaussian function of the form,

$$\begin{matrix}[ Math . 9 ] & \\ g ( x \vert {μ}_{i} , σ ) = \frac{1}{\sqrt{2 {πσ}^{2}}} exp ( - \frac{{( x - {μ}_{i} )}^{2}}{2 {σ}^{2}} ) , i = 1 , 2 , & ( 2 )\end{matrix}$$

with mean μᵢ, i=1, 2. For convenience, μᵢ satisfy the constraint that μ₁<μ₂. The standard deviation σ is supposed to be common between sensitive and resistant cell lines σ=σ₁=σ₂. Each parameter can be estimated by maximum likelihood estimation, which is to find model parameters maximizing the likelihood of the model given the training data. For a sequence of T training vector X={x₁, . . . , x_{T}}, the likelihood can be written as,

[Math. 10]

p(X|λ)=Π_{z=1}^{T}p(xₜ|λ). (3)

Maximum likelihood parameters can be obtained by Expectation-Maximization (EM) algorithm. The basic idea of the EM algorithm is to estimate a new parameter \hat{λ} from the previous parameter λ such that p(X|\hat{λ})≧p(X|λ). The X is repeatedly renewed until the likelihood converges on a maximum value. For M genes, when the expression of each gene is written as xₘ, m=1, . . . , M, the class of gene m can be written as,

$$\begin{matrix}[ Math . 11 ] & \\ {C}_{m} = {argmax}_{i} \{ {ω}_{i} g ( {x}_{m} \vert {μ}_{i} , σ ) \} , i = 1 , 2 , m = 1 , … , M , & ( 4 )\end{matrix}$$

where Cₘ is 1 or 2, and we call the gene is ‘lower’ when Cₘ=1 and ‘upper’ when Cₘ=2. When a variable,

$$\begin{matrix}[ Math . 12 ] & \\ {u}_{m}^{{c}_{m}} = \{ \begin{matrix}0 , & {C}_{m} = 1 \\ 1 , & {C}_{m} = 2\end{matrix} , & ( 5 )\end{matrix}$$

is introduced to describe the class to which the gene m belongs, the ‘upper ratio’ is given by

$$\begin{matrix}[ Math . 13 ] & \\ ( upper ratio ) = \frac{{\sum }_{m = 1}^{M} {u}_{m}^{{C}_{m}}}{M} . & ( 6 )\end{matrix}$$

A threshold of a sensitivity score to MDM2 inhibitor was determined by referring to the upper ratio (6). In order to determine the threshold of the upper ratio for discrimination, gene expression level of cell lines with TP53 mutation was used. This strategy is possible because most of the cell lines with TP53 mutation are resistant to MDM2 inhibitors. The threshold was determined as the level of the third quartile (“High %[quartile]” or simply “quantile”) or the maximum (“High %[max]” or simply “max”) of the upper ratio when TP53 mutant cell lines were ordered by their upper ratio. A cell line is predicted as sensitive when the upper ratio is higher the threshold, and is predicted as resistant when the upper ratio is lower than the threshold.

$$\begin{matrix}[ Math . 14 ] & \\ ( Likelihood \cdot ratio ) = {\prod }_{m = 1}^{M} \frac{{ω}_{2} g ( {x}_{m} \vert {μ}_{2} , σ )}{{ω}_{1} g ( {x}_{m} \vert {μ}_{1} , σ )} & ( 7 )\end{matrix}$$

Sensitivity to MDM2 inhibitor was also determined by referring to the likelihood ratio (7), as sensitive if the ratio >=1 and as resistant if the ratio <1, which is referred to as “distribution-only” model in Figures.

Simulation

For each set of the 20 cell lines, 2 prediction models were created: one was the model whose sensitivity threshold was determined from the max value of the TP53 mutated cell lines (‘max’ model), and the other was the model whose sensitivity threshold was determined from the 75 percentile (third quartile) of the TP53 mutated cell lines (‘quartile’ model). Each prediction model was applied to 185 cell lines and calculated false positive rate (FPR) and false negative rate (FNR) as follows:

[Math. 15]

FPR=FP/(FP+TN) (8)

FNR=FN/(TP+FN) (9)

wherein TP is the number of true positive cases, TN is the number of true negative cases, FP is the number of false positive cases, and FN is the number of false negative cases. This simulation was also repeated 100 times and the results were plotted on a graph where X-axis and Y-axis represent FPR and FNR, respectively.

Results

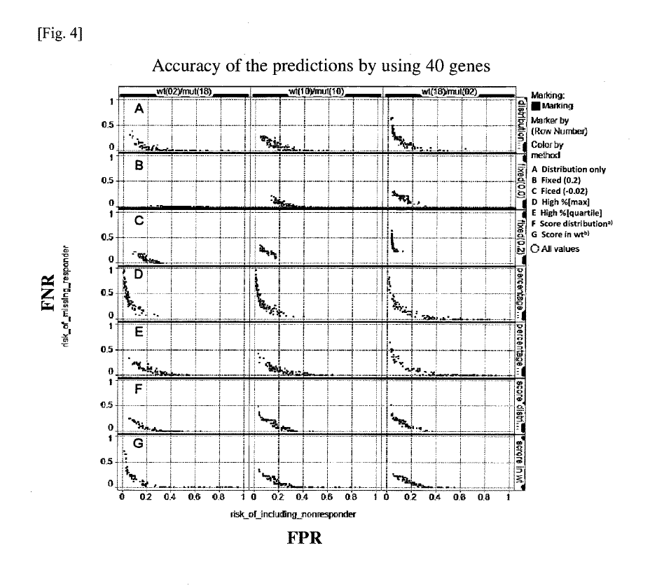

As shown in FIG. 4, sensitivity to MDM2i treatment was well predicted by any of the prediction models using training sets where sensitivities of any cell lines in the training sets are unknown. The “fixed (0.2)” model best predicted the sensitivity of cancer cells to MDM2i treatment regardless of what training set was used and regardless of TP53 mutation. The prediction using only 4 genes also well predicted the sensitivity of cancer cells to MDM2i treatment (data not shown).

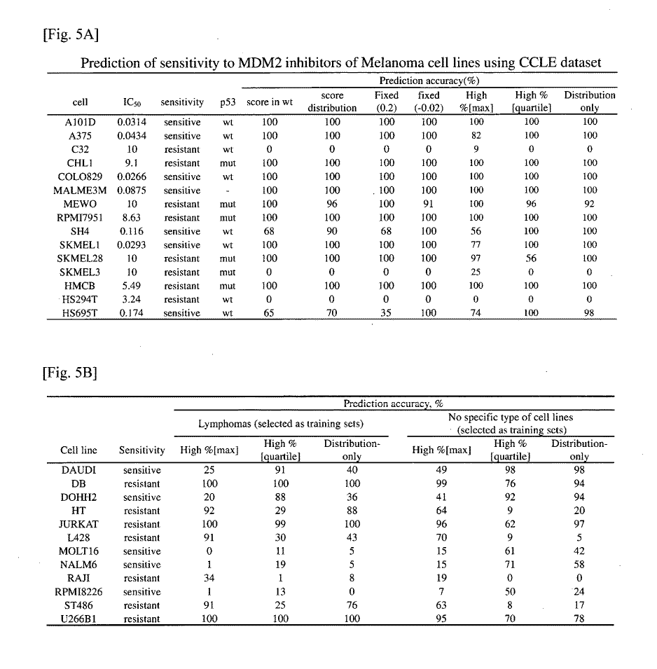

The results of the prediction of sensitivities of melanoma cell lines using only melanoma cell lines as training sets were shown in FIG. 5A. In melanoma cell lines, all prediction models highly precisely predicted the sensitivity of the melanoma cell lines. These results indicate that prediction accuracy can be improved by excluding other types of cancers and that sensitivity to MDM2i treatment can be precisely predicted in melanomas.

The results of the prediction of sensitivities of lymphoma cell lines where lymphoma cell lines or no specific type of cell lines were used as training sets were shown in FIG. 5B. As shown in FIG. 5B, all prediction models precisely predicted the sensitivity of the lymphoma cell lines. These results also indicate that prediction accuracy can be improved by excluding other types of cancers from training sets.

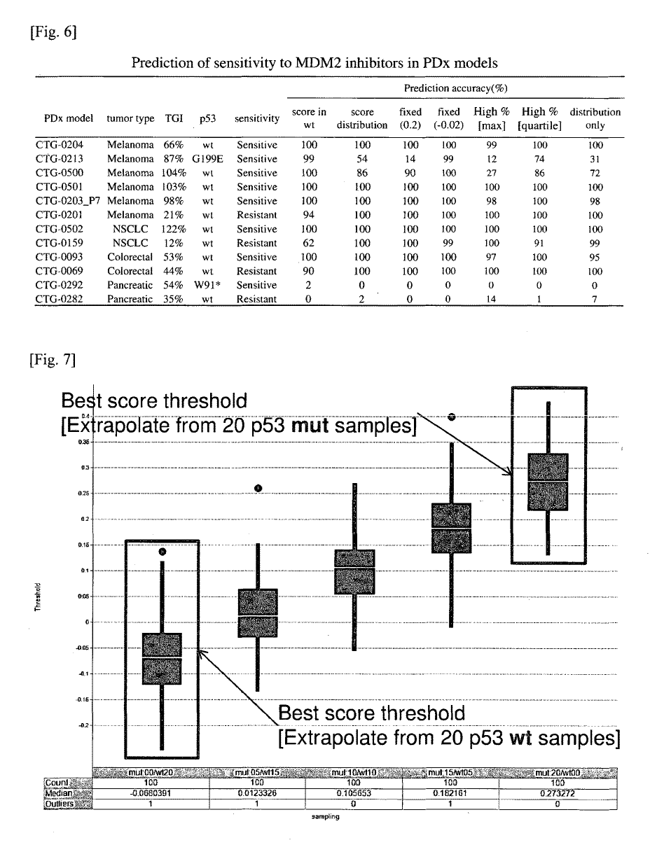

To confirm the results, sensitivity to MDM2i treatment was predicted in PDx models. As shown in FIG. 6, all of the prediction models precisely predicted the sensitivity of the tumor in PDx models, which have an in vivo cancer environment.

### Example 6

### Effect of p53 Mutation in Cancers on Predictability of their Sensitivities to MDM2i Treatment

20 cancer cell lines were randomly picked out from CCLE datasets and used as a training set. In order to evaluate the effect of p53 mutation in cancers on the predictability, 0, 5, 10, 15 or 20 cancer cell lines with p53 mutation were included in each training set. The optimized thresholds, which minimize false positive rate and maximize true positive rate, were determined by LOOCV analysis in each training set. Each optimized threshold was repeatedly obtained from 100 different training sets. These optimized thresholds were plotted on FIG. 7.

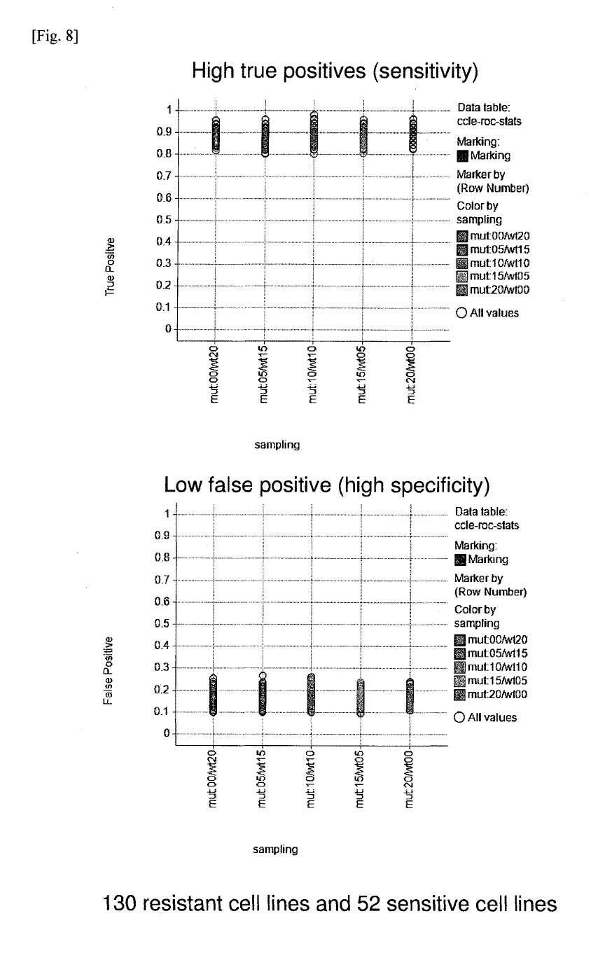

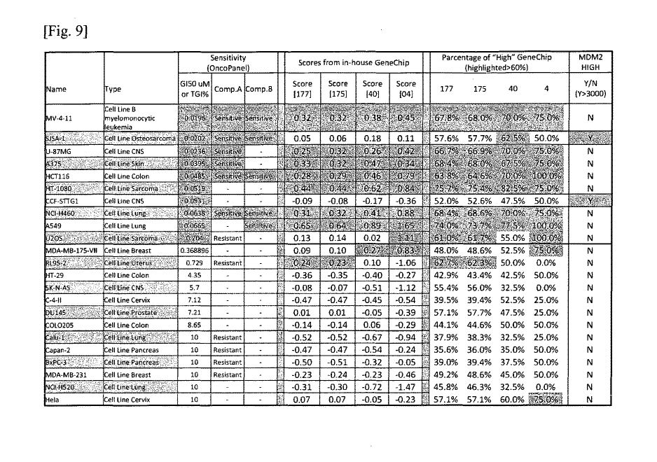

As shown in FIG. 7, the distribution of the threshold tends to increase when a training set contains increased number of p53 mutants. The sensitivity was then predicted with a score extrapolation model as described in Example 5 by using each of the optimized thresholds. Surprisingly, the sensitivity was precisely predicted regardless of p53 mutation rate in a training set. These results indicate that p53 mutation rate in a training set may affect the threshold value, but do not affect the accuracy of the prediction (see FIG. 8).

This Example also shows that sensitivity can be predicted by using cell lines whose sensitivity is totally unknown.

### Example 7

### Effect of MDM2 Gene Amplification in Cancers on Predictability of their Sensitivities to MDM2i Treatment

The prediction was performed by score extrapolation models and Gaussian mixture models as described in Example 5. Using top 4, top 40, top 175 or top 177 genes in FIGS. 1A to 1E as prediction markers results in a good prediction of the sensitivity (see FIG. 9). However some of the sensitive cell lines such as SJSA-1 and CCF-STTG1 were predicted as resistant in FIG. 9.

In order to evaluate the impact of MDM2 gene amplification (or subsequent overexpression of the gene) on the sensitivity prediction, we compared prediction accuracies calculated from the results obtained by using gene expression values of 4 genes (RPS27L, FDXR, CDKN1A and AEN) in a hundred training sets with or without using MDM2 expression level as explanatory variables by linear discriminant analysis. Leave one out cross validation of the 23 cell lines, which were listed in FIG. 9, was done and each cell was predicted as sensitive (probability of sensitivity >=0.5) or resistant (probability of sensitivity <0.5).

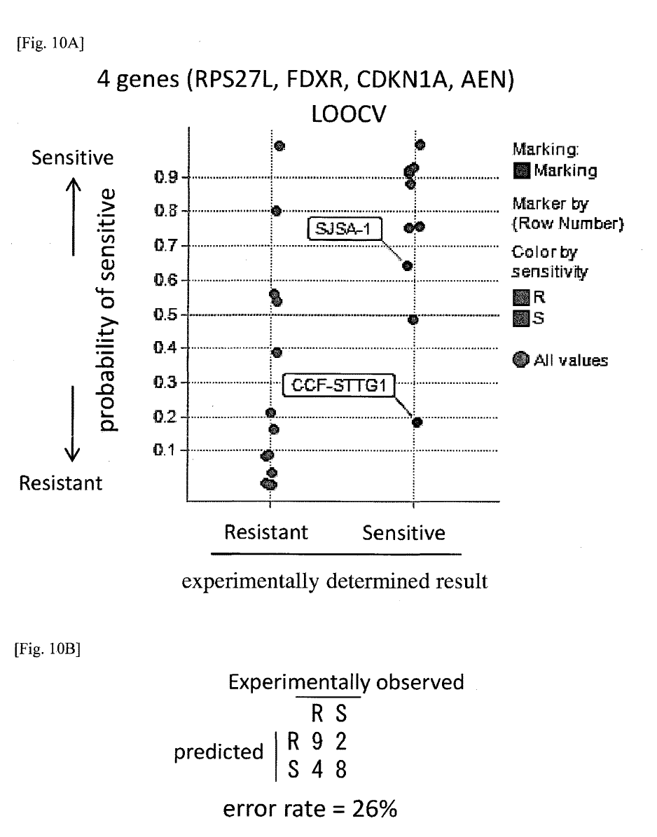

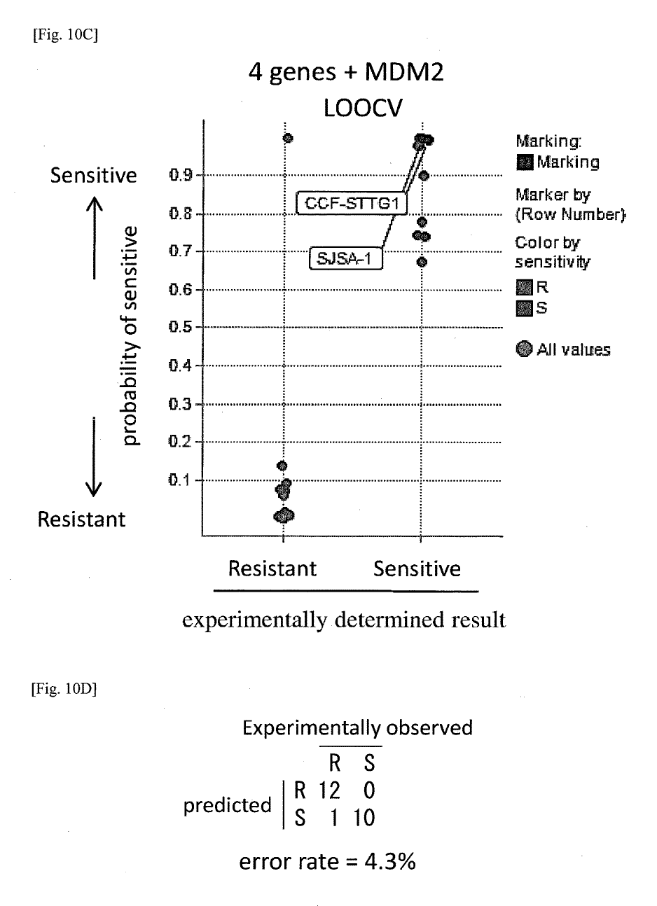

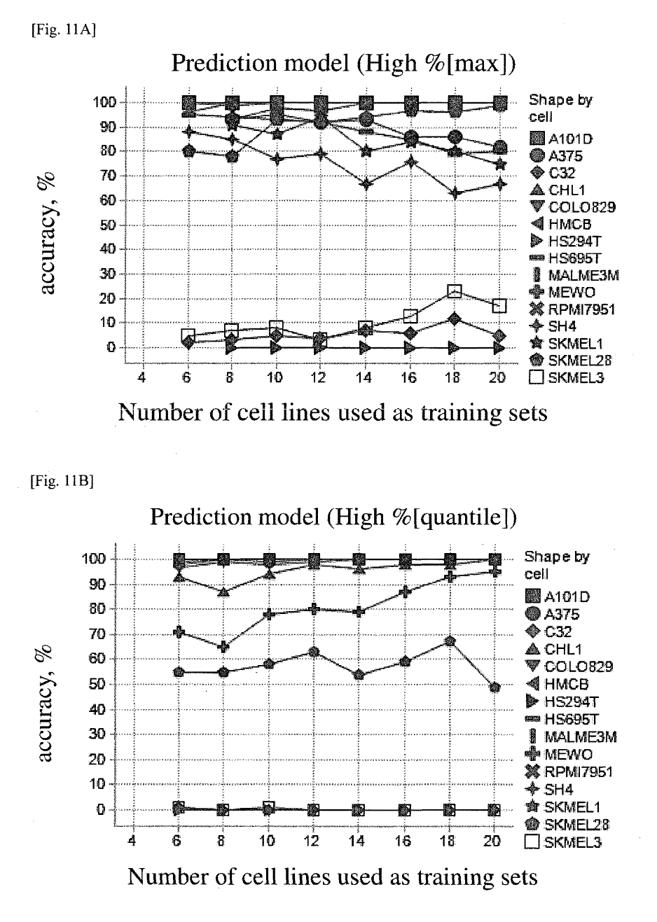

The inventors have also discovered that adding MDM2 to the 4 prediction markers improves the prediction accuracy dramatically (see FIG. 10A-10D). The inventors also found that SJSA-1 and CCF-STTG1 cell lines have MDM2 gene amplification on its genome, and that the expression levels of MDM2 in SJSA-1 and CCF-STTG1 cell lines were far beyond those in the other cell lines (see FIG. 9). It should be noted that the prediction accuracy was improved for all of the other cell lines besides the cell lines SJSA-1 and CCF-STTG1, which have amplified MDM2genes in its genome.

### Example 8

### Effect of a Training Set Size on Prediction Accuracy of Sensitivities to MDM2i Treatment

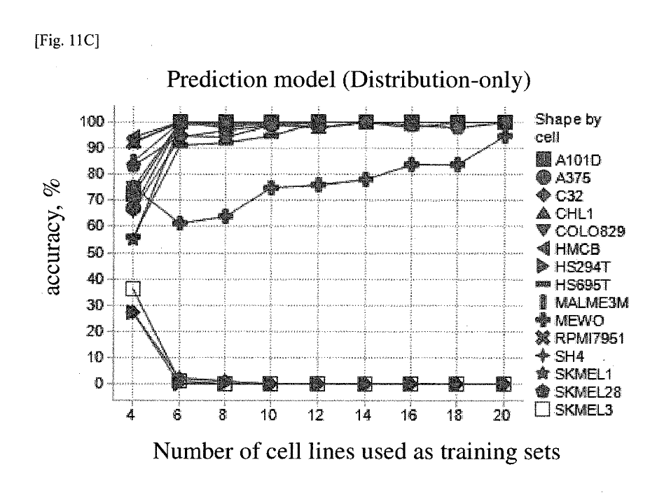

In this example, it was examined whether or not a training set size can impact on prediction accuracy of sensitivities to MDM2i treatment. As shown in FIGS. 11A to 11C, the indicated number of the CCLE cell lines, which included the equal number of TP53 wild type and TP 53 mutant cell lines, were randomly selected and used as a training set to obtain a threshold in the prediction models such as a high %[max] model, a high %[quantile] model and a distribution-only model in each prediction. After obtaining the threshold, the sensitivity of 15 cell lines indicated in the figures were predicted in each model. The sensitivity of each cell was predicted 100 times using 100 different training sets which were randomly selected from cell lines in each prediction. Then, prediction accuracies (%) were calculated from the results.

As shown in FIGS. 11A to 11C, it was demonstrated that the accuracies were kept good in various training set sizes in all the prediction models. In particular, it was demonstrated that 4 cells as a training set size were sufficient to obtain good results in any of the prediction models. No clear dependency on training set size was observed when the training set size was 6 cells or more (FIGS. 11A to 11C).

It is to be understood that suitable methods and materials are described herein for the practice of the embodiments; however, methods and materials that are similar or equivalent to those described herein can be used in the practice or testing of the invention and described embodiments. The nucleic acid sequences corresponding to the publicly available GenBank Accession numbers mentioned herein are incorporated by reference in their entireties.

All publications, patent applications, patents, and other published references mentioned herein are incorporated by reference in their entireties.

1. A method of predicting the sensitivity of a subject's cancer or tumor to MDM2i treatment, comprising measuring the levels of expression of at least three genes selected from the group consisting of RPS27L, FDXR, CDKN1A, AEN, RRM2B, SESN1, TRIAP1, DDB2, CCNG1, XPC, RPL22L1, C12orf5, PPM1D, BAX, BLOC1S2, PHLDA3, PHF23, ZMAT3, FBX022, SPATA18, MDM2, CYFIP2, C1QBP, SPAG7, MAA1967, EDA2R, TNFRSF10B, TP53INP1, SCO1, ZNF828, CLN8, UBFD1, ACTA2, SLC25A11, WDR61, ZSWIM7, NCRNA00188, SPCS1, SMAD4, CCRN4L, PHB2, GRSF1, GAMT, PPID, CNO, OSTC, PSMB6, TMEM131, TYMS, UTP3, LEPROTL1, FOXRED2, EIF4E, RCBTB1, FAM119A, GDF15, MFSD5, BBC3, C19orf60, CNPY2, TM7SF3, ARLSA, FAM98A, TXNL1, ASTN2, ATP5G3, CCDC135, PKD2, SSTR4, LOC731139, ART5, PEBP1, COX18, UBE2G1, ACADS, RAD51C, TARBP2, ARSB, STX8, SLC35B4, NUDT9, MED31, POLH, POLDIP3, A4GNT, RAB3A, SALL1, MAP2K4, ME2, GREB1, FXN, MRPL44, MRPS23, C17orf81, PAPPA2, HDDC2, SLC22A13, FBN3, MFAP3L, C18orf55, ACADSB, TYRO3, TSPAN14, LSMD1, FAM193A, CDK2, DIMT1L, SLC25A30, TTTY11, DCP1B, PDE12, EIF2D, LAMA1, TIMM22, COX10, GLDC, UXS1, CDH20, PDYN, LLGL1, GABARAP, PFAS, PRKY, PRDX3, C17orf71, CNNM2, PCDP1, MED11, USF1, LONP1, TEX19, IFFO1, NAP1L1, FAS, VPS25, TP53, TECTB, KIAA1467, MAPK12, CRADD, LOC643659, NDUFB2, TRAPPC1, HHAT, MRPL19, C18orf32, TSFM, NOP14, MPDU1, GPR84, UBA52, ISCU, IL3, METRN, KCTD7, ZNF425, GDPD1, LOC100129857, PDLIM2, ZNF746, SLC20A1, TNFSF9, EIF1AY, PRTG, TXN2, ORICH2, EIF1B, MLF2, SNX10, MRPL51, ANKRD52, TMEM93, C12orf26, POLR3K, C21orf57, DISC1, and PRPF8 in a cancer or tumor sample obtained from the subject.

2. A method of predicting the sensitivity of a subject's cancer or tumor to MDM2i treatment, comprising:
a) measuring the levels of expression of at least three genes selected from the genes listed in claim 1 in a cancer or tumor sample obtained from the subject; and
b) determining if the cancer or tumor sample has a wild-type TP53 gene.

3. The method according to claim 1, wherein the at least three genes are all of the genes in claim 1.

4. The method according to claim 1, wherein the genes are selected from the group consisting of are BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.

5. The method according to claim 1, wherein the genes are selected from the group consisting of are RPS27L, FDXR, CDKN1A and AEN.

6. The method according to claim 1, wherein measuring the levels of expression of genes comprises measuring the levels of expression of mRNA.

7. The method according to claim 1, wherein measuring the levels of expression of the genes comprises measuring the levels of expression of proteins encoded by the genes.

8. The method according to claim 1, wherein the MDM2i is a spirooxindole derivative, an indole derivative, a pyrrolidine-2-carboxamide derivative, a pyrrolidinone derivative, an isoindolinone derivative, or an imidazothiazole derivative.

9. The method according to claim 1, wherein the MDM2i is Compound A or a salt thereof, Compound B or a salt thereof, CGM097, RG7388, MK-8242 (SCH900242), MI-219, MI-319, MI-773, MI-888, Nutlin-3a, RG7112 (R05045337), TDP521252, TDP665759, PXN727, or PXN822.

10. The method according to claim 1, wherein the MDM2i is Compound A or a salt thereof, or Compound B or a salt thereof.

11. A method of treating an individual having a cancer or tumor, comprising:
c) assessing the sensitivity of a subject's cancer or tumor to MDM2i treatment, comprising measuring the levels of expression of at least three genes selected from the genes listed in claim 1 in a cancer or tumor sample obtained from the subject; and
d) if the assessment indicates that the cancer or tumor is sensitive to the MDM2i, administering to the individual an effective amount of an MDM2i to treat the cancer or tumor.

12. A method of treating an individual having a cancer or tumor, comprising:
e) assessing the sensitivity of a subject's cancer or tumor to MDM2i treatment, comprising measuring the levels of expression of at least three genes selected from the genes listed in claim 1 in a cancer or tumor sample obtained from the subject;
f) determining if the cancer or tumor has a wild-type TP53 gene; and
g) if the assessment a) indicates that the cancer or tumor is sensitive to the MDM2i and the cancer or tumor specimen has a wild-type TP53 gene, administering to the individual an effective amount of an MDM2i to treat the cancer or tumor.

13. The method according to claim 11, wherein the at least three genes are all of the genes in claim 11.

14. The method according to claim 11, wherein the genes are selected from the group consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.

15. The method according to claim 11, wherein the genes are selected from the group consisting of RPS27L, FDXR, CDKN1A and AEN.

16. The method according to claim 11, wherein the levels of expression of genes is the expression of mRNA.

17. The method according to claim 11, wherein the levels of expression of genes is the expression of protein encoded by the genes.

18. The method according to claim 11, wherein the MDM2i is a spirooxindole derivative, an indole derivative, a pyrrolidine-2-carboxamide derivative, a pyrrolidinone derivative, an isoindolinone derivative, or an imidazothiazole derivative.

19. The method according to claim 11, wherein the MDM2i is Compound A or a salt thereof, Compound B or a salt thereof, CGM097, RG7388, MK-8242 (SCH900242), MI-219, MI-319, MI-773, MI-888, Nutlin-3a, RG7112 (R05045337), TDP521252, TDP665759, PXN727, or PXN822.

20. The method according to claim 11, wherein the MDM2i is Compound A or a salt thereof or Compound B or a salt thereof.

21. A gene signature for predicting the sensitivity of a subject's cancer or tumor to MDM2i treatment consisting of at least three genes selected from the genes listed claim 1.

22. The gene signature according to claim 21, wherein the genes are selected from the group consisting of are BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.

23. The gene signature according to claim 21, wherein the genes are selected from the group consisting of are RPS27L, FDXR, CDKN1A and AEN.

24. The gene signature according to claim 21, wherein the MDM2i is a spirooxindole derivative, an indole derivative, a pyrrolidine-2-carboxamide derivative, a pyrrolidinone derivative, an isoindolinone derivative, or an imidazothiazole derivative.

25. The gene signature according to claim 21, wherein the MDM2i is Compound A or a salt thereof, Compound B or a salt thereof, CGM097, RG7388, MK-8242 (SCH900242), MI-219, MI-319, MI-773, MI-888, Nutlin-3a, RG7112 (R05045337), TDP521252, TDP665759, PXN727, or PXN822.

26. A composition comprising a plurality of nucleic acid probes for detecting at least three genes listed claim 1.

27. The composition according to claim 26, wherein the at least three genes are all of the genes in claim 26.

28. The composition according to claim 26, wherein the at least three genes are selected from the group consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.

29. The composition according to claim 26, wherein the at least three genes are selected from the group consisting of RPS27L, FDXR, CDKN1A and AEN.

30. The composition according to claim 26, wherein the plurality of nucleic acid probes comprises an array or a microarray.

31. A kit comprising reagents for the detection of at least three genes listed claim 1, which are indicative of sensitivity to an MDM2i and instructions for use.

32. A kit for predicting sensitivity of a cancer or tumor sample to an MDM2i, said kit comprising nucleic acid probes that specifically bind to nucleotide sequences corresponding to at least three genes listed claim 1, and a means of labeling the nucleic acids.

33. A kit for predicting sensitivity of a cancer or tumor sample to an MDM2i, said kit comprising antibodies or ligands that specifically bind to polypeptides encoded by at least three genes listed claim 1, and a means of labeling the antibodies or ligands that specifically bind to polypeptides or peptides encoded by the genes.

34. The kit according to claim 31, wherein the at least three genes are all of the genes in claim 31.

35. The kit according to claim 31, wherein the at least three genes are selected from the group consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.

36. The kit according to claim 31, wherein the at least three genes are selected from the group consisting of RPS27L, FDXR, CDKN1A and AEN.

37. The kit according to claim 31, wherein the MDM2i is a spirooxindole derivative, an indole derivative, a pyrrolidine-2-carboxamide derivative, a pyrrolidinone derivative, an isoindolinone derivative, or an imidazothiazole derivative.

38. The kit according to claim 31, wherein the MDM2i is Compound A or a salt thereof, Compound B or a salt thereof, CGM097, RG7388, MK-8242 (SCH900242), MI-219, MI-319, MI-773, MI-888, Nutlin-3a, RG7112 (R05045337), TDP521252, TDP665759, PXN727, or PXN822.

39. The kit according to claim 31, wherein the MDM2i is Compound A or a salt thereof or Compound B or a salt thereof.

40. A method for predicting the sensitivity of a subject's cancer or tumor to MDM2i treatment, comprising:
a) measuring the levels of expression of genes comprising at least three genes selected from the genes listed in claim 1 in a cancer or tumor sample obtained from the subject;
b) scoring the levels of expression of the genes obtained in step a) to obtain the subject's sensitivity score;
c) measuring the levels of expression of the genes in plurality of cancer samples or tumor samples, wherein sensitivities to MDM2i treatment of at least a part of the samples are unknown;
d) scoring the levels of expression of the genes obtained in step c) to obtain a reference score in each sample and determining a threshold based on the distribution of the reference scores; and
e) predicting that the subject is sensitive to MDM2i treatment if the subject's sensitivity score is over the threshold and the subject is resistant to MDM2i treatment if the subject's sensitivity score is under the threshold.

41. The method according to claim 40, further comprising:
f) predicting that the subject is sensitive to MDM2i treatment if the subject that is predicted as resistant in step e) shows an MDM2 overexpression.

42. The method according to claim 40, further comprising:
f) predicting that the subject is sensitive to MDM2i treatment if the subject that is predicted as resistant in step e) shows an MDM2 overexpression and has wild type TP53 genes.

43. The method according to claim 41, wherein the MDM2 overexpression is caused by an amplification of MDM2 genes in the genome of the subject.

44. The method according to claim 40, wherein steps b) and d) comprise summing the normalized scores (z-scores) of the levels of the gene expression to obtain the subject's sensitivity score.

45. The method according to claim 44, wherein the threshold in e) ranges between −0.2 and 0.5.

46. The method according to claim 40, wherein the threshold in e) ranges between the values of the third quartile and the maximum of the reference scores of TP53 mutant samples among the samples; or between the values of the first quartile and the minimum of the reference scores of TP53 wild type samples among the samples.

47. The method according to claim 40, wherein the threshold is determined based on Receiver Operating Characteristic (ROC) plots optionally by conducting leave-one-out cross-validation (LOOCV) analysis.

48. The method according to claim 47, wherein the threshold falls within the Youden Index±0.3 of the Receiver Operating Characteristic (ROC) curve.

49. The method according to claim 40, wherein the threshold is determined from the shape of the distribution of the reference scores by using a binalization algorithm.

50. The method according to claim 40, wherein the threshold is determined by Gaussian Mixture model.

51. The method according to claim 50, wherein the threshold is determined based on the ratios of the number of the genes indicating the subject as sensitive to that of the genes indicating the subject as resistant by using two Gaussian distribution in Gaussian Mixture model in step d).

52. The method according to claim 51, wherein the threshold in step e) ranges between the values of the third quartile and the maximum of the ratios of the TP53 mutant samples among the samples; or between the values of the first quartile and the minimum of the ratios of the TP53 wild type samples among the samples.

53. The method according to claim 40, wherein the at least three genes are all of the genes in claim 40.

54. The method according to claim 40, wherein the genes are selected from the group consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.

55. The method according to claim 40, wherein the genes selected from the group consisting of RPS27L, FDXR, CDKN1A and AEN.

56. The method according to claim 40, wherein the genes selected from the group consisting of RPS27L, FDXR, CDKN1A, AEN and MDM2.

57. The method according to claim 40, wherein measuring the levels of expression of genes comprises measuring the levels of expression of mRNA.

58. The method according to claim 40, wherein measuring the levels of expression of the genes comprises measuring the levels of expression of proteins encoded by the genes.

59. The method according to claim 40, wherein the MDM2i is a spirooxindole derivative, an indole derivative, a pyrrolidine-2-carboxamide derivative, a pyrrolidinone derivative, an isoindolinone derivative, or an imidazothiazole derivative.

60. The method according to claim 40, wherein the MDM2i is Compound A or a salt thereof, Compound B or a salt thereof, CGM097, RG7388, MK-8242 (SCH900242), MI-219, MI-319, MI-773, MI-888, Nutlin-3a, RG7112 (R05045337), TDP521252, TDP665759, PXN727, or PXN822.

61. The method according to claim 60, wherein the MDM2i is Compound A or a salt thereof, or Compound B or a salt thereof.

62. A method for predicting the sensitivities of at least a part of subjects' cancers or tumors to MDM2i treatment, comprising:
a′) measuring the levels of expression of genes comprising at least three genes selected from the genes listed in claim 1 in all cancer or tumor samples obtained from all of the subjects whose sensitivities to MDM2i treatment are unknown;
b′) scoring the levels of expression of the genes obtained in step a) to obtain all of the subjects' sensitivity scores and determining a threshold based on the distribution of the sensitivity scores; and
e) predicting that the subjects whose sensitivity scores are over the threshold are sensitive to MDM2i treatment and that the subjects whose sensitivity scores are under the threshold are resistant to MDM2i treatment.

63. The method according to claim 62, further comprising:
f) predicting that subjects are sensitive to MDM2i treatment if the subjects that are predicted as resistant in step e) show an MDM2 overexpression.

64. The method according to claim 62, further comprising:
f) predicting that subjects are sensitive to MDM2i treatment if the subjects that are predicted as resistant in step e) show an MDM2 overexpression and have wild type TP53 genes.

65. The method according to claim 63, wherein the MDM2 overexpression is caused by an amplification of MDM2 genes in the genome of the subject.

66. A method for treating a subject having a cancer or tumor, comprising:
a) assessing the sensitivity of a subject's cancer or tumor to MDM2i treatment by the method according to claim 40; and
b) if the assessment indicates that the cancer or tumor is sensitive to the MDM2i, administering to the subject an effective amount of an MDM2i to treat the cancer or tumor.

67. A pharmaceutical composition for use in treating a cancer or tumor in a subject,
wherein the composition comprises an MDM2i, and
wherein the subject has been predicted as sensitive to the MDM2i treatment by assessing the sensitivity of a subject's cancer or tumor to the MDM2i treatment by the method according to claim 40.
